(19) United States
(12) Patent Application Publication
(10) Pub. No.: US 2023/0365936 A1
(43) Pub. Date: Nov. 16, 2023

(54) GENERATION OF SECRETOME-CONTAINING COMPOSITIONS, AND METHODS OF USING AND ANALYZING THE SAME

(71) Applicants: FUJIFILM Corporation, Tokyo (JP); Assistance Publique - Hôpitaux de Paris, Paris (FR)

(72) Inventors: Nisa K. E. RENAULT, Madison, WI (US); Michele L. Hamrick, Madison, WI (US); Chad H. Koonce, Madison, WI (US); Philippe Menasche, Paris (FR); Valerie Bellamy, Paris (FR); Camille Humbert, Paris (FR); Guillaume Churlaud, Paris (FR); Jerome Larghero, Paris (FR)

(73) Assignees: FUJIFILM Corporation, Tokyo (JP); Assistance Publique - Hôpitaux de Paris, Paris (FR)

(21) Appl. No.: 18/319,224

(22) Filed: May 17, 2023

Related U.S. Application Data
(63) Continuation of application No. PCT/IB2021/000793, filed on Nov. 17, 2021.
(60) Provisional application No. 63/115,230, filed on Nov. 18, 2020.

(51) Int. Cl. C12N 5/0775; C12N 5/00; A61P 9/10

(52) U.S. Cl. CPC C12N 5/0662; C12N 5/0031; A61P 9/10; C12N 2501/998; C12N 2501/115

(57) ABSTRACT

The present disclosure provides methods for generating and/or purifying secretomes, extracellular vesicles, and fractions thereof, from progenitor cells; and provides compositions containing such generated secretomes, extracellular vesicles, and fractions thereof. The present disclosure further provides methods for analyzing activities, and the functionality and potency, of such secretomes, extracellular vesicles, and fractions thereof. The present disclosure also relates to the therapeutic use of secretomes, extracellular vesicles, and fractions thereof. The present disclosure further relates to a good manufacturing practices (GMP)-ready, scalable, culture protocol for the release of clinic-ready secretomes.

### CROSS-REFERENCE TO RELATED APPLICATIONS

This application is a Continuation of PCT International Application No. PCT/IB2021/000793 filed on Nov. 17, 2021, which claims priority under 35 U.S.C. § 119(a) to U.S. Provisional Patent Application No. 63/115,230 filed on Nov. 18, 2020. Each of the above application(s) is hereby expressly incorporated by reference, in its entirety, into the present application.

### FIELD OF THE INVENTION

The present disclosure relates generally to the generation, purification, isolation, and/or enrichment, of secretomes from cells (such as, but not limited to, progenitor cells); secretome-containing compositions containing such generated, purified, isolated, and/or enriched, secretomes; and to methods for analyzing one or more activities, properties, and/or characteristics, of such secretome-containing compositions. The present disclosure also relates to the therapeutic use of secretome-containing compositions containing secreted bioactive molecules, produced, purified, isolated, and/or enriched, by a method or methods disclosed herein. The present disclosure further relates to good manufacturing practices (GMP)-ready, scalable, culture protocols for the release, purification, isolation, and/or enrichment, of clinic-ready secretomes.

### BACKGROUND INFORMATION

Cells, including those in in vitro or ex vivo culture, secrete a large variety of molecules and biological factors (collectively known as a secretome) into the extracellular space. See Vlassov et al. (Biochim Biophys Acta, 2012; 940-948). As part of the secretome, various bioactive molecules are secreted from cells within membrane-bound extracellular vesicles, such as exosomes. Extracellular vesicles are capable of altering the biology of other cells through signaling, or by the delivery of their cargo (including, for example, proteins, lipids, and nucleic acids). The cargo of extracellular vesicles is encased in a membrane which, amongst others, allows for specific targeting (e.g., to target cells) via specific markers on the membrane; and increased stability during transport in biological fluids, such as through the bloodstream or across the blood-brain-barrier (BBB).

Exosomes exert a broad array of important physiological functions, e.g., by acting as molecular messengers that traffic information between different cell types. For example, exosomes deliver proteins, lipids and soluble factors including RNA and microRNAs which, depending on their source, participate in signaling pathways that can influence apoptosis, metastasis, angiogenesis, tumor progression, thrombosis, immunity by directing T cells towards immune activation, immune suppression, growth, division, survival, differentiation, stress responses, apoptosis, and the like. See Vlassov et al. (Biochim Biophys Acta, 2012; 940-948). Extracellular vesicles may contain a combination of molecules that may act in concert to exert particular biological effects. Exosomes incorporate a wide range of cytosolic and membrane components that reflect the properties of the parent cell. Therefore, the terminology applied to the originating cell can in some instances be used as a simple reference for the secreted exosomes.

Progenitor cells have proliferative capacity and can differentiate into mature cells, making progenitor cells attractive for therapeutic applications such as regenerative medicine, e.g., in treating myocardial infarction and congestive heart failure. It has been reported that extracellular vesicles secreted by stem cell-derived cardiovascular progenitor cells produce similar therapeutic effects to their secreting cells in a mouse model of chronic heart failure, see Kervadec et al. (J. Heart Lung Transplant, 2016; 35:795-807), suggesting that a significant mechanism of action of transplanted progenitor cells is in the release of biological factors following transplantation (e.g., which stimulate endogenous regeneration or repair pathways). This raises the possibility of effective, cell-free therapies (with benefits such as improved convenience, stability, and operator handling). See El Harane et al. (Eur. Heart J., 2018; 39:1835-1847). However, there currently is a need for improved production methods for generating, purifying, isolating, and/or enriching, extracellular vesicles.

For instance, regulatory approval of production of drugs and biological substances requires strict adherence to laws and regulations that are promulgated with the goal of establishing safe and effective manufacturing facilities and products. As a non-limiting example, “Good Manufacturing Practices” (GMP) and “Good Laboratory Practices” (GLP) are established by regulation and implemented by the FDA (the U.S. Food and Drug Administration), CDER (Center for Drug Evaluation and Research), and CBER (Center for Biologics Evaluation and Research), with regard to drugs and biologics. Similar GMP and/or GLP laws are implemented worldwide, for instance in the EMEA.

However, established techniques for the generation of extracellular vesicles typically employ reagents and/or conditions that are not compatible with clinical or therapeutic use, or GMP standards. For example, the use of serum in culturing protocols raises reliability- and biosafety-concerns, especially where serum obtained from an animal may be contaminated with, for example, infectious agents such as viruses or prions. Fetal bovine serum (FBS) is a widely used growth supplement for cell and tissue culture media; however, FBS is not well suited for clinical or therapeutic use for these reasons.

In contrast, the use of serum-free media confers many advantages, including consistency in formulations and safety. However, using only serum-free media can have disadvantageous effects on cell metabolism and growth, and there exists a need for good manufacturing practices (GMP)-ready compositions/methods for generating, purifying, isolating, and/or enriching, secretome compositions.

### SUMMARY OF THE INVENTION

The present disclosure addresses the above-described limitations in the art, by providing methods for generating, purifying, isolating, and/or enriching, secretomes using serum-free media, thereby permitting a GMP-ready, scalable, quality-controlled culture protocol for the release of clinic-ready secretomes.

The present disclosure also provides methods for generating, purifying, isolating, and/or enriching, secretomes, extracellular vesicles, and fractions thereof, from cells (such as, but not limited to, progenitor cells); and provides compositions containing such generated, purified, isolated, and/or enriched, secretomes, extracellular vesicles, and fractions thereof. The present disclosure further provides methods for analyzing one or more activities, properties, and/or characteristics, of such secretomes, extracellular vesicles, and fractions thereof, as well as the therapeutic use of secretomes, extracellular vesicles, and fractions thereof.

Non-limiting embodiments of the disclosure include as follows:
[1] A method for generating a secretome, said method comprising: (a) culturing one or more progenitor cells in a first serum-free culture medium, wherein said first serum-free culture medium comprises basal medium, human serum albumin, and one or more growth factors; (b) removing said first serum-free culture medium from said one or more progenitor cells; (c) culturing said one or more progenitor cells in a second serum-free culture medium, wherein said second serum-free culture medium comprises basal medium, but does not comprise human serum albumin or growth factors; and (d) recovering the second serum-free culture medium after the culturing of step (c), to thereby obtain conditioned medium comprising the secretome of the one or more progenitor cells.
[2] The method of [1], wherein one of said one or more growth factors is fibroblast growth factor 2 (FGF-2).
[3] The method of [1] or [2], wherein said first and second serum-free media are supplemented with a carbohydrate source.
[4] The method of [3], wherein said carbohydrate source is glucose.
[5] The method of any one of [1]-[4], wherein said first and second serum-free media are supplemented with an antibiotic.
[6] The method of [5], wherein said antibiotic is gentamicin.
[7] The method of any one of [1]-[6], wherein said first serum-free media further comprises one or more selected from the group consisting of: glutamine; biotin; DL alpha tocopherol acetate; DL alpha-tocopherol; vitamin A; catalase; insulin; transferrin; superoxide dismutase; corticosterone; D-galactose; ethanolamine, glutathione; L-carnitine; linoleic acid; progesterone; putrescine; sodium selenite; triodo-I-thyronine; an amino acid; sodium pyruvate; lipoic acid; vitamin B12; nucleosides; and ascorbic acid.
[8] The method of any one of [1]-[7], wherein said basal medium is a Minimum Essential Medium (MEM).
[9] The method of [8], wherein said MEM is α-MEM.
[10] The method of any one of [1]-[9], wherein the culturing of step (a) is for 6-96 hours.
[11] The method of [10], wherein the culturing of step (a) is for 12-96 hours.
[12] The method of [11], wherein the culturing of step (a) is for 36-84 hours.
[13] The method of [12], wherein the culturing of step (a) is for about 72 hours.
[14] The method of any one of [1]-[13], wherein the culturing of step (c) is for 6-96 hours.
[15] The method of [14], wherein the culturing of step (c) is for 12-72 hours.
[16] The method of [15], wherein the culturing of step (c) is for 36-60 hours.
[17] The method of [16], wherein the culturing of step (c) is for about 48 hours.
[18] The method of [14], wherein the last 12-36 hours of the culturing of step
(c) is conducted under hypoxic conditions.
[19] The method of [18], wherein said culture conditions comprises culturing in an atmosphere having 1-21% oxygen.
[20] The method of any one of [1]-[19], wherein after step (b), but before step (c), said one or more progenitor cells are washed.
[21] The method of any one of [1]-[20], wherein said one or more progenitor cells comprise progenitor cells selected from the group consisting of cardiomyocyte progenitor cells, cardiac progenitor cells, and cardiovascular progenitor cells.
[22] The method of any one of [1]-[21], wherein said one or more progenitor cells are obtained from induced pluripotent stem cells (iPSCs).
[23] The method of any one of [1]-[4] and [7]-[22], wherein said first and second serum-free media do not contain an antibiotic.
[24] The method of any one of [1]-[23], wherein the culturing in one or more of steps (a) and (c) is two-dimensional cell culture.
[25] The method of [24], wherein said two-dimensional cell culture comprises culturing said one or more progenitor cells on a surface of a culture vessel.
[26] The method of [25], wherein said culture vessel surface is coated with a substance to promote cell adhesion.
[27] The method of [26], wherein said substance to promote cell adhesion is vitronectin or fibronectin.
[28] The method of any one of [1]-[23], wherein the culturing in one or more of steps (a) and (c) is three-dimensional cell culture.
[29] The method of [28], wherein the three-dimensional cell culture comprises culturing cell aggregates in suspension in a bioreactor, spinner flask, or stirred culture vessel, or comprises culturing cells in a microcarrier culture system.
[30] The method of any one of [1]-[29], wherein said method further comprises pre-clearing the medium recovered in step (d) by centrifugation, filtration, or a combination of centrifugation and filtration.
[31] The method of any one of [1]-[30], wherein said method further comprises freezing the medium recovered in step (d).
[32] The method of any one of [1]-[31], wherein said one or more progenitor cells cultured in step (a) have previously been frozen.
[33] The method of any one of [1]-[32], wherein said method further comprises concentrating, and/or enriching for, a small extracellular vesicle-enriched fraction (sEV) from the medium recovered in step (d).
[34] The method of [33], wherein said sEV is concentrated, and/or enriched, from the recovered medium by at least one process selected from the group consisting of ultracentrifugation, filtration, ultrafiltration, tangential flow filtration, size exclusion chromatography, and affinity capture.
[35] The method of [33], wherein said enriching enriches for extracellular vesicles that have one or more of the following characteristics: (a) are CD63⁺, CD81⁺ and/or CD9⁺; (b) are between 50-200 nm in diameter; (c) are positive for one or more of CD49e, ROR1 (Receptor Tyrosine Kinase Like Orphan Receptor 1), SSEA-4 (Stage-specific embryonic antigen 4), MSCP (Mesenchymal stem cell-like protein), CD146, CD41b, CD24, CD44, CD236, CD133/1, CD29 and CD142; and/or (d) are negative for one or more of CD19, CD4, CD209, HLA-ABC (human leukocyte antigen-ABC), CD62P, CD42a and CD69.
[36] The method of [33], wherein said sEV comprises one or more of exosomes, microparticles, extracellular vesicles and secreted peptides/proteins.
[37] A secretome-containing composition obtained by the method of any one of [1]-[32].
[38] An sEV-containing composition obtained by the method of any one of [33]-[36].
[39] A method for producing a therapeutic composition suitable for administration to a patient, said method comprising producing a secretome-containing composition according to the method of any one of [1]-[32].
[40] The method of [39], wherein said method further comprises purifying, concentrating, isolating, and/or enriching, said secretome-containing composition by one or more purification, concentrating, isolation, and/or enrichment, steps.
[41] The method of [39], wherein said method further comprises adding a pharmaceutically acceptable excipient or carrier to the secretome-containing composition.
[42] A method for producing a therapeutic composition suitable for administration to a patient, said method comprising producing an sEV-containing composition according to the method of any one of [33]-[36].
[43] The method of [42], wherein said method further comprises purifying, concentrating, isolating, and/or enriching, said sEV-containing composition by one or more purification, concentration, isolation, and/or enrichment, steps.
[44] The method of [42], wherein said method further comprises adding a pharmaceutically acceptable excipient or carrier to the sEV-containing composition.
[45] A therapeutic composition, wherein said therapeutic composition comprises the secretome-containing composition of [37], and a pharmaceutically acceptable excipient or carrier.
[46] A therapeutic composition, wherein said therapeutic composition comprises the sEV-containing composition of [38], and a pharmaceutically acceptable excipient or carrier.
[47] A secretome-containing composition obtained by the method of [1], wherein said one or more progenitor cells comprise progenitor cells selected from the group consisting of cardiomyocyte progenitor cells, cardiac progenitor cells, and cardiovascular progenitor cells.
[48] An sEV-containing composition obtained by the method of [33], wherein said one or more progenitor cells comprise progenitor cells selected from the group consisting of cardiomyocyte progenitor cells, cardiac progenitor cells, and cardiovascular progenitor cells.
[49] A therapeutic composition, wherein said therapeutic composition comprises the composition of [47], and a pharmaceutically acceptable excipient or carrier.
[50] A therapeutic composition, wherein said therapeutic composition comprises the composition of [48], and a pharmaceutically acceptable excipient or carrier.
[51] A method for treating acute myocardial infarction or heart failure, comprising administering to a subject in need thereof the therapeutic composition of [49] or [50].
[52] A method for improving angiogenesis, comprising administering to a subject in need thereof the therapeutic composition of [49] or [50].
[53] A method for improving cardiac performance, comprising administering to a subject in need thereof the therapeutic composition of [49] or [50].
[54] The method of [11], wherein the culturing of step (a) is for 60-84 hours.
[55] The method of [14], wherein the last 12-36 hours of the culturing of step (c) is conducted under normoxic conditions.
[56] The method of [55], wherein said normoxic conditions comprises culturing in an atmosphere containing 20-21% oxygen.
[57] The method of [29], wherein the bioreactor is a vertical wheel bioreactor.
[58] The method of [39], wherein said method further comprises cryopreserving, freezing, or lyophilizing, said secretome-containing composition.
[59] The method of [42], wherein said method further comprises cryopreserving, freezing, or lyophilizing, said sEV-containing composition.
[60] The method of [2], wherein said first serum-free media comprises 0.1-10 μg/mL FGF-2.
[61] The method of [60], wherein said first serum-free media comprises 0.5-5 μg/mL FGF-2.
[62] The method of [61], wherein said first serum-free media comprises 0.5-2.5 μg/mL FGF-2.
[63] The method of [62], wherein said first serum-free media comprises about 1 μg/mL FGF-2.
[64] The method of any of [1]-[36], [39]-[44] and [54]-[63], wherein said method is Good Manufacturing Practices (GMP)-ready.
[65] The secretome-containing composition of [37], wherein said composition is GMP-ready.
[66] The sEV-containing composition of [38], wherein said composition is GMP-ready.
[67] The method of [14], wherein the last 12-36 hours of the culturing of step (c) is conducted under normoxic conditions.
[68] The method of [67], wherein said normoxic conditions comprises culturing in an atmosphere containing between 20-21% of oxygen.
[69] The method of [30], wherein said pre-clearing comprises at least three filtration steps.
[70] The method of [34], wherein the separation of said sEV from the recovered medium comprises tangential flow filtration.
[71] The secretome-containing composition of [37], wherein said composition comprises trehalose, and optionally, L-histidine.
[72] The sEV-containing composition of [38], wherein said composition comprises trehalose, and optionally, L-histidine.
[73] The secretome-containing composition of [37] or [65], wherein said composition is able to promote wound scratch healing in an in vitro wound scratch healing assay, and/or is able to promote cardiomyocyte viability in an in vitro cardiomyocyte viability assay.
[74] The sEV-containing composition of [38] or [66], wherein said composition is able to promote wound scratch healing in an in vitro wound scratch healing assay, and/or is able to promote cardiomyocyte viability in an in vitro cardiomyocyte viability assay.
[75] The secretome-containing composition of [37] or [65], wherein said composition is at least one of the following: a composition that has been enriched for extracellular vesicles having a diameter of between about 50-200 nm or between 50-200 nm, preferably having a diameter of between about 50-150 nm or between 50-150 nm; a composition that is substantially free or free of whole cells; and/or a composition that is substantially free of one or more culture medium components.
[76] The sEV-containing composition of [38] or [66], wherein said composition is at least one of the following: a composition that has been enriched for extracellular vesicles having a diameter of between about 50-200 nm or between 50-200 nm, preferably having a diameter of between about 50-150 nm or between 50-150 nm; a composition that is substantially free or free of whole cells; and/or a composition that is substantially free of one or more culture medium components.
[77] The method of [51], wherein the heart failure is acute heart failure, chronic heart failure, ischemic heart failure, non-ischemic heart failure, heart failure with ventricular dilation, heart failure without ventricular dilation, heart failure with reduced left ventricular ejection fraction, or heart failure with preserved left ventricular ejection fraction.
[78] The method of [77], wherein the heart failure is selected from the group consisting of ischemic heart disease, cardiomyopathy, myocarditis, hypertrophic cardiomyopathy, diastolic hypertrophic cardiomyopathy, dilated cardiomyopathy, and post-chemotherapy induced heart failure.

### INCORPORATION BY REFERENCE

All patents, publications, and patent applications cited in the present specification are herein incorporated by reference as if each individual patent, publication, or patent application was specifically and individually indicated to be incorporated by reference in its entirety for all purposes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts an iPSC to CPC process flow diagram, illustrating the generation of cardiovascular progenitor cells from hiPSCs (steps 1-4). After CPC generation, cells were maintained as fresh aggregates (5a) or dissociated to single cells (step 5b) for the vesiculation process. Single cells were plated fresh or cryo-preserved and plated post-thaw (steps 6-7) for the vesiculation process.

FIG. 2 depicts flowcharts showing the material generated in Example 1. As shown in FIG. 2, two batches of CPCs (CPC1, CPC2) were generated and each were divided into three vesiculation conditions: aggregate vesiculation, fresh CPC plated vesiculation, and thawed CPC plated vesiculation. The conditioned media from each condition were collected, pre-cleared, and frozen (MC1-6). The cells at the end of four days of the vesiculation process (day+4) were also collected and analyzed (C+4 #1-6). Conditioned media were subjected to ultracentrifugation (UC) to isolate the small vesicular fraction (sEV 1-6). For MC5, three separate rounds of UC were performed on separate aliquots of MC5. In parallel, vessels containing media but no cells were “cultured,” and virgin media were collected (virgin media 1-3), and MV controls were generated via the same UC protocol (MV1.1-3).

FIG. 3 depicts a heat map of the relative gene expression of 48 relevant genes to CPC differentiation and potential off targets. Data were generated using a custom Fluidigm qPCR panel. Data from CPCs at the end of the differentiation process (CPC), as well as four days into the vesiculation process (C+4), are shown in addition to iPSC and cardiomyocyte (CM) controls. Under these conditions, CPC are clustered and separate from C+4 cells, which are more mature than CPCs but less mature than CM. Fourth vesiculation day aggregates (Agg+4) are distinct from fourth day hyperflask plated cells (HF+4). Both conditions show increased cTNT (cardiac Troponin T) and alpha-MHC (alpha-myosin heavy chain) expression compared to CPC. This supports the idea that CPC in the vesiculation process remain on the cardiac differentiation lineage, but do not attain the CM differentiation state, as shown by the persistence of CPC marker expression such as PDGFRa, ISL-1 and KDR.

FIG. 4 depicts a process flow diagram for the generation of conditioned media and virgin media controls.

FIG. 5 depicts a process flow diagram for the isolation of sEV or mock (virgin media) control samples.

FIG. 6 depicts representative size distribution curves from two sEVs and two control MV samples. Suspension culture yielded higher concentrations of particles than plated culture, and both were much higher than controls. Mode particle sizes for sEVs (74 nm, 99 nm) are consistent with exosomes or small microparticles.

FIG. 7 depicts ELISA results for the detection of CD-63. sEVs and MV controls were analyzed by FUJIFILM Wako Elisa kit for the detection of CD-63, a protein found on the surface of EV, especially exosomes. The results show that for a given protein input, MVs contain no CD-63 signal, whereas sEVs from aggregate and plated cultures do. Aggregate sEV produced more CD-63/protein signal than sEV from plated vesiculation protocols. Replicate preparations of sEV from the same MC (5.1, 5.2 and 5.3) yielded similar CD63 signals. Furthermore, sEV isolated from different MCs generated from separate lots also yielded similar CD-63/μg protein (sEV 2 vs sEV 5.1/.2/.3).

FIG. 8 depicts relative scratch wound closure in a HUVEC scratch wound healing assay. sEVs from suspension and plated vesiculation processes as well as their corresponding mock EV controls (MV) were tested in a HUVEC scratch wound healing assay. Controls were complete HUVEC media (positive), poor HUVEC media (no supplements, Negative), and poor media+the sEV isolated from fetal bovine serum by UC (FBS-EV, positive control). sEV from suspension and plated vesiculation processes showed improved wound healing compared to Negative and MV controls.

FIG. 9 depicts the results of a H9c2 viability assay. The results of the H9c2 cell viability assay show that the sEVs from suspension and plated cultures improve H9c2 survival in a serum deprivation assay. MVs showed minimal to no positive effect in this assay. sEV generated from the suspension vesiculation method showed an improvement in cell number over the positive control, suggesting increased cell proliferation in addition to sustained survival.

FIG. 10 depicts a time course of cardiomyocyte survival in a staurosporine-induced cardiotoxicity assay. sEV from plated and aggregate cultures improve CM survival in this staurosporine assay. MVs showed little to no effect on CM survival. Arrows link each sEV with its corresponding MV control.

FIGS. 11A and 11B depict flowcharts illustrating the stages of production (vesiculation, conditioned media clarification, and TFF, FIG. 11A; followed by final formulation, FIG. 11B) in a first GMP-compatible process, described in Example 5. The final formulation in this example was produced with and without trehalose addition prior to sterilizing filtration. The different stages at which quality control testing was undertaken are indicated with a “*” (e.g., *1, *2, *3, etc.).

FIG. 12 depicts the results of flow cytometry experiments to analyze the cell marker expression profile of CPCs at different times during the vesiculation process (D+0, D+3 and D+5). iPSCs and cardiomyocytes (CM) were used as control cells, and were analyzed separately. The values shown are average values.

FIG. 13 depicts the results of transcriptome analysis of CPCs at different times during the vesiculation process (D+0, D+3 and D+5). RNA was extracted from CPCs at D+0, and from cells at D+3 and D+5 of the vesiculation process. RNA was also extracted from iPSCs (pluripotent cell controls), and from iPSC-derived cardiomyocytes (differentiated cardiomyocyte controls; CM). Total RNA was sequenced on the Illumina NovaSeq 6000 platform, and differential gene expression was determined on normalized data. The heat map was generated based on hierarchical clustering analysis using the UPGMA clustering method, with correlation distance metric in TIBCO Spotfire software v11.2.0.

FIG. 14 depicts the morphology of CPCs during the vesiculation process, as observed under light microscopy. Cell morphology was analyzed in cells within both 175 and selected CS10 flasks. The left image is a representative image showing the typical D+3 morphology observed in all vessels analyzed at D+3. The right image is a representative image showing the typical D+5 morphology observed in all vessels analyzed at D+5. T75 flasks were used for image capture for clarity.

FIGS. 15A and 15B depict the results of an analysis of particle concentration and size distribution of EVs. FIG. 15A depicts the particle concentration and size distribution of EVs in clarified conditioned media before tangential flow filtration (TFF) (*5), and in final formulations with and without trehalose (*7), using nanoparticle tracking analysis. FIG. 15B depicts the particle concentration and size distribution of EVs in clarified conditioned media before tangential flow filtration (TFF) (*5), and in stored retentate samples (with and without trehalose or histidine) which were not filter sterilized (“*6,” samples a-c). As FIGS. 15A and 15B show, TFF increased the particle concentration by about 32-fold.

FIGS. 16A-16D depict the results of MACSPlex analysis. FIGS. 16A and 16B depict the results of analysis of small EV-enriched secretome final formulations with and without trehalose, for expression of extracellular vesicle tetraspanins often expressed on the surface of extracellular vesicles (CD9, CD81 and CD63) (FIG. 16A); and for various additional markers, which exhibited little or no expression (FIG. 16B). FIGS. 16C and 16D depict the results of analysis of stored retentate samples (with and without trehalose or histidine) which were not filter sterilized (see FIG. 11B, *6, samples a-c), for expression of extracellular vesicle tetraspanins often expressed on the surface of extracellular vesicles (CD9, CD81 and CD63) (FIG. 16C); and for various additional markers, which exhibited little or no expression (FIG. 16D).

FIGS. 17A and 17B depict the results of analysis of EVs for the presence of cardiac-related markers. FIG. 17A depicts the results for small EV-enriched secretome final formulations with and without trehalose, for expression of cardiac-related markers. FIG. 17B depicts the results for stored retentate samples (with and without trehalose or histidine) which were not filter sterilized, for expression of cardiac-related markers.

FIG. 18 depicts relative scratch wound healing in a HUVEC scratch wound healing assay. Small EV-enriched secretome final formulations with and without trehalose, were tested in a HUVEC scratch wound healing assay. The positive control (+ve) consisted of culturing the scratched well in complete HUVEC cell medium (Comp) plus PBS “treatment,” and the negative control (−ve) consisted of culturing the scratched wells in basal medium (Poor) plus PBS “treatment.” FBS-derived EV served as an EV control (EV Ctl). 1×equals the secretome derived from 150,000 cells. Values are baseline subtracted (negative control) and normalized to the positive control.

FIG. 19 depicts cardiomyocyte survival in a staurosporine-induced cardiotoxicity assay. Small EV-enriched secretome final formulations with and without trehalose, were tested in a cardiomyocyte survival assay. 1×equals the secretome derived from 150,000 cells. PBS controls with and without staurosporine served as negative (−ve) and positive (+ve) controls, respectively. Mesenchymal Stem Cell (MSC)-derived EV served as an EV control (EV Ctl). Plated cells were either stressed with staurosporine for 4 hours prior to treatment (+), or were not stressed with staurosporine (−).

FIG. 20 depicts an exemplary secretome/extracellular vesicle process/product testing panel.

FIG. 21 depicts the secretome/extracellular vesicle process/product testing panel relating to Examples 5-17.

FIG. 22 depicts the results for certain criteria shown in the testing panel in FIG. 21, with respect to Examples 5-11.

FIG. 23 depicts the degree of enrichment (as calculated by the increase of particles per unit protein), as compared to conditioned media after clarification, for the retentates and final formulations produced in Example 6.

FIGS. 24A and 24B depict flowcharts illustrating the stages of production (vesiculation, conditioned media clarification, and TFF, FIG. 24A; and final formulation, FIG. 24B) in a second GMP-compatible process, described in Example 12. The final formulation in this example was produced with and without trehalose addition prior to sterilizing filtration. The different stages at which quality control testing was undertaken are indicated with a “*” (e.g., *6, *7, etc.).

FIG. 25 depicts the results of flow cytometry experiments to analyze the cell marker expression profile of CPCs at different times during the vesiculation process (D+0, D+3 and D+5). iPSCs and cardiomyocytes (CM) were used as control cells, and were analyzed separately. The values shown are average values.

FIG. 26 depicts the morphology of CPCs during the vesiculation process, as observed under light microscopy. Cell morphology was analyzed in cells within both T75 and selected CS10 flasks. The left image is a representative image showing the typical D+3 morphology observed in all vessels analyzed at D+3 The right image is a representative image showing the typical D+5 morphology observed in all vessels analyzed at D+5. T75 flasks were used for image capture for clarity.

FIGS. 27A and 27B depict the results of an analysis of particle concentration and size distribution of EVs. FIG. 27A depicts the particle concentration and size distribution of EVs in conditioned media (before and after clarification) before tangential flow filtration (TFF) (*4 and *5), and in final formulations with and without trehalose (*7), using nanoparticle tracking analysis. FIG. 27B depicts the particle concentration and size distribution of EVs in retentate (*6) and previously frozen, filter-sterilized final formulations without trehalose (*7).

FIGS. 28A-28B depict the results of analysis of small EV-enriched secretome final formulations with and without trehalose, for expression of extracellular vesicle tetraspanins often expressed on the surface of extracellular vesicles (CD9, CD81 and CD63) (FIG. 28A); and for various other markers, which exhibited little or no expression (FIG. 28B).

FIG. 29 depicts the results for small EV-enriched secretome final formulations with and without trehalose, for expression of cardiac-related markers.

FIGS. 30A and 30B depict relative scratch wound healing in a HUVEC scratch wound healing assay. The results for samples a and b (depicted in FIG. 24B) are shown in FIG. 30A. The results for samples c and d (depicted in FIG. 24B) are shown in FIG. 30B. The positive control (+ve) consisted of culturing the scratched well in complete HUVEC cell medium (Comp) plus PBS “treatment”, and the negative control (−ve) consisted of culturing the scratched wells in basal medium (Poor) plus PBS “treatment”. FBS-derived EV served as an EV control (EV Ctl). 1×equals the secretome derived from 150,000 cells. Values are baseline subtracted (negative control) and normalized to the positive control.

FIGS. 31A and 31B depict cardiomyocyte survival in a staurosporine-induced cardiotoxicity assay. The results for samples a and b (depicted in FIG. 24B) are shown in FIG. 31A. The results for samples c and d (depicted in FIG. 24B) are shown in FIG. 31B. 1×equals the secretome derived from 150,000 cells. PBS controls with and without staurosporine served as negative (−ve) and positive (+ve) controls, respectively. Mesenchymal Stem Cell (MSC)-derived EV served as an EV control (EV Ctl). Plated cells were either stressed with staurosporine for 4 hours prior to treatment (+), or were not stressed with staurosporine (−).

FIG. 32 depicts the results for certain criteria shown in the testing panel in FIG. 21, with respect to Examples 12-17.

FIG. 33 depicts the degree of enrichment (as calculated by the increase in particles per unit protein), as compared to conditioned media after clarification, for the retentates and final formulations produced in Example 12.

FIG. 34 depicts echocardiography results of mice with induced chronic heart failure following administration of CPC EVs (“sEV5.3”), or PBS (as a control). The data depicts the absolute changes in Left Ventricular End Systolic Volume (LVESV); Left Ventricular End Diastolic Volume (LVEDV); and ejection fraction (EF).

### DETAILED DESCRIPTION OF THE INVENTION

It is to be understood that the terminology used herein is for the purpose of describing particular embodiments only and is not intended to be limiting. As used in the present specification and the appended claims, the singular forms “a,” “an,” and “the” include plural referents unless the context clearly dictates otherwise. Thus, for example, reference to “a cell” includes one or more cells.

Unless defined otherwise, all technical and scientific terms used herein have the same meaning as commonly understood by one of ordinary skill in the art to which the invention pertains. Although other methods and materials similar, or equivalent, to those described herein can be useful in the present invention, preferred materials and methods are described herein.

As used herein, “subject,” “individual,” or “patient” are used interchangeably herein and refer to any member of the phylum Chordata, including, without limitation, humans and other primates, including non-human primates, such as rhesus macaques, chimpanzees, and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys, and other gallinaceous birds, ducks, and geese; and the like. The term does not denote a particular age or gender. Thus, the term includes adult, young, and newborn individuals as well as males and females. In some embodiments, cells (for example, stem cells, including pluripotent stem cells, progenitor cells, or tissue-specific cells) are derived from a subject. In some embodiments, the subject is a non-human subject.

As used herein, “differentiation” refers to processes by which unspecialized cells (such as pluripotent stem cells, or other stem cells), or multipotent or oligopotent cells, for example, acquire specialized structural and/or functional features characteristic of more mature, or fully mature, cells. “Transdifferentiation” is a process of transforming one differentiated cell type into another differentiated cell type.

As used herein, “embryoid bodies” refers to three-dimensional aggregates of pluripotent stem cells. These cells can undergo differentiation into cells of the three germ layers, the endoderm, mesoderm and ectoderm. The three-dimensional structure, including the establishment of complex cell adhesions and paracrine signaling within the embryoid body microenvironment, enables differentiation and morphogenesis.

As used herein, “stem cell” refers to a cell that has the capacity for self-renewal, i.e., the ability to go through numerous cycles of cell division while maintaining their non-terminally-differentiated state. Stem cells can be totipotent, pluripotent, multipotent, oligopotent, or unipotent. Stem cells may be, for example, embryonic, fetal, amniotic, adult, or induced pluripotent stem cells.

As used herein, “pluripotent stem cell” (PSC) refers to a cell that has the ability to reproduce itself indefinitely, and to differentiate into any other cell type of an adult organism. Generally, pluripotent stem cells are stem cells that are capable of inducing teratomas when transplanted in immunodeficient (SCID) mice; are capable of differentiating into cell types of all three germ layers (e.g., can differentiate into ectodermal, mesodermal, and endodermal, cell types); and express one or more markers characteristic of PSCs. Examples of such markers expressed by PSCs, such as embryonic stem cells (ESCs) and iPSCs, include Oct 4, alkaline phosphatase, SSEA-3 surface antigen, SSEA-4 surface antigen, nanog, TRA-1-60, TRA-1-81, SOX2, and REX1.

As used herein, “induced pluripotent stem cell” (iPSC) refers to a type of pluripotent stem cell that is artificially derived from a non-pluripotent cell, typically a somatic cell. In some embodiments, the somatic cell is a human somatic cell. Examples of somatic cells include, but are not limited to, dermal fibroblasts, bone marrow-derived mesenchymal cells, cardiac muscle cells, keratinocytes, liver cells, stomach cells, neural stem cells, lung cells, kidney cells, spleen cells, and pancreatic cells. Additional examples of somatic cells include cells of the immune system, including, but not limited to, B-cells, dendritic cells, granulocytes, innate lymphoid cells, megakaryocytes, monocytes/macrophages, myeloid-derived suppressor cells, natural killer (NK) cells, T cells, thymocytes, and hematopoietic stem cells.

iPSCs may be generated by reprogramming a somatic cell, by expressing or inducing expression of one or a combination of factors (herein referred to as reprogramming factors) in the somatic cell. iPSCs can be generated using fetal, postnatal, newborn, juvenile, or adult somatic cells. In some instances, factors that can be used to reprogram somatic cells to pluripotent stem cells include, for example, OCT4 (OCT3/4), SOX2, c-MYC, and KLF4, NANOG, and LIN28. In some instances, somatic cells may be reprogrammed by expressing at least two reprogramming factors, at least three reprogramming factors, or at least four reprogramming factors, to reprogram a somatic cell to a pluripotent stem cell. The cells may be reprogrammed by introducing reprogramming factors using vectors, including, for example, lentivirus, retrovirus, adenovirus, and Sendai virus vectors. Alternatively, non-viral techniques for introducing reprogramming factors include, for example, mRNA transfection, miRNA infection/transfection, PiggyBac, minicircle vectors, and episomal plasmids. iPSCs may also be generated by, for example, using CRISPR-Cas9-based techniques, to introduce reprogramming factors, or to activate endogenous programming genes.

As used herein, “embryonic stem cells” are embryonic cells derived from embryo tissue, preferably the inner cell mass of blastocysts or morulae, optionally that have been serially passaged as cell lines. The term includes cells isolated from one or more blastomeres of an embryo, preferably without destroying the remainder of the embryo. The term also includes cells produced by somatic cell nuclear transfer. ESCs can be produced or derived from a zygote, blastomere, or blastocyst-staged mammalian embryo produced by the fusion of a sperm and egg cell, nuclear transfer, or parthenogenesis, for example. Human ESCs include, without limitation, MAO1, MAO9, ACT-4, No. 3, H1, H7, H9, H14 and ACT30 embryonic stem cells. Exemplary pluripotent stem cells include embryonic stem cells derived from the inner cell mass (ICM) of blastocyst stage embryos, as well as embryonic stem cells derived from one or more blastomeres of a cleavage stage or morula stage embryo. These embryonic stem cells can be generated from embryonic material produced by fertilization or by asexual means, including somatic cell nuclear transfer (SCNT), parthenogenesis, and androgenesis. PSCs alone cannot develop into a fetal or adult animal when transplanted in utero because they lack the potential to contribute to all extraembryonic tissue (e.g., placenta in vivo or trophoblast in vitro).

As used herein, the term “progenitor cell” refers to a descendant of a stem cell which is capable of further differentiation into one or more kinds of specialized cells, but which cannot divide and reproduce indefinitely. That is, unlike stem cells (which possess an unlimited capacity for self-renewal), progenitor cells possess only a limited capacity for self-renewal. Progenitor cells may be multipotent, oligopotent, or unipotent, and are typically classified according to the types of specialized cells they can differentiate into. For instance, a “cardiomyocyte progenitor cell” is a progenitor cell derived from a stem cell that has the capacity to differentiate into a cardiomyocyte. Similarly, “cardiac progenitor cells” may differentiate into multiple specialized cells constituting cardiac tissue, including, for example, cardiomyocytes, smooth muscle cells, and endothelial cells. Additionally, a “cardiovascular progenitor cell” has the capacity to differentiate into, for example, cells of cardiac and vascular lineages.

As used herein, “expand” or “proliferate” may refer to a process by which the number of cells in a cell culture is increased due to cell division.

“Multipotent” implies that a cell is capable, through its progeny, of giving rise to several different cell types found in an adult animal.

“Pluripotent” implies that a cell is capable, through its progeny, of giving rise to all the cell types that comprise the adult animal, including the germ cells. Embryonic stem cells, induced pluripotent stem cells, and embryonic germ cells are pluripotent cells under this definition.

The term “autologous cells” as used herein refers to donor cells that are genetically identical with the recipient.

As used herein, the term “allogeneic cells” refers to cells derived from a different, genetically non-identical, individual of the same species.

The term “totipotent” as used herein can refer to a cell that gives rise to a live born animal. The term “totipotent” can also refer to a cell that gives rise to all of the cells in a particular animal. A totipotent cell can give rise to all of the cells of an animal when it is utilized in a procedure for developing an embryo from one or more nuclear transfer steps.

As used herein, the term “extracellular vesicles” collectively refers to biological nanoparticles derived from cells, and examples thereof include exosomes, ectosomes, exovesicles, microparticles, microvesicles, nanovesicles, blebbing vesicles, budding vesicles, exosome-like vesicles, matrix vesicles, membrane vesicles, shedding vesicles, membrane particles, shedding microvesicles, oncosomes, exomeres, and apoptotic bodies, but are not limited thereto.

Extracellular vesicles can be categorized, for example, according to size. For instance, as used herein, the term “small extracellular vesicle” refers to extracellular vesicles having a diameter of between about 50-200 nm. In contrast, extracellular vesicles having a diameter of more than about 200 nm, but less than 400 nm, may be referred to as “medium extracellular vesicles,” and extracellular vesicles having a diameter of more than about 400 nm may be referred to as “large extracellular vesicles.” As used herein, the term “small extracellular vesicle fraction” (“sEV”) refers to a part, extract, or fraction, of secretome or conditioned medium, that is concentrated and/or enriched for small extracellular vesicles having a diameter of between about 50-200 nm. Such concentration and/or enrichment may be obtained using one or more of the purification, isolation, concentration, and/or enrichment, techniques disclosed herein. In some alternative embodiments herein, enrichment may not be performed, may not be achieved, or may not be possible.

The term “exosome” as used herein refers to an extracellular vesicle that is released from a cell upon fusion of the multivesicular body (MVB) (an intermediate endocytic compartment) with the plasma membrane.

“Exosome-like vesicles,” which have a common origin with exosomes, are typically described as having size and sedimentation properties that distinguish them from exosomes and, particularly, as lacking lipid raft microdomains. “Ectosomes,” as used herein, are typically neutrophil- or monocyte-derived microvesicles.

“Microparticles” as used herein are typically about 100-1000 nm in diameter and originate from the plasma membrane. “Extracellular membranous structures” also include linear or folded membrane fragments, e.g., from necrotic death, as well as membranous structures from other cellular sources, including secreted lysosomes and nanotubes.

As used herein, “apoptotic blebs or bodies” are typically about 1 to 5 μm in diameter and are released as blebs of cells undergoing apoptosis, i.e., diseased, unwanted and/or aberrant cells.

Within the class of extracellular vesicles, important components are “exosomes” themselves, which may be between about 40 to 50 nm and about 200 nm in diameter and being membranous vesicles, i.e., vesicles surrounded by a phospholipid bilayer, of endocytic origin, which result from exocytic fusion, or “exocytosis” of multivesicular bodies (MVBs). In some cases, exosomes can be between about 40 to 50 nm up to about 200 nm in diameter, such as being from 60 nm to 180 nm.

As used herein, the terms “secretome” and “secretome composition” interchangeably refer to one or more molecules and/or biological factors that are secreted by cells into the extracellular space (such as into a culture medium). A secretome or secretome composition may include, without limitation, extracellular vesicles (e.g., exosomes, microparticles, etc.), proteins, nucleic acids, cytokines, and/or other molecules secreted by cells into the extracellular space (such as into a culture medium). A secretome or secretome composition may be left unpurified or further processed (for example, components of a secretome or secretome composition may be present within culture medium, such as in a conditioned medium; or alternatively, components of a secretome or secretome composition may be purified, isolated, and/or enriched, from a culture medium or extract, part, or fraction thereof). A secretome or secretome composition may further comprise one or more substances that are not secreted from a cell (e.g., culture media, additives, nutrients, etc.). Alternatively, a secretome or secretome composition does not comprise one or more substances (or comprises only trace amounts thereof) that are not secreted from a cell (e.g., culture media, additives, nutrients, etc.).

As used herein, the term “conditioned medium” refers to a culture medium (or extract, part, or fraction thereof) in which one or more cells of interest have been cultured. Preferably, conditioned medium is separated from the cultured cells before use and/or further processing. The culturing of cells in culture medium may result in the secretion and/or accumulation of one or more molecules and/or biological factors (which may include, without limitation, extracellular vesicles (e.g., exosomes, microparticles, etc.), proteins, nucleic acids, cytokines, and/or other molecules secreted by cells into the extracellular space); the medium containing the one or more molecules and/or biological factors is a conditioned medium. Examples of methods of preparing conditioned media have been described in, for example, U.S. Pat. No. 6,372,494, which is incorporated by reference herein in its entirety.

As used herein, the term “cell culture” refers to cells grown under controlled condition(s) outside the natural environment of the cells. For instance, cells can be propagated completely outside of their natural environment (in vitro), or can be removed from their natural environment and the cultured (ex vivo). During cell culture, cells may survive in a non-replicative state, or may replicate and grow in number, depending on, for example, the specific culture media, the culture conditions, and the type of cells. An in vitro environment can be any medium known in the art that is suitable for maintaining cells in vitro, such as suitable liquid media or agar, for example.

The term “cell line” as used herein can refer to cultured cells that can be passaged at least one time without terminating.

The term “suspension” as used herein can refer to cell culture conditions in which cells are not attached to a solid support. Cells proliferating in suspension can be stirred while proliferating using an apparatus well known to those skilled in the art.

The term “monolayer” as used herein can refer to cells that are attached to a solid support while proliferating in suitable culture conditions. A small portion of cells proliferating in a monolayer under suitable growth conditions may be attached to cells in the monolayer but not to the solid support.

The term “plated” or “plating” as used herein in reference to cells can refer to establishing cell cultures in vitro. For example, cells can be diluted in cell culture media and then added to a cell culture plate, dish, or flask. Cell culture plates are commonly known to a person of ordinary skill in the art. Cells may be plated at a variety of concentrations and/or cell densities.

The term “cell plating” can also extend to the term “cell passaging.” Cells can be passaged using cell culture techniques well known to those skilled in the art. The term “cell passaging” can refer to a technique that involves the steps of (1) releasing cells from a solid support or substrate and disassociation of these cells, and (2) diluting the cells in media suitable for further cell proliferation. Cell passaging may also refer to removing a portion of liquid medium containing cultured cells and adding liquid medium to the original culture vessel to dilute the cells and allow further cell proliferation. In addition, cells may also be added to a new culture vessel that has been supplemented with medium suitable for further cell proliferation.

As used herein, the terms “culture medium,” “growth medium” or “medium” are used interchangeably and refer to a composition that is intended to support the growth and survival of organisms. While culture media is often in liquid form, other physical forms may be used, such as, for example, a solid, semi-solid, gel, suspension, and the like.

As used herein, the term “serum-free,” in the context of a culture medium or growth medium, refers to a culture or growth medium in which serum is absent. Serum typically refers to the liquid component of clotted blood, after the clotting factors (e.g., fibrinogen and prothrombin) have been removed by clot formation. Serum, such as fetal bovine serum, is routinely used in the art as a component of cell culture media, as the various proteins and growth factors therein are particularly useful for the survival, growth, and division of cells.

As used herein, the term “basal medium” refers to an unsupplemented synthetic medium that may contain buffers, one or more carbon sources, amino acids, and salts. Depending on the application, basal medium may be supplemented with growth factors and supplements, including, but not limited to, additional buffering agents, amino acids, antibiotics, proteins, and growth factors useful, for instance, for promoting growth, or maintaining or changing differentiation status, of particular cell types (e.g., fibroblast growth factor-basic (bFGF), also known as fibroblast growth factor 2 (FGF-2)).

As used herein, the terms “wild-type,” “naturally occurring,” and “unmodified” are used herein to mean the typical (or most common) form, appearance, phenotype, or strain existing in nature; for example, the typical form of cells, organisms, polynucleotides, proteins, macromolecular complexes, genes, RNAs, DNAs, or genomes as they occur in, and can be isolated from, a source in nature. The wild-type form, appearance, phenotype, or strain serve as the original parent before an intentional modification. Thus, mutant, variant, engineered, recombinant, and modified forms are not wild-type forms.

As used herein, the term “isolated” refers to material removed from its original environment, and is thus altered “by the hand of man” from its natural state.

As used herein, the term “enriched” means to selectively concentrate or increase the amount of one or more components in a composition, with respect to one or more other components. For instance, enrichment may include reducing or decreasing the amount of (e.g., removing or eliminating) unwanted materials; and/or may include specifically selecting or isolating desirable materials from a composition.

The terms “engineered,” “genetically engineered,” “genetically modified,” “recombinant,” “modified,” “non-naturally occurring,” and “non-native” indicate intentional human manipulation of the genome of an organism or cell. The terms encompass methods of genomic modification that include genomic editing, as defined herein, as well as techniques that alter gene expression or inactivation, enzyme engineering, directed evolution, knowledge-based design, random mutagenesis methods, gene shuffling, codon optimization, and the like. Methods for genetic engineering are known in the art.

As used herein, the terms “nucleic acid sequence,” “nucleotide sequence,” and “oligonucleotide” all refer to polymeric forms of nucleotides. As used herein, the term “polynucleotide” refers to a polymeric form of nucleotides that, when in linear form, has one 5′ end and one 3′ end, and can comprise one or more nucleic acid sequences. The nucleotides may be deoxyribonucleotides (DNA), ribonucleotides (RNA), analogs thereof, or combinations thereof, and may be of any length. Polynucleotides may perform any function and may have various secondary and tertiary structures. The terms encompass known analogs of natural nucleotides and nucleotides that are modified in the base, sugar, and/or phosphate moieties. Analogs of a particular nucleotide have the same base-pairing specificity (e.g., an analog of A base pairs with T). A polynucleotide may comprise one modified nucleotide or multiple modified nucleotides. Examples of modified nucleotides include fluorinated nucleotides, methylated nucleotides, and nucleotide analogs. Nucleotide structure may be modified before or after a polymer is assembled. Following polymerization, polynucleotides may be additionally modified via, for example, conjugation with a labeling component or target binding component. A nucleotide sequence may incorporate non-nucleotide components. The terms also encompass nucleic acids comprising modified backbone residues or linkages, that are synthetic, naturally occurring, and/or non-naturally occurring, and have similar binding properties as a reference polynucleotide (e.g., DNA or RNA). Examples of such analogs include, but are not limited to, phosphorothioates, phosphoramidates, methyl phosphonates, chiral-methyl phosphonates, 2-O-methyl ribonucleotides, peptide-nucleic acids (PNAs), Locked Nucleic Acid (LNA™) (Exiqon, Inc., Woburn, MA) nucleosides, glycol nucleic acid, bridged nucleic acids, and morpholino structures. Peptide-nucleic acids (PNAs) are synthetic homologs of nucleic acids wherein the polynucleotide phosphate-sugar backbone is replaced by a flexible pseudo-peptide polymer. Nucleobases are linked to the polymer. PNAs have the capacity to hybridize with high affinity and specificity to complementary sequences of RNA and DNA. Polynucleotide sequences are displayed herein in the conventional 5′ to 3′ orientation unless otherwise indicated.

As used herein, “sequence identity” generally refers to the percent identity of nucleotide bases or amino acids comparing a first polynucleotide or polypeptide to a second polynucleotide or polypeptide using algorithms having various weighting parameters. Sequence identity between two polynucleotides or two polypeptides can be determined using sequence alignment by various methods and computer programs (e.g., Exonerate, BLAST, CS-BLAST, FASTA, HMMER, L-ALIGN, and the like) available through the worldwide web at sites including, but not limited to, GENBANK (www.ncbi.nlm.nih.gov/genbank/) and EMBL-EBI (www.ebi.ac.uk.). Sequence identity between two polynucleotides or two polypeptide sequences is generally calculated using the standard default parameters of the various methods or computer programs. A high degree of sequence identity between two polynucleotides or two polypeptides is often between about 90% identity and 100% identity over the length of the reference polynucleotide or polypeptide or query sequence, for example, about 90% identity or higher, about 91% identity or higher, about 92% identity or higher, about 93% identity or higher, about 94% identity or higher, about 95% identity or higher, about 96% identity or higher, about 97% identity or higher, about 98% identity or higher, or about 99% identity or higher, over the length of the reference polynucleotide or polypeptide or query sequence. Sequence identity can also be calculated for the overlapping region of two sequences where only a portion of the two sequences can be aligned.

A moderate degree of sequence identity between two polynucleotides or two polypeptides is often between about 80% identity to about 90% identity over the length of the reference polynucleotide or polypeptide or query sequence, for example, about 80% identity or higher, about 81% identity or higher, about 82% identity or higher, about 83% identity or higher, about 84% identity or higher, about 85% identity or higher, about 86% identity or higher, about 87% identity or higher, about 88% identity or higher, or about 89% identity or higher, but less than 90%, over the length of the reference polynucleotide or polypeptide or query sequence.

A low degree of sequence identity between two polynucleotides or two polypeptides is often between about 50% identity and 75% identity over the length of the reference polynucleotide or polypeptide or query sequence, for example, about 50% identity or higher, about 60% identity or higher, about 70% identity or higher, but less than 75% identity, over the length of the reference polynucleotide or polypeptide or query sequence.

As used herein, “binding” refers to a non-covalent interaction between macromolecules (e.g., between a protein and a polynucleotide, between a polynucleotide and a polynucleotide, or between a protein and a protein, and the like). Such non-covalent interaction is also referred to as “associating” or “interacting” (e.g., if a first macromolecule interacts with a second macromolecule, the first macromolecule binds to second macromolecule in a non-covalent manner). Some portions of a binding interaction may be sequence-specific (the terms “sequence-specific binding,” “sequence-specifically bind,” “site-specific binding,” and “site specifically binds” are used interchangeably herein). Binding interactions can be characterized by a dissociation constant (Kd). “Binding affinity” refers to the strength of the binding interaction. An increased binding affinity is correlated with a lower Kd.

“Gene” as used herein refers to a polynucleotide sequence comprising exons and related regulatory sequences. A gene may further comprise introns and/or untranslated regions (UTRs).

As used herein, “expression” refers to transcription of a polynucleotide from a DNA template, resulting in, for example, a messenger RNA (mRNA) or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene products.” Expression may include splicing the mRNA in a eukaryotic cell, if the polynucleotide is derived from genomic DNA.

A “coding sequence” or a sequence that “encodes” a selected polypeptide, is a nucleic acid molecule that is transcribed (in the case of DNA) and translated (in the case of mRNA) into a polypeptide in vitro or in vivo when placed under the control of appropriate regulatory sequences. The boundaries of the coding sequence are determined by a start codon at the 5′ terminus and a translation stop codon at the 3′ terminus. A transcription termination sequence may be located 3′ to the coding sequence.

As used herein, a “different” or “altered” level of, for example, a characteristic or property, is a difference that is measurably different, and preferably, statistically significant (for example, not attributable to the standard error of the assay). In some embodiments, a difference, e.g., as compared to a control or reference sample, may be, for example, a greater than 10% difference, a greater than 20% difference, a greater than 30% difference, a greater than 40% difference, a greater than 50% difference, a greater than 60% difference, a greater than 70% difference, a greater than 80% difference, a greater than 90% difference, a greater than 2-fold difference; a greater than 5-fold difference; a greater than 10-fold difference; a greater than 20-fold difference; a greater than 50-fold difference; a greater than 75-fold difference; a greater than 100-fold difference; a greater than 250-fold difference; a greater than 500-fold difference; a greater than 750-fold difference; or a greater than 1,000-fold difference, for example.

As used herein, the term “between” is inclusive of end values in a given range (e.g., between about 1 and about 50 nucleotides in length includes 1 nucleotide and 50 nucleotides).

As used herein, the term “amino acid” refers to natural and synthetic (unnatural) amino acids, including amino acid analogs, modified amino acids, peptidomimetics, glycine, and D or L optical isomers.

As used herein, the terms “peptide,” “polypeptide,” and “protein” are interchangeable and refer to polymers of amino acids. A polypeptide may be of any length. It may be branched or linear, it may be interrupted by non-amino acids, and it may comprise modified amino acids. The terms also refer to an amino acid polymer that has been modified through, for example, acetylation, disulfide bond formation, glycosylation, lipidation, phosphorylation, pegylation, biotinylation, cross-linking, and/or conjugation (e.g., with a labeling component or ligand). Polypeptide sequences are displayed herein in the conventional N-terminal to C-terminal orientation, unless otherwise indicated. Polypeptides and polynucleotides can be made using routine techniques in the field of molecular biology.

A “moiety” as used herein refers to a portion of a molecule. A moiety can be a functional group or describe a portion of a molecule with multiple functional groups (e.g., that share common structural aspects). The terms “moiety” and “functional group” are typically used interchangeably; however, a “functional group” can more specifically refer to a portion of a molecule that comprises some common chemical behavior. “Moiety” is often used as a structural description.

The terms “effective amount” or “therapeutically effective amount” of a composition or agent, such as a therapeutic composition as provided herein, refers to a sufficient amount of the composition or agent to provide the desired response. Such responses will depend on the particular disease in question.

“Transformation” as used herein refers to the insertion of an exogenous polynucleotide into a host cell, irrespective of the method used for insertion. For example, transformation can be by direct uptake, transfection, infection, and the like. The exogenous polynucleotide may be maintained as a nonintegrated vector, for example, an episome, or, alternatively, may be integrated into the host genome.

As used herein, the term “hypoxia” or “hypoxic” refers to a condition where the oxygen (02) concentration is below atmospheric 02 concentration (typically 20-21%). In some embodiments, hypoxia refers to a condition with an 02 concentration that is between 0% and 19%, between 2% and 18%, between 3% and 17%, between 4% and 16%, between 5% and 15%, between 5% and 10%, or less than 10%, less than 9%, less than 8%, less than 7%, less than 6%, less than 5%, less than 4%, less than 3%, less than 2%, or less than 1%.

As used herein, the term “normoxia” refers to a normal atmospheric concentration of oxygen, typically around 20% to 21% 02.

Generation of Progenitor Cells from Stem Cells

The present disclosure relates, in part, to methods for generating a secretome containing extracellular vesicles (EVs) from progenitor cells. In certain embodiments herein, progenitor cells may be isolated from a subject or tissue, and used in the methods of the present disclosure. In other embodiments, progenitor cells may be generated from pluripotent stem cells, such as from embryonic stem (ES) cells or induced pluripotent stem cells (iPSCs).

Generation of iPSC Cells

iPSC cells may be obtained from, for example, somatic cells, including human somatic cells. The somatic cell may be derived from a human or non-human animal, including, for example, humans and other primates, including non-human primates, such as rhesus macaques, chimpanzees, and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys, and other gallinaceous birds, ducks, and geese; and the like.

In some embodiments, the somatic cell is selected from keratinizing epithelial cells, mucosal epithelial cells, exocrine gland epithelial cells, endocrine cells, liver cells, epithelial cells, endothelial cells, fibroblasts, muscle cells, cells of the blood and the immune system, cells of the nervous system including nerve cells and glial cells, pigment cells, and progenitor cells, including hematopoietic stem cells. The somatic cell may be fully differentiated (specialized), or may be less than fully differentiated. For instance, undifferentiated progenitor cells that are not PSCs, including somatic stem cells, and finally differentiated mature cells, can be used. The somatic cell may be from an animal of any age, including adult and fetal cells.

The somatic cell may be of mammalian origin. Allogeneic or autologous stem cells can be used, if for example, the secretome (or extracellular vesicles) from a progenitor cell thereof is used for administration in vivo. In some embodiments, iPSCs are not MHC-/HLA-matched to a subject. In some embodiments, iPSCs are MHC-/HLA-matched to a subject. In embodiments, for example, where iPSCs are to be used to produce PSC-derived progenitor cells (to obtain a secretome, or extracellular vesicles, for therapeutic use in a subject), somatic cells may be obtained from the subject to be treated, or from another subject with the same or substantially the same HLA type as that of the subject. Somatic cells can be cultured before nuclear reprogramming, or can be reprogrammed without culturing after isolation, for example.

To introduce reprogramming factors into somatic cells, for example, viral vectors may be used, including, e.g., vectors from viruses such as SV40, adenovirus, vaccinia virus, adeno-associated virus, herpes viruses including HSV and EBV, Sindbis viruses, alphaviruses, human herpesvirus vectors (HHV) such as HHV-6 and HHV-7, and retroviruses. Lentiviruses include, but are not limited to, Human Immunodeficiency Virus type 1 (HIV-1), Human Immunodeficiency Virus type 2 (HIV-2), Simian Immunodeficiency Virus (SIV), Feline Immunodeficiency Virus (FIV), Equine Infectious Anaemia Virus (EIAV), Bovine Immunodeficiency Virus (BIV), Visna Virus of sheep (VISNA) and Caprine Arthritis-Encephalitis Virus (CAEV). Lentiviral vectors are capable of infecting non-dividing cells and can be used for both in vivo and in vitro gene transfer and expression of nucleic acid sequences. A viral vector can be targeted to a specific cell type by linkage of a viral protein, such as an envelope protein, to a binding agent, such as an antibody, or a particular ligand (for targeting to, for instance, a receptor or protein on or within a particular cell type).

In some embodiments, a viral vector, such as a lentiviral vector, can integrate into the genome of the host cell. The genetic material thus transferred is then transcribed and possibly translated into proteins inside the host cell. In other embodiments, viral vectors are used that do not integrate into the genome of a host cell.

A viral gene delivery system can be an RNA-based or DNA-based viral vector. An episomal gene delivery system can be a plasmid, an Epstein-Barr virus (EBV)-based episomal vector, a yeast-based vector, an adenovirus-based vector, a simian virus 40 (SV40)-based episomal vector, a bovine papilloma virus (BPV)-based vector, or a lentiviral vector, for example.

Somatic cells can be reprogrammed to produce induced pluripotent stem cells (iPSCs) using methods known to one of skill in the art. One of skill in the art can readily produce induced pluripotent stem cells, see for example, Published U.S. Patent Application No. 2009/0246875, Published U.S. Patent Application No. 2010/0210014; Published U.S. Patent Application No. 2012/0276636; U.S. Pat. Nos. 8,058,065; 8,129,187; and 8,268,620, all of which are incorporated herein by reference.

Generally, reprogramming factors which can be used to create induced pluripotent stem cells, either singly, in combination, or as fusions with transactivation domains, include, but are not limited to, one or more of the following genes: Oct4 (Oct3/4, Pou5f1), Sox (e.g., Sox1, Sox2, Sox3, Sox18, or Sox15), Klf (e.g., Klf4, Klf1, Klf3, Klf2 or Klf5), Myc (e.g., c-myc, N-myc or L-myc), nanog, or LIN28. As examples of sequences for these genes and proteins, the following accession numbers are provided: Mouse MyoD: M84918, NM_010866; Mouse Oct4 (POU5F1): NM_013633; Mouse Sox2: NM_011443; Mouse Klf4: NM_010637; Mouse c-Myc: NM_001177352, NM_001177353, NM_001177354 Mouse Nanog: NM_028016; Mouse Lin28: NM_145833: Human MyoD: NM_002478; Human Oct4 (POU5F1): NM_002701, NM_203289, NM_001173531; Human Sox2: NM_003106; Human Klf4: NM_004235; Human c-Myc: NM_002467; Human Nanog: NM_024865; and/or Human Lin28: NM_024674. Also contemplated are sequences similar thereto, including those having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity. In some embodiments, at least three, or at least four, of Klf4, c-Myc, Oct3/4, Sox2, Nanog, and Lin28 are utilized. In other embodiments, Oct3/4, Sox2, c-Myc and Klf4 is utilized.

Exemplary reprogramming factors for the production of iPSCs include (1) Oct3/4, Klf4, Sox2, L-Myc (Sox2 can be replaced with Sox1, Sox3, Sox15, Sox17 or Sox18; Klf4 is replaceable with Klf1, Klf2 or Klf5); (2) Oct3/4, Klf4, Sox2, L-Myc, TERT, SV40 Large T antigen (SV40LT); (3) Oct3/4, Klf4, Sox2, L-Myc, TERT, human papilloma virus (HPV)16 E6; (4) Oct3/4, Klf4, Sox2, L-Myc, TERT, HPV16 E7 (5) Oct3/4, Klf4, Sox2, L-Myc, TERT, HPV16 E6, HPV16 E7; (6) Oct3/4, Klf4, Sox2, L-Myc, TERT, Bmi1; (7) Oct3/4, Klf4, Sox2, L-Myc, Lin28; (8) Oct3/4, Klf4, Sox2, L-Myc, Lin28, SV40LT; (9) Oct3/4, Klf4, Sox2, L-Myc, Lin28, TERT, SV40LT; (10) Oct3/4, Klf4, Sox2, L-Myc, SV40LT; (11) Oct3/4, Esrrb, Sox2, L-Myc (Esrrb is replaceable with Esrrg); (12) Oct3/4, Klf4, Sox2; (13) Oct3/4, Klf4, Sox2, TERT, SV40LT; (14) Oct3/4, Klf4, Sox2, TERT, HPV16 E6; (15) Oct3/4, Klf4, Sox2, TERT, HPV16 E7; (16) Oct3/4, Klf4, Sox2, TERT, HPV16 E6, HPV16 E7; (17) Oct3/4, Klf4, Sox2, TERT, Bmi1; (18) Oct3/4, Klf4, Sox2, Lin28 (19) Oct3/4, Klf4, Sox2, Lin28, SV40LT; (20) Oct3/4, Klf4, Sox2, Lin28, TERT, SV40LT; (21) Oct3/4, Klf4, Sox2, SV40LT; or (22) Oct3/4, Esrrb, Sox2 (Esrrb is replaceable with Esrrg).

iPSCs typically display the characteristic morphology of human embryonic stem cells (hESCs), and express the pluripotency factor, NANOG. Embryonic stem cell specific surface antigens (SSEA-3, SSEA-4, TRA1-60, TRA1-81) may also be used to identify fully reprogrammed human cells. Additionally, at a functional level, PSCs, such as ESCs and iPSCs, also demonstrate the ability to differentiate into lineages from all three embryonic germ layers, and form teratomas in vivo (e.g., in SCID mice).

Differentiating PSCs to Generate Progenitor Cells

The present disclosure further contemplates differentiating PSCs, including ESCs and iPSCs, into progenitor cells. Such progenitor cells can then be used to produce a secretome (and extracellular vesicles) of the present disclosure.

Progenitor cells of the present disclosure include, for example, hematopoietic progenitor cells, myeloid progenitor cells, neural progenitor cells; pancreatic progenitor cells, cardiac progenitor cells, cardiomyocyte progenitor cells, cardiovascular progenitor cells, renal progenitor cells, skeletal myoblasts, satellite cells, intermediate progenitor cells formed in the subventricular zone, radial glial cells, bone marrow stromal cells, periosteum cells, endothelial progenitor cells, blast cells, boundary caop cells, and mesenchymal stem cells. Methods for differentiating pluripotent stem cells to progenitor cells, and for culturing and maintaining progenitor cells, are known in the art, such as those described in U.S. Provisional Patent Application No. 63/243,606 entitled “Methods for the Production of Committed Cardiac Progenitor Cells,” which is incorporated by reference herein in its entirety.

Culturing of Progenitor Cells for Secretome/Extracellular Vesicle Production

The present disclosure encompasses the culturing of progenitor cells for secretome/extracellular vesicle production under GMP-ready and/or GMP-compatible conditions, to produce, e.g., GMP-ready and/or GMP-compatible products. The present disclosure also encompasses the culturing of progenitor cells for secretome/extracellular vesicle production under non-GMP-ready and/or non-GMP-compatible conditions, to produce, e.g., non-GMP-ready and/or non-GMP-compatible products.

In methods for generating secretomes or extracellular vesicles of the present disclosure, progenitor cells are typically subjected to two or more culturing steps in a serum-free culture medium.

In a first culturing step, one or more progenitor cells are cultured in a first serum-free culture medium that comprises basal medium, human serum albumin, and one or more growth factors. This first serum-free culture medium is then replaced with a second serum-free culture medium that comprises basal medium, but does not comprise human serum albumin or the one or more growth factors. In a second culturing step, the one or more progenitor cells are then cultured in the second serum-free culture medium. Following the second culturing step, the second serum-free culture medium is recovered, to thereby obtain conditioned medium containing the secretome of the one or more progenitor cells.

The one or more progenitor cells can be, for example, progenitor cells that have recently been isolated or differentiated (e.g., from stem cells). Alternatively, in some embodiments, progenitor cells that have previously been refrigerated, frozen, and/or cryopreserved, may be used in the culturing methods of the present disclosure. In some embodiments, progenitor cells are thawed from a cryopreserved state (e.g., −80° C. or colder) before use. In some embodiments thereof, the cells are thawed in a thawing medium. In some embodiments, the thawing medium may comprise a liquid medium (e.g., alpha-MEM, STEMdiffrM Cardiomyocyte Support Medium (StemCell, Ref: 05027)) containing one or more supplements. In some embodiments, the supplement in the thawing medium may be one or more of a carbon source (e.g., glucose), an albumin, B-27, insulin, FGF-2, FGF, and an antibiotic (e.g., gentamicin). In some embodiments, the cells may be thawed in a thawing device, such as, for example, a water bath or a water-free thawing system (e.g., ThawSTAR™ Automated Thawing System, Biolife Solutions®). Cells may be thawed, for example, within a tube or bottle (e.g., plastic, glass), or bag (e.g., an Ethyl Vinyl Acetate (EVA) bag), such as a 500-1000 mL volume bag (e.g., Corning, Refs: 91-200-41, 91-200-42).

The one or more growth factors may be selected based on the type of progenitor cell, for example. In some embodiments, the one or more growth factors may be selected from Adrenomedullin, Angiopoietin, Autocrine motility factor, Bone morphogenetic proteins (BMPs), Ciliary neurotrophic factor (CNTF), Leukemia inhibitory factor (LIF), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor 1 (FGF-1), Fibroblast growth factor 2 (FGF-2), Fibroblast growth factor 3 (FGF-3), Fibroblast growth factor 4 (FGF-4), Fibroblast growth factor 5 (FGF-5), Fibroblast growth factor 6 (FGF-6), Fibroblast growth factor 7 (FGF-7), Fibroblast growth factor 8 (FGF-8), Fibroblast growth factor 9 (FGF-9), Fibroblast growth factor 10 (FGF-10), Fibroblast growth factor 11 (FGF-11), Fibroblast growth factor 12 (FGF-12), Fibroblast growth factor 13 (FGF-13), Fibroblast growth factor 14 (FGF-14), Fibroblast growth factor 15 (FGF-15), Fibroblast growth factor 16 (FGF-16), Fibroblast growth factor 17 (FGF-17), Fibroblast growth factor 18 (FGF-18), Fibroblast growth factor 19 (FGF-19), Fibroblast growth factor 20 (FGF-20), Fibroblast growth factor 21 (FGF-21), Fibroblast growth factor 22 (FG-F22), Fibroblast growth factor 23 (FGF-23), Foetal Bovine Somatotrophin (FBS), Glial cell line-derived neurotrophic factor (GDNF), Neurturin, Persephin, Artemin, Growth differentiation factor-9 (GDF-9), Hepatocyte growth factor (HGF), Hepatoma-derived growth factor (HDGF), Insulin, Insulin-like growth factor-1 (IGF-1), Insulin-like growth factor-2 (IGF-2), IL-1, IL-2, IL-3, IL-4, IL-5, IL-6, IL-7, Keratinocyte growth factor (KGF), Migration-stimulating factor (MSF), Macrophage-stimulating protein (MSP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS), T-cell growth factor (TCGF), Thrombopoietin (TPO), Transforming growth factor alpha (TGF-α), Transforming growth factor beta (TGF-β), Tumor necrosis factor-alpha (TNF-α), and Vascular endothelial growth factor (VEGF).

The amount of growth factor may be adjusted depending on the desired culture conditions and/or need. In some embodiments, the one or more growth factors may each independently be present in an amount from 0.001 μg/mL-1000 μg/mL, in an amount from 0.01 μg/mL-100 μg/mL, in an amount from 0.1 μg/mL-10 μg/mL, in an amount from 0.05 μg/mL-5 μg/mL, in an amount from 0.5 μg/mL-2.5 μg/mL, or in an amount of about 0.5 μg/mL, about 1 μg/mL, about 2 μg/mL, about 3 μg/mL, about 4 μg/mL or about 5 μg/mL.

In some embodiments, the one or more growth factors comprise FGF-2. In some embodiments, the one or more growth factors consist of FGF-2.

The basal medium may be any basal culture medium suitable for the cell type to be cultured, including, for example, Dulbecco's Modified Eagle's Medium (DMEM), DMEM F12 medium, Eagle's Minimum Essential Medium (MEM), α-MEM, F-12K medium, Iscove's Modified Dulbecco's Medium, Knockout DMEM, or RPMI-1640 medium, or variants, combinations, or modifications thereof.

Additional supplements can also be added to the basal medium to supply the cells with trace elements for optimal growth and expansion. Such supplements include, for example, insulin, transferrin, sodium selenium, Hanks' Balanced Salt Solution, Earle's Salt Solution, antioxidant supplements, MCDB-201, phosphate buffered saline (PBS), N-2-hydroxyethylpiperazine-N′-ethanesulfonic acid (HEPES), nicotinamide, ascorbic acid and/or ascorbic acid-2-phosphate, as well as additional amino acids, and combinations thereof. Such amino acids include, but are not limited to, L-alanine, L-arginine, L-aspartic acid, L-asparagine, L-cysteine, L-cysteine, L-glutamic acid, L-glutamine, L-glycine, L-histidine, L-inositol, L-isoleucine, L-leucine, L-lysine, L-methionine, L-phenylalanine, L-proline, L-serine, L-threonine, L-tryptophan, L-tyrosine, and L-valine.

Optionally, hormones can also be used in cell culture and include, but are not limited to, D-aldosterone, diethylstilbestrol (DES), dexamethasone, beta-estradiol, hydrocortisone, insulin, prolactin, progesterone, somatostatin/human growth hormone (HGH), thyrotropin, thyroxine, and L-thyronine. Beta-mercaptoethanol can also be supplemented in cell culture media.

Lipids and lipid carriers can also be used to supplement cell culture media, depending on the type of cell. Such lipids and carriers can include, but are not limited to, cyclodextrin, cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others.

In certain embodiments, an albumin, such as human serum albumin, is present in the first serum-free culture medium. The albumin, including human serum albumin, may be, for example, isolated, synthetic, recombinant, and/or modified. The amount of albumin may be adjusted depending on the desired culture conditions and/or need. In some embodiments, the albumin may be present in an amount from 0.1 μg/mL-50 mg/mL, in an amount from 1 μg/mL-25 mg/mL, in an amount from 10 μg/mL-20 mg/mL, in an amount from 100 μg/mL-10 mg/mL, in an amount from 0.5 mg/mL-5 mg/mL, in an amount from 1 mg/mL-3 mg/mL, or in an amount of about 0.5 mg/mL, 1 mg/mL, 2 mg/mL, 3 mg/mL, 4 mg/mL or 5 mg/mL.

In some embodiments, the serum-free media further comprises one or more selected from the group consisting of: glutamine; biotin; DL alpha tocopherol acetate; DL alpha-tocopherol; vitamin A; catalase; insulin; transferrin; superoxide dismutase; corticosterone; D-galactose; ethanolamine, glutathione; L-carnitine; linoleic acid; progesterone; putrescine; sodium selenite; triodo-I-thyronine; an amino acid; sodium pyruvate; lipoic acid; vitamin B12; nucleosides; and ascorbic acid.

The basal medium may also be supplemented with one or more carbon sources. The one or more carbon sources may be selected from, for example, carbon sources such as glycerol, glucose, galactose, sucrose, fructose, mannose, lactose, or maltose.

The first and second culturing steps may be performed for differing lengths of time. For instance, the first and second culturing steps may each independently be performed for a period of 6-96 hours, 12-72 hours, 36-60 hours, 42-56 hours, or for about 12 hours, about 18 hours, about 24 hours, about 30 hours, about 36 hours, about 42 hours, about 48 hours, about 54 hours, about 60 hours, about 66 hours, about 72 hours, about 78 hours, about 84 hours, about 90 hours, or about 96 hours.

In some embodiments, the first culturing step is performed for a period of 42-56 hours, such as about 48 hours. In some embodiments, the second culturing step is performed for a period of 42-56 hours, such as about 48 hours.

In some embodiments, the first culturing step is performed for a period of 42-96 hours, such as about 72 hours. In some embodiments, the second culturing step is performed for a period of 42-56 hours, such as about 48 hours.

In some embodiments, all or a part of the first and/or second culturing step is performed under hypoxic conditions. In some embodiments, all or a part of the second culturing step is performed under hypoxic conditions. In some embodiments, the last 6-72 hours, the last 10-48 hours, or the last 12-36 hours, of the second culturing step is performed under hypoxic conditions. In some embodiments, the hypoxic condition is an 02 concentration that is between 0% and 15%, between 0% and 10%, or less than 10%, less than 9%, less than 8%, less than 7%, less than 6%, less than 5%, less than 4%, less than 3%, less than 2%, or less than 1%.

In some embodiments, all or a part of the first and/or second culturing step is performed under normoxic conditions. In some embodiments, all or a part of the second culturing step is performed under normoxic conditions. In some embodiments, at least the last 6-72 hours, the last 10-48 hours, or the last 12-36 hours, of the second culturing step is performed under normoxic conditions. In some embodiments, the normoxic condition is an 02 concentration that is between 20% and 21%.

In some embodiments, all or a part of the first and/or second culturing step is performed in the presence of insulin. In some embodiments, all or a part of the first culturing step is performed in the presence of insulin. In some embodiments, the first culturing step comprises culturing in the presence of insulin for at least 24 hours, at least 48 hours, or at least 72 hours. In some embodiments, all or a part of the second culturing step is performed in the presence of insulin. In some embodiments, the second culturing step comprises culturing in the presence of insulin for at least 24 hours, at least 48 hours, or at least 72 hours.

In some embodiments, the one or more progenitor cells are washed, using one or more washing steps, between the first and second culturing steps. In some embodiments, the washing medium may comprise a liquid medium (e.g., alpha-MEM, DMEM) optionally containing one or more supplements. In some embodiments, the supplement is a carbon source (e.g., glucose). In some embodiments, the one or more progenitor cells are not washed between the first and second culturing steps (for instance, the first culture medium is removed and the second culture medium is then added).

The first and/or second culturing steps can be performed in suspension, or attached to a solid support. The culturing may be two-dimensional or three-dimensional cell culturing.

For instance, in some embodiments, the culture vessel used for culturing may be a flask, flask for tissue culture (e.g., T25, T75), hyperflask (e.g., CellBind surface HYPERFlask®; Corning, Ref: 10024) or hyperstack (e.g., 12 or 36 chamber, HYPERStacks®, Corning, Refs: 10012, 10036, 10013, 10037), dish, petri dish, dish for tissue culture, multi dish, micro plate, micro-well plate, multi plate, multi-well plate, micro slide, chamber slide, tube, tray, CellSTACK® Chambers (e.g., 1ST, 2ST, 5ST, 10ST; Corning, Refs: 3268, 3269, 3313, 3319), culture bag, roller bottle, bioreactor, stirred culture vessel, spinner flask, microcarrier, or a vertical wheel bioreactor, for example. The one or more progenitor cells may be cultured in a volume of at least or about 0.2, 0.5, 1, 2, 5, 10, 15, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, 1 L, 5 L, 10 L, 50 L, 100 L, 1000 L, 5000 L, or 10,000 L, for example.

In embodiments in which culturing comprises two-dimensional cell culture, such as on the surface of a culture vessel, the culture surface (to which the cells are intended to adhere) may be coated with one or more substances that promote cell adhesion. Such substances useful for enhancing attachment to a solid support include, for example, type I, type II, and type IV collagen, concanavalin A, chondroitin sulfate, fibronectin, fibronectin-like polymers, gelatin, laminin, poly-D and poly-L-lysine, Matrigel, thrombospondin, osteopontin, poly-D-lysine, human extracellular matrix, Corning® Cell-Tak™ Cell and Tissue Adhesive, Corning PuraMatrix® Peptide Hydrogel, and/or vitronectin.

In some embodiments, where culturing of cells is performed as adherent culture, e.g., where cells are adhered to a solid support, cells may be seeded at an amount of 25,000-250,000 cells per cm²; 50,000-200,000 cells per cm²; 75,000-175,000 cells per cm²; or between 100,000-150,000 cells per cm².

In some embodiments, where culturing of cells is performed as adherent culture, e.g., where cells are adhered to a solid support, cells may be seeded to the solid support under gravitational force. In other embodiments, the cells may be seeded to the solid support under centrifugation.

In some embodiments, following the second culturing step, the second serum-free culture medium used in the second culturing step is recovered to obtain a conditioned medium containing the secretome of the one or more progenitor cells.

The recovered, conditioned medium may in some embodiments be subjected to one or more further processing steps. Following the second culturing step, the second serum-free culture medium used in the second culturing step may be removed, analyzed, recovered, concentrated, enriched, isolated, purified, refrigerated, frozen, cryopreserved, lyophilized, sterilized, etc.

In some embodiments, the recovered, conditioned medium may be pre-cleared or clarified to remove particulates of greater than a certain size. For instance, the recovered, conditioned medium may be pre-cleared or clarified by one or more centrifugation and/or filtration techniques.

In some embodiments, the recovered, conditioned medium is further processed to obtain a particular extract or fraction of the recovered, conditioned medium. For instance, the recovered, conditioned medium may be further processed to separate a small extracellular vesicle-enriched fraction (sEV) therefrom. An sEV fraction may be separated from the recovered, conditioned medium (or from a previously processed extract or fraction thereof) by one or more techniques such as centrifugation, ultracentrifugation, filtration, ultrafiltration, gravity, sonication, density-gradient ultracentrifugation, tangential flow filtration, size-exclusion chromatography, ion-exchange chromatography, affinity capture, polymer-based precipitation, or organic solvent precipitation, for example.

In some embodiments, conditioned medium is subjected to clarification by one or more filtration steps. In some embodiments thereof, one or more of the filtration steps utilizes a filter membrane having a particular pore size. In some embodiments thereof, a filter is used having a pore size of between 0.1 μm and 500 μm, or between 0.2 μm and 200 μm; or having a pore size less than or equal to 500 μm, 400 μm, 300 μm, 200 μm, 100 μm, 50 μm, 40 μm, 30 μm, 20 μm, 15 μm, 10 μm, 5 μm, 4 μm, 3 μm, 2 μm, 1 μm, 0.9 μm, 0.8 μm, 0.7 μm, 0.6 μm, 0.5 μm, 0.4 μm, 0.3 μm, 0.2 μm or 0.1 μm.

In some embodiments, the clarification comprises at least 1, at least 2, at least 3, at least 4, at least 5, at least 6, or at least 7, filtration steps. In some embodiments, the clarification comprises 4 filtration steps. In some embodiments, successive filtration steps utilize filters having increasingly smaller pores.

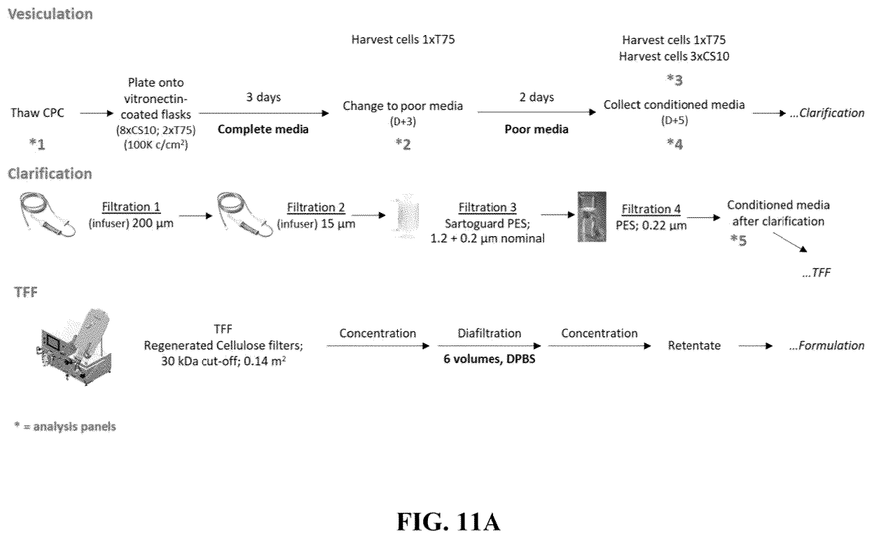

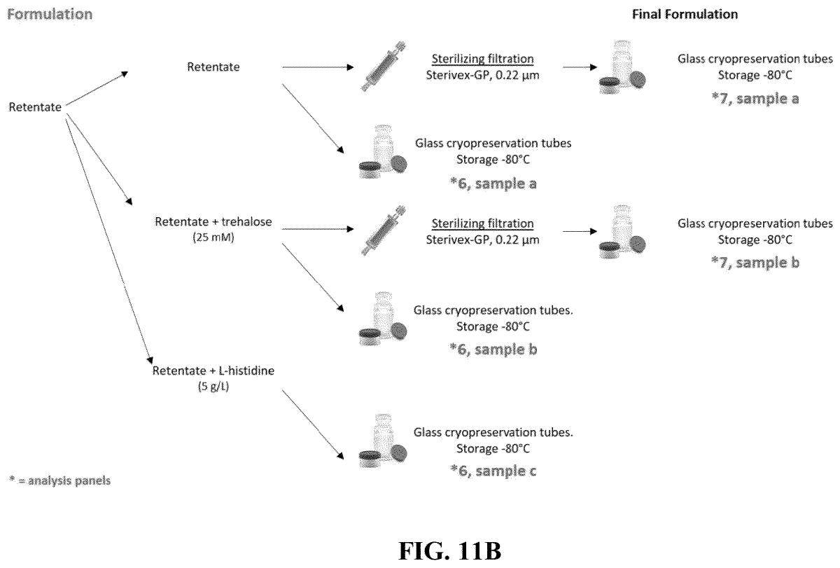

In some embodiments thereof, a first filtration step comprises use of an approximately 200 μm filter (e.g., a 200 μm drip chamber filter; Gravity Blood set, BD careFusion, Ref: VH-22-EGA); a second filtration step comprises use of an approximately 15 μm filter (e.g., DIDACTIC, Ref: PER1FL25); a third filtration step comprises use of an approximately 0.2 μm filter, optionally containing a pre-filter, for example, an approximately 1.2 μm pre-filter (e.g., Sartoguard PES XLG MidiCaps, pore sizes: 1.2 μm+0.2 μm, Sartorius, Ref: 5475307F7-OO-A); and a fourth filtration step comprises use of an approximately 0.22 μm filter (e.g., Vacuum Filter/Storage Bottle System, 0.22 μm pore, 33.2 cm² PES Membrane, Corning, Ref: 431097), as illustrated in Example 5 and in FIG. 11A.

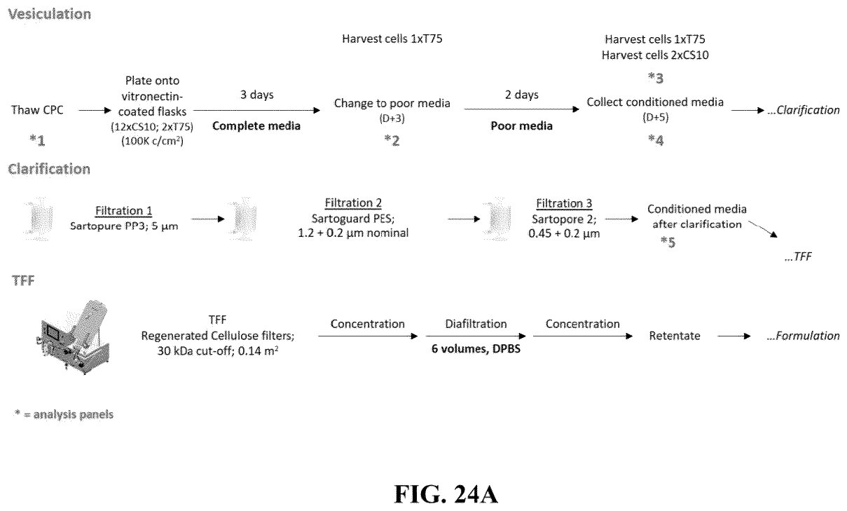

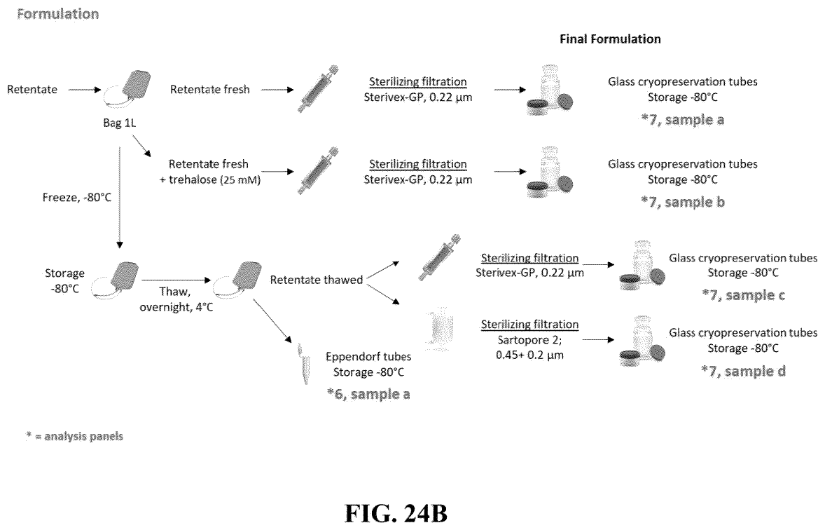

In other embodiments thereof, a first filtration step comprises use of an approximately 5 μm filter (e.g., Sartopure PP3 MidiCaps, pore size: 5 μm, Sartorius, Ref: 5055342P9-OO-A); a second filtration step comprises use of an approximately 0.2 μm filter, optionally containing a pre-filter, for example, an approximately 1.2 μm pre-filter (e.g., Sartoguard PES MidiCaps, pore sizes: 1.2 μm+0.2 μm, Sartorius, Ref: 5475307F9-OO-A, and a third filtration step comprises use of an approximately 0.2 μm filter, optionally containing a pre-filter, for example, an approximately 0.45 μm pre-filter (e.g., Sartopure 2 MidiCaps, pore sizes: 0.45 μm+0.2 μm, Sartorius, Ref: 5445307H8-OO-A), as illustrated in Example 12 and in FIG. 24A.

In some embodiments, conditioned medium may be subjected to clarification by one or more centrifugation steps. In some embodiments, conditioned medium may be subjected to clarification by a combination of centrifugation and filtration step(s).

In some embodiments, one or more additives are added to the conditioned medium, such as before clarification, and/or after clarification. In some embodiments, an additive is added that reduces aggregation. In some embodiments thereof, the additive is one or more selected from trehalose, histidine (e.g., L-histidine), arginine (e.g., L-arginine), citrate-dextrose solution, a Dnase (e.g., Dnase I), ferric citrate, or Anti-Clumping Agent (Gibco/Life technologies, Ref: 01-0057; Lonza, Ref: BE02-058E).

In some embodiments, conditioned medium or sEV may be subjected to isolation, enrichment, and/or concentration step(s) using tangential flow filtration (TFF). In some embodiments, the conditioned medium or sEV is subjected to TFF after clarification that employed one or more clarification steps (e.g., such as after one or more filtration and/or centrifugation steps). TFF is a rapid and efficient method for separating, enriching and purifying biomolecules. In some embodiments, TFF can be used, e.g., for concentrating (e.g., concentrating small extracellular vesicles from conditioned media); for diafiltration; and for concentrating and diafiltration. Diafiltration is a type of ultrafiltration process in which the retentate (the fraction that does not pass through the membrane) is diluted with buffer and re-ultrafiltered, to reduce the concentration of soluble permeate components and increase further the concentration of retained components.

In some embodiments, TFF is used for enriching, concentrating and diafiltration of conditioned medium or sEV (e.g., for concentration and diafiltration of EV secretome). In some embodiments, TFF is first used to concentrate conditioned medium or sEV, and is subsequently used for diafiltration. In some embodiments, a TFF process may comprise a further step of concentrating after diafiltration. In some embodiments, TFF is used for diafiltration but not concentrating. In some embodiments, TFF is used for concentrating but not diafiltration.

In some embodiments, the TFF membrane has a cut-off value of or less than 10 kDa, of or less than 20 kDa, of or less than 30 kDa, of or less than 40 kDa, of or less than 50 kDa, of or less than 60 kDa, of or less than 70 kDa, of or less than 80 kDa, of or less than 90 kDa, of or less than 100 kDa, or of or less than 150 kDa. In some embodiments, the TFF membrane has a cut-off value of about 10 kDa, about 30 kDa, about 100 kDa, or about 500 kDa. In some embodiments, the TFF membrane has a cut-off value of 30 kDa or about 30 kDa.

In some embodiments, the TFF membrane comprises cellulose. In some embodiments, the TFF membrane comprises regenerated cellulose. In some embodiments, the TFF membrane comprises a polyethersulfone (PES) membrane.

In some embodiments, conditioned media or sEV subjected to TFF can be further purified, isolated, and/or enriched (after TFF) using one or more purification, isolation, and/or enrichment, techniques. For instance, the resulting product from TFF can be subjected to a chromatography step, such as an ion exchange chromatography step or a steric exclusion chromatography step, to even further purify small extracellular vesicles. In some embodiments, conditioned media subjected to TFF, with or without further purification, isolation, and/or enrichment, may be further concentrated, such as by ultracentrifugation.

Any of the above-described processing techniques can be performed on recovered, conditioned medium (or a previously processed extract or fraction thereof) that is fresh, or has previously been frozen and/or refrigerated, for example.

In some embodiments, secretome-, extracellular vesicle-, and sEV-containing compositions produced by the methods herein may have added thereto at least one additive to prevent aggregation. The additive may be one or more selected from trehalose, histidine (e.g., L-histidine), arginine (e.g., L-arginine), citrate-dextrose solution, a Dnase (e.g., Dnase I), ferric citrate, or Anti-Clumping Agent (Gibco/Life technologies, Ref: 01-0057; Lonza, Ref: BE02-058E). In some embodiments, trehalose is added. In some embodiments, trehalose or L-histidine is added.

In some embodiments, the sEV fraction is CD63⁺, CD81⁺, and/or CD9⁺. The sEV fraction may contain one or more extracellular vesicle types, such as, for example, one or more of exosomes, microparticles, and extracellular vesicles. The sEV fraction may also contain secreted proteins (enveloped and/or unenveloped). Extracellular vesicles within conditioned media or sEV fractions of the present disclosure may contain, for example, one or more components selected from tetraspanins (e.g., CD9, CD63 and CD81), ceramide, MHC class I, MHC class II, integrins, adhesion molecules, phosphatidylserine, sphingomyelin, cholesterol, cytoskeletal proteins (e.g., actin, gelsolin, myosin, tubulin), enzymes (e.g., catalase, GAPDH, nitric oxide synthase, LT synthases), nucleic acids (e.g., RNA, miRNA), heat shock proteins (e.g., HSP70 and HSP90), exosome biogenesis proteins (ALIX, Tsg101), LT, prostaglandins, and S100 proteins.

In some embodiments, the presence of desired extracellular vesicle types in a fraction can be determined, for example, by nanoparticle tracking analysis (to determine the sizes of particles in the fraction); and/or by confirming the presence of one or more markers associated with a desired extracellular vesicle types. For instance, a fraction of recovered, conditioned media can be analyzed for the presence of desired extracellular vesicle types by detecting the presence of one or more markers in the fraction, such as, for example, CD9, CD63 and/or CD81.

In some embodiments, an sEV formulation or composition is positive for CD9, CD63 and CD81 (canonical EV markers), and is positive for the cardiac-related markers CD49e, ROR1, SSEA-4, MSCP, CD146, CD41b, CD24, CD44, CD236, CD133/1, CD29 and CD142. In some embodiments, an sEV formulation or composition contains a lesser amount of one or more markers selected from the group consisting of CD3, CD4, CD8, HLA-DRDPDQ, CD56, CD105, CD2, CD1c, CD25, CD40, CD11c, CD86, CD31, CD20, CD19, CD209, HLA-ABC, CD62P, CD42a and CD69, as compared to the amount of CD9, CD63 and/or CD81 in the sEV formulation or composition. In some embodiments, an sEV formulation or composition contains an undetectable amount of (e.g., by MACSPlex assay, by immunoassay, etc.), or is negative for, one or more markers selected from the group consisting of CD19, CD209, HLA-ABC, CD62P, CD42a and CD69.

In some embodiments, the sEV formulation or composition is at least one of the following: an sEV formulation or composition that has been enriched for extracellular vesicles having a diameter of between about 50-200 nm or between 50-200 nm; an sEV formulation or composition that has been enriched for extracellular vesicles having a diameter of between about 50-150 nm or between 50-150 nm; an sEV formulation or composition that is substantially free or free of whole cells; and an sEV formulation or composition that is substantially free of one or more culture medium components (e.g., phenol-red).

In some embodiments, such as, for example, some GMP-compatible processes, testing panels are conducted to analyze and/or determine one or more properties of the processes, products thereof, or intermediate products, etc.

For instance, during the vesiculation stage (including, e.g., thawing, plating, culturing and/or harvesting steps), one or more properties of the cells may be examined (including, for example: the number of viable cells, the percentage viability of the cells; morphologies of the cells; identity of the cells; karyotype of the cells; and/or transcriptome of the cells).

Additionally, or alternatively, one or more properties of a secretome and/or extracellular vesicle-containing fraction, extract, or composition can be analyzed using one or more tests (including, e.g., particle concentration and/or particle size distribution; protein concentration; protein profile concentration; RNA profile; potency; marker identity; host cell protein assessment; residual DNA quantification and/or characterization; sterility; mycoplasma; endotoxin; appearance; pH; osmolarity; extractable volume; hemolytic activity; complement activation; platelet activation; and/or genotoxicity), to determine one or more properties of the secretome/extracellular vesicles. For instance, one or more of these properties can be assessed on conditioned media before clarification; on conditioned media after clarification; on isolated and/or concentrated secretome/extracellular vesicles; and/or on final formulations. In some embodiments, final formulations may be tested immediately after production and/or 1-week, 2-weeks, 1-month, 2-months, 3-months, 6-months, 1-year or several years, after being formulated.

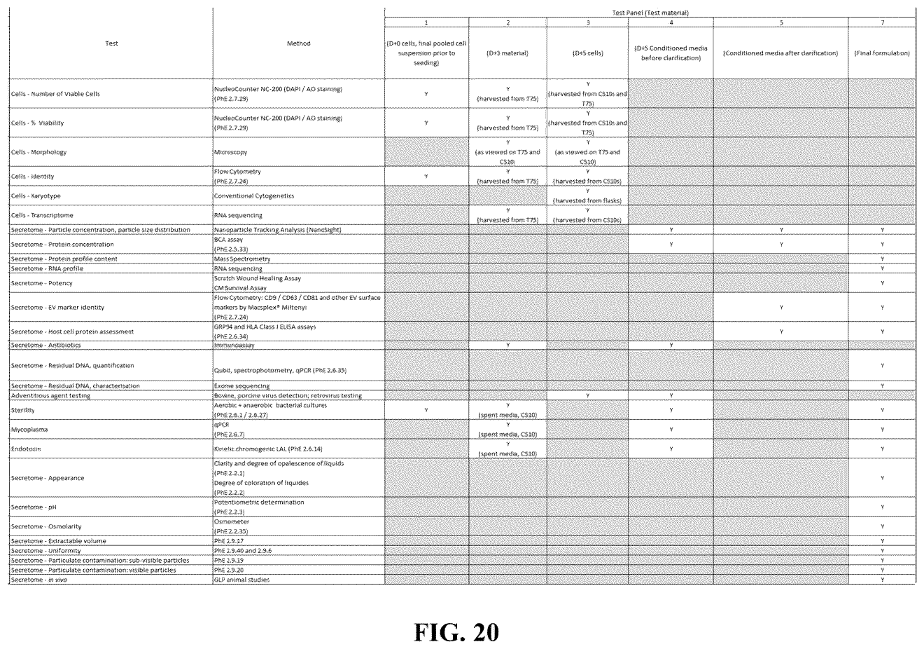

An exemplary process/product testing panel is shown in FIG. 20.

Therapeutic Compositions and Applications

The present disclosure contemplates the generation of secretome-, extracellular vesicle-, and sEV-containing compositions useful as therapeutic agents. In some embodiments, the methods of the present disclosure comprise administering an effective amount of a secretome-, extracellular vesicle-, and/or sEV-containing composition to a subject in need thereof.

Tissues treated according to the methods of the present disclosure include, without limitation, cardiac tissue, brain or other neural tissue, skeletal muscle tissue, pulmonary tissue, arterial tissue, capillary tissue, renal tissue, hepatic tissue, tissue of the gastrointestinal tract, epithelial tissue, connective tissue, tissue of the urinary tract, etc. The tissue to be treated may be damaged or fully or partly non-functional due to an injury, age-related degeneration, acute or chronic disease, cancer, or infection, for example. Such tissues may be treated, for example, by intravenous administration of a secretome-, extracellular vesicle-, and/or sEV-containing composition.

In some embodiments, compositions of the present disclosure may be used to treat diseases such as myocardial infarction, stroke, heart failure, and critical limb ischemia, for example. In some embodiments, compositions of the present disclosure may be used to treat heart failure which has one or more of the following characteristics: is acute, chronic, ischemic, non-ischemic, with ventricular dilation, without ventricular dilation, with reduced left ventricular ejection fraction, or with preserved left ventricular ejection fraction. In some embodiments, compositions of the present disclosure may be used to treat heart failure selected from the group consisting of ischemic heart disease, cardiomyopathy, myocarditis, hypertrophic cardiomyopathy, diastolic hypertrophic cardiomyopathy, dilated cardiomyopathy, and post-chemotherapy induced heart failure. In some embodiments, compositions of the present disclosure may be used to treat diseases such as congestive heart failure, heart disease, ischemic heart disease, valvular heart disease, connective tissue diseases, viral or bacterial infection, myopathy, dystrophinopathy, liver disease, renal disease, sickle cell disease, diabetes, ocular diseases, and neurological diseases. It will be recognized that a suitable progenitor cell type(s) may be selected depending on the disease to be treated, or the tissue to be targeted.

For example, in some embodiments, a subject with a cardiac disease, such as acute myocardial infarction or heart failure, can be treated with a secretome-, extracellular vesicle-, and/or sEV-containing composition, produced from cardiomyocyte progenitor cells, cardiac progenitor cells, and/or cardiovascular progenitor cells.

Additionally, a secretome-, extracellular vesicle-, and/or sEV-containing composition produced from an appropriate progenitor cell type can also be used to improve the functioning or performance of a tissue. For instance, an improvement in angiogenesis, or an improvement in cardiac performance, may be effected by delivering a secretome-, extracellular vesicle-, and/or sEV-containing composition, produced from cardiomyocyte progenitor cells, cardiac progenitor cells, and/or cardiovascular progenitor cells, to a subject in need thereof.

In some embodiments, the administration comprises administration at a tissue or organ site that is the same as the target tissue. In some embodiments, the administration comprises administration at a tissue or organ site that is different from the target tissue. Such administration may include, for example, intravenous administration.

A secretome-, extracellular vesicle-, and/or sEV-containing composition may contain, or be administered with, a pharmaceutically-acceptable diluent, carrier, or excipient. Such a composition may also contain, in some embodiments, pharmaceutically acceptable concentrations of one or more of a salt, buffering agent, preservative, or other therapeutic agent. Some examples of materials which can serve as pharmaceutically acceptable carriers include sugars, such as lactose, glucose and sucrose; glycols, such as propylene glycol; polyols, such as glycerin, sorbitol, mannitol and polyethylene glycol; esters, such as ethyl oleate and ethyl laurate; buffering agents, such as magnesium hydroxide and aluminum hydroxide; pyrogen-free water; isotonic saline; Ringer's solution; ethyl alcohol; phosphate buffer solutions; and other nontoxic compatible substances employed in pharmaceutical formulations. For instance, in some embodiments, a secretome-, extracellular vesicle-, and/or sEV-containing composition, may be formulated with a biomaterial, such as an injectable biomaterial. Exemplary injectable biomaterials are described, for example, in WO 2018/046870, incorporated by reference herein in its entirety.

The secretome-, extracellular vesicle-, and/or sEV-containing compositions of the present disclosure may be administered in effective amounts, such as therapeutically effective amounts, depending on the purpose. An effective amount will depend upon a variety of factors, including the material selected for administration, whether the administration is in single or multiple doses, and individual patient parameters including age, physical condition, size, weight, and the stage of disease. These factors are well known to those of ordinary skill in the art.

Any appropriate route of administration may be employed, for example, administration may be parenteral, intravenous, intra-arterial, subcutaneous, intratumoral, intramuscular, intracranial, intraorbital, ophthalmic, intraventricular, intrahepatic, intracapsular, intrathecal, intracisternal, intraperitoneal, intranasal, intramyocardial, intra-coronary, aerosol, suppository, epicardial patch, oral administration, or by perfusion. For instance, therapeutic compositions for parenteral administration may be in the form of liquid solutions or suspensions; for oral administration, formulations may be in the form of tablets or capsules; and for intranasal formulations, in the form of powders, nasal drops, or aerosols. For instance, in some embodiments, a subject with a cardiac disease, such as acute myocardial infarction or heart failure, can be treated with a secretome-, extracellular vesicle-, and/or sEV-containing composition, produced from cardiomyocyte progenitor cells, cardiac progenitor cells, and/or cardiovascular progenitor cells, wherein the composition is administered intravenously.

In some embodiments, a single dose of a secretome-, extracellular vesicle-, and/or sEV-containing composition may be administered. In other embodiments, multiple doses, spanning one or more doses per day, week, or month, are administered to the subject. In some embodiments, single or repeated administration of a secretome-, extracellular vesicle-, and/or sEV-containing composition, including two, three, four, five or more administrations, may be made. In some embodiments, the secretome-, extracellular vesicle-, and/or sEV-containing composition may be administered continuously. Repeated or continuous administration may occur over a period of several hours (e.g., 1-2, 1-3, 1-6, 1-12, 1-18, or 1-24 hours), several days (e.g., 1-2, 1-3, 1-4, 1-5, 1-6 days, or 1-7 days) or several weeks (e.g., 1-2 weeks, 1-3 weeks, or 1-4 weeks), depending on the nature and/or severity of the condition being treated. If administration is repeated but not continuous, the time in between administrations may be hours (e.g., 4 hours, 6 hours, or 12 hours), days (e.g., 1 day, 2 days, 3 days, 4 days, 5 days, or 6 days), or weeks (e.g., 1 week, 2 weeks, 3 weeks, or 4 weeks). The time between administrations may be the same or they may differ. As an example, if symptoms worsen, or do not improve, the secretome-, extracellular vesicle-, and/or sEV-containing composition, may be administered more frequently. Contrarily, if symptoms stabilize or diminish, the secretome-, extracellular vesicle-, and/or sEV-containing composition may be administered less frequently.

In some embodiments, a secretome-, extracellular vesicle-, and/or sEV-containing composition is administered in several doses, for example three, on or about several days, weeks, or months apart, for example two weeks apart, by intravenous administration. In some embodiments thereof, the composition may be diluted with, formulated with, and/or administered together with, a carrier, diluent, or suitable material (e.g., saline).

Assays for Determining Secretome and Extracellular Vesicle Activity, Functionality, and/or Potency

The present disclosure also encompasses methods for analyzing the activity, functionality, and/or potency, of conditioned media; or of a secretome-, extracellular vesicle-, and/or sEV-containing composition.

The activity, functionality, and/or potency, of conditioned media; or of a secretome-, extracellular vesicle-, and/or sEV-containing composition, can be assessed by various techniques, depending on, for example, the type of progenitor cells used to produce the conditioned media or composition, and the desired use of the conditioned media or composition.

For instance, the activity, functionality, and/or potency, of conditioned media; or of a secretome-, extracellular vesicle-, and/or sEV-containing composition, can be assessed by administering the conditioned media, secretome-, extracellular vesicle-, and/or sEV-containing composition, to target cells in vitro, ex vivo, or in vivo. One or more properties of the target cells can then be analyzed, such as, for example, cell viability, hypertrophy, cell health, cell adhesion, cell physiology, ATP content, cell number, and cell morphology, to determine the activity, functionality, and/or potency, of conditioned media; or of a secretome-, extracellular vesicle-, and/or sEV-containing composition.

In some embodiments, assays known in the art may be used to determine the activity, functionality, and/or potency, of conditioned media; or of a secretome-, extracellular vesicle-, and/or sEV-containing composition.

For instance, for conditioned media; or for a secretome-, extracellular vesicle-, and/or sEV-containing composition, obtained from cardiovascular progenitor cells or cardiomyocyte progenitor cells, the activity, functionality, and/or potency, thereof may be measured using a known cardiomyocyte viability assay, such as described in El Harane et al. (Eur. Heart J., 2018, 39(20): 1835-1847).

Specifically, serum-deprived cardiac myoblasts (e.g., H9c2 cells) may be contacted with conditioned media; or a secretome-, extracellular vesicle-, and/or sEV-containing composition, and the viability of the cells measured thereafter. In some embodiments of this assay, the cells are deprived of serum before administering the conditioned media or the secretome-, extracellular vesicle-, and/or sEV-containing composition. In other embodiments, the cells are deprived of serum after administering the conditioned media or the secretome-, extracellular vesicle-, and/or sEV-containing composition. In some embodiments, the cells are deprived of serum before and after administering the conditioned media or the secretome-, extracellular vesicle-, and/or sEV-containing composition.

In other embodiments, the angiogenic activity of a conditioned media or a secretome-, extracellular vesicle-, and/or sEV-containing composition, can be measured, for example, using a HUVEC scratch wound healing assay. In HUVEC scratch wound healing assays, HUVEC cells are cultured on a culture surface, and the cultured cell layer(s) is then scratched; angiogenic activity of a conditioned media or a secretome-, extracellular vesicle-, and/or sEV-containing composition, can then be determined by the capacity of the conditioned media or the secretome-, extracellular vesicle-, and/or sEV-containing composition, to produce closure of the wound under serum-free conditions.

Cell viability (in cell viability assays) may be measured using, for example, a DNA-labeling dye or a nuclear-staining dye. The dye may be used with live cell imaging.

An activity, functionality, and/or potency, of conditioned media; or of a secretome-, extracellular vesicle-, and/or sEV-containing composition, may also be determined with reference to one or more control samples. For instance, control cells may be one or more of: serum-deprived control cells which are not administered the conditioned media or the secretome-, extracellular vesicle-, and/or sEV-containing composition; control cells which are not serum-deprived; or serum-deprived control cells which are administered a mock conditioned media or mock secretome-, extracellular vesicle-, and/or sEV-containing composition.

In some methods of the present disclosure, an activity, functionality, and/or potency, of conditioned media; or of a secretome-, extracellular vesicle-, and/or sEV-containing composition, can be assessed by a method comprising administering the conditioned media or the secretome-, extracellular vesicle-, and/or sEV-containing composition, to target cells cultured under at least one stress-inducing condition, and analyzing at least one property of the cells. The one or more properties of the target cells that may be analyzed can be selected from, for instance, cell migration, cell survival, cell viability, hypertrophy, cell health, cell adhesion, cell physiology, ATP content, cell number, and cell morphology. In some embodiments, the at least one property measured is cell adhesion, cell number, cell growth, and/or cell morphology, and wherein the cell adhesion, cell number, cell growth, and/or cell morphology, is determined by measuring electrical impedance across a culture vessel surface in the culture.

In a first method thereof, target cells are cultured in a pre-treatment medium under at least one stress-inducing condition, followed by administering a conditioned medium or a secretome-, extracellular vesicle-, and/or sEV-containing composition, to the cell culture. The target cells are then cultured in the presence of the conditioned medium or the secretome-, extracellular vesicle-, and/or sEV-containing composition, and at least one property of the cultured cells is measured one or more times during the culturing. In some embodiments, the at least one property is measured multiple times during the culturing in the presence of the conditioned medium or the secretome-, extracellular vesicle-, and/or sEV-containing composition (such as, for example, 5 minutes to 10 hours apart from each other; 10 minutes to 4 hours apart from each other; or 30 minutes to 2 hours apart from each other).

In some embodiments of this first method, the culturing in the presence of the conditioned medium or the secretome-, extracellular vesicle-, and/or sEV-containing composition, occurs in the presence of the at least one stress-inducing condition. In other embodiments of this first method, the culturing in the presence of the conditioned medium or the secretome-, extracellular vesicle-, and/or sEV-containing composition, occurs in the absence of the at least one stress-inducing condition.

In some embodiments of this first method, the pre-treatment medium is removed from the cells before the culturing in the presence of the conditioned medium or the secretome-, extracellular vesicle-, and/or sEV-containing composition. Thus, in embodiments of the first method where the at least one stress-inducing condition is provided by the pre-treatment medium (e.g., by a stress-inducing agent present in the pre-treatment medium), the culturing in the presence of the conditioned medium or the secretome-, extracellular vesicle-, and/or sEV-containing composition, occurs in the absence of the at least one stress-inducing condition.

In other embodiments of this first method, the pre-treatment medium is not removed from the cells before the culturing in the presence of the conditioned medium or the secretome-, extracellular vesicle-, and/or sEV-containing composition. Thus, in embodiments of the first method where the at least one stress-inducing condition is provided by the pre-treatment medium (e.g., by a stress-inducing agent present in the pre-treatment medium), the culturing in the presence of the conditioned medium or the secretome-, extracellular vesicle-, and/or sEV-containing composition, occurs in the presence of the at least one stress-inducing condition.

In a second method, target cells are cultured in a pre-treatment medium, followed by administering a conditioned medium or a secretome-, extracellular vesicle-, and/or sEV-containing composition (and optionally thereafter, culturing the target cells in the presence of the conditioned medium or the secretome-, extracellular vesicle-, and/or sEV-containing composition). The target cells are then cultured under at least one stress-inducing condition, and at least one property of the cultured cells is measured one or more times during the culturing under the at least one stress-inducing condition (which also occurs in the presence of the conditioned medium or the secretome-, extracellular vesicle-, and/or sEV-containing composition). In some embodiments, the at least one property is measured multiple times during the culturing under the at least one stress-inducing condition (and in the presence of the conditioned medium or the secretome-, extracellular vesicle-, and/or sEV-containing composition), such as, for example, 5 minutes to 10 hours apart from each other; 10 minutes to 4 hours apart from each other; or 30 minutes to 2 hours apart from each other.

In some embodiments of this second method, the target cells are cultured in the presence of the conditioned medium or the secretome-, extracellular vesicle-, and/or sEV-containing composition, before being cultured under the at least one stress-inducing condition. In other embodiments of this second method, the target cells are not cultured in the presence of the conditioned medium or the secretome-, extracellular vesicle-, and/or sEV-containing composition, before being cultured under the at least one stress-inducing condition. In some embodiments of this second method, the conditioned medium or the secretome-, extracellular vesicle-, and/or sEV-containing composition, is removed from the target cells before the target cells are cultured in the presence of the at least one stress-inducing condition.

In some embodiments of the above first and second methods, the stress-inducing condition is culturing in the presence of a cellular stress agent. In some embodiments of the second method, the cellular stress agent is co-administered to the target cells with the conditioned medium or the secretome-, extracellular vesicle-, and/or sEV-containing composition.

In some embodiments of the above first and second methods, the cellular stress agent is one or more apoptosis-inducing agents.

The one or more apoptosis-inducing agents may be selected from, for example, doxorubicin, staurosporine, etoposide, camptothecin, paclitaxel, vinblastine, gambogic acid, daunorubicin, tyrphostins, thapsigargin, okadaic acid, mifepristone, colchicine, ionomycin, 24(S)-hydroxycholesterol, cytochalasin D, brefeldin A, raptinal, carboplatin, C2 ceramide, actinomycin D, rosiglitazone, kaempferol, berberine chloride, bioymifi, betulinic acid, tamoxifen, embelin, phytosphingosine, mitomycin C, birinapant, anisomycin, genistein, cycloheximide, and the like.

In some embodiments, the apoptosis-inducing agent is an indolocarbazole. In some embodiments, the apoptosis-inducing agent is an indolo(2,3-a)pyrrole(3,4-c)carbazole. In some embodiments, the apoptosis-inducing agent is staurosporine, or a derivative thereof. In other embodiments, the apoptosis-inducing agent is doxorubicin, or a derivative thereof.

In some embodiments of the first and second methods, the at least one property measured is viability of the cultured cells. The viability may be measured, for example, using a DNA-labeling dye or a nuclear-staining dye. In some embodiments thereof, the DNA-labeling dye or the nuclear-staining dye is a fluorescent dye, such as a far-red fluorescent dye.

In some embodiments of the first and second methods, one or more of the culturing of the target cells with: (a) the pre-treatment medium; (b) the conditioned medium or a secretome-, extracellular vesicle-, and/or sEV-containing composition; and (c) at least one stress-inducing condition, may occur in the absence of serum. In some embodiments, the target cells may be deprived of serum before administering the conditioned media or the secretome-, extracellular vesicle-, and/or sEV-containing composition. In other embodiments, the target cells may be deprived of serum after administering the conditioned media or the secretome-, extracellular vesicle-, and/or sEV-containing composition. In some embodiments, the cells are deprived of serum before and after administering the conditioned media or the secretome-, extracellular vesicle-, and/or sEV-containing composition.

In embodiments of the first and second methods, the target cells can be cultured in the pre-treatment medium for differing lengths of time. For instance, the target cells can be cultured in the pre-treatment medium for 30 minutes to 10 hours, 1 hour to 5 hours, or more than, less than, or about, 1 hour, 2 hours, 3 hours, 4 hours, or 5 hours.

In embodiments of the first and second methods, the target cells are cultured with the conditioned medium or the secretome-, extracellular vesicle-, and/or sEV-containing composition, for at least 30 minutes, at least 1 hour, at least 2 hours, at least 4 hours, at least 6 hours, at least 8 hours, at least 12 hours, at least 18 hours, at least 24 hours, at least 36 hours, or at least 48 hours.

In some embodiments of the first and second methods, the target cells are cultured in vitro prior to culturing in the pre-treatment medium. For instance, the target cells may be cultured in vitro for between 1-21 days, between 3-17 days, between 5-14 days, or less than 20 days, less than 18 days, less than 16 days, less than 14 days, less than 12 days, less than 10 days, less than 8 days, less than 6 days, less than 4 days, or less than 2 days, prior to culturing in the pre-treatment medium. In certain embodiments in which the target cells are cultured in vitro prior to culturing in the pre-treatment medium, the target cells are supplied with fresh culture medium prior to culturing in the pre-treatment medium. For instance, the target cells may be supplied with fresh culture medium 6-72 hours, 8-60 hours, 10-48 hours, 12-36 hours, prior to culturing in the pre-treatment medium.

In embodiments of the first and second methods, the culturing of the target cells may be two-dimensional or three-dimensional cell culturing. For instance, in some embodiments, the culture vessel used for culturing may be a flask, flask for tissue culture, hyperflask, dish, petri dish, dish for tissue culture, multi dish, micro plate, micro-well plate, multi plate, multi-well plate, micro slide, chamber slide, tube, tray, CellSTACK® Chambers, culture bag, roller bottle, bioreactor, stirred culture vessel, spinner flask, microcarrier, or a vertical wheel bioreactor, for example.

In embodiments in which culturing comprises two-dimensional cell culture, such as on the surface of a culture vessel, the culture surface (to which the cells are intended to adhere) may be coated with one or more substances that promote cell adhesion. Such substances useful for enhancing attachment to a solid support include, for example, type I, type II, and type IV collagen, concanavalin A, chondroitin sulfate, fibronectin, fibronectin-like polymers, gelatin, laminin, poly-D and poly-L-lysine, Matrigel, thrombospondin, and/or vitronectin.

In embodiments of the first and second methods, the at least one property may also be analyzed with reference to one or more control samples.

For instance, the first and second methods may further comprise culturing positive control cells in parallel, wherein the positive control cells are not administered the conditioned medium or the secretome-, extracellular vesicle-, and/or sEV-containing composition, and are not cultured under the at least one stress-inducing condition. Thus, in embodiments in which the stress inducing condition is the presence of an apoptosis-inducing agent, the positive control cells are not administered the apoptosis-inducing agent.

The first and second methods may comprise culturing negative control cells in parallel, wherein the negative control cells are not administered the conditioned medium or the secretome-, extracellular vesicle-, and/or sEV-containing composition. In some embodiments, the negative control cells comprise negative control cells subjected to the same steps as the target cells, except that they are not administered the secretome.

In certain embodiments, the negative control cells comprise negative control cells cultured in the pre-treatment medium under the at least one stress-inducing condition. The at least one property measured in the target cells may also then be measured in the negative control cells, either during or after they are cultured in the pre-treatment medium under the at least one stress-inducing condition.

In some embodiments, the negative control cells comprise negative control cells to which a mock conditioned medium or a mock secretome-, extracellular vesicle-, and/or sEV-containing composition is added. In specific embodiments thereof, the mock conditioned medium or the mock secretome-, extracellular vesicle-, and/or sEV-containing composition is produced by omitting cells from the process of producing a conditioned medium or a secretome-, extracellular vesicle-, and/or sEV-containing composition, such as a process of the present disclosure.

The use of such a negative control(s) allows an activity, functionality and/or potency, of a conditioned medium or a secretome-, extracellular vesicle-, and/or sEV-containing composition, to be evaluated. For instance, where the at least one property measured is viability of the cultured cells, a conditioned medium or a secretome-, extracellular vesicle-, and/or sEV-containing composition, may be determined to have an activity, functionality, potency (and/or exhibit a therapeutic effect), when the viability of the target cells is higher than the viability of the negative control cells.

Alternatively, for instance, where the at least one property measured is cell adhesion, cell growth, and/or cell number, and wherein the cell adhesion, cell growth, and/or cell number is determined by measuring electrical impedance across a culture vessel surface in the culture, a conditioned medium or a secretome-, extracellular vesicle-, and/or sEV-containing composition may be determined to have an activity, functionality, potency (and/or exhibit a therapeutic effect), when the electrical impedance across a culture vessel surface in the culture is higher than the electrical impedance across a culture vessel surface in a culture of negative control cells.

Any one or more samples, and/or any one or more positive and/or negative controls, may be performed in replicate, such as, for example, in duplicate, in triplicate, etc. In some embodiments thereof in which cell viability is measured, and where replicate cultures are performed, the number of positive control cells in the replicate cultures may be averaged to produce an average maximum cell number (and the number of target cells in each replicate test culture may be normalized to the average maximum cell number, to calculate cell viability).

To more accurately compare an activity, functionality, and/or potency, between different conditioned media or secretome-, extracellular vesicle-, and/or sEV-containing compositions, it may be beneficial to determine the amount of the conditioned medium or the secretome-, extracellular vesicle-, and/or sEV-containing composition, added to target cells. This can be determined, for example, based on one or more of: the amount of secreting cells that produced the secretome; the protein content of said secretome; the RNA content of said secretome; the exosome amount of said secretome; and particle number.

### EXPERIMENTAL

Non-limiting embodiments of the present invention are illustrated in the following Examples. Efforts have been made to ensure accuracy with respect to numbers used (e.g., amounts, concentrations, percent changes, and the like), but some experimental errors and deviations should be accounted for. It should be understood that these Examples are given by way of illustration only and are not intended to limit the scope of what the inventor regards as various embodiments of the present invention. Not all of the following steps set forth in each Example are required nor must the order of the steps in each Example be as presented.

### Example 1

### Generation of Cardiovascular Progenitor Cells from iPSCs

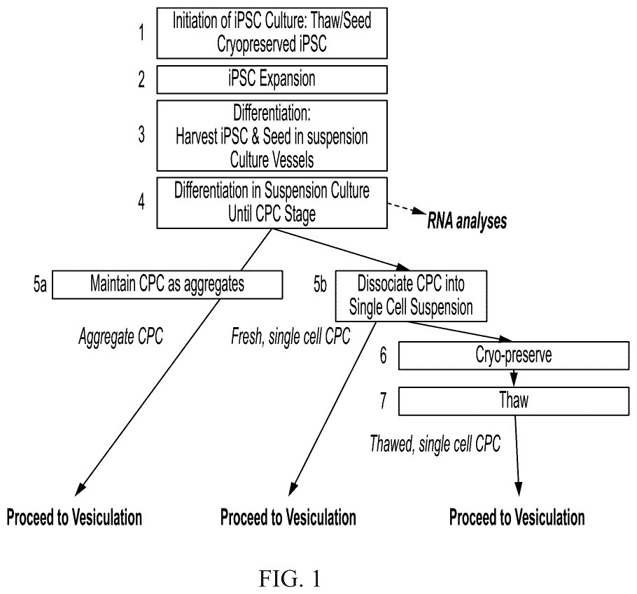

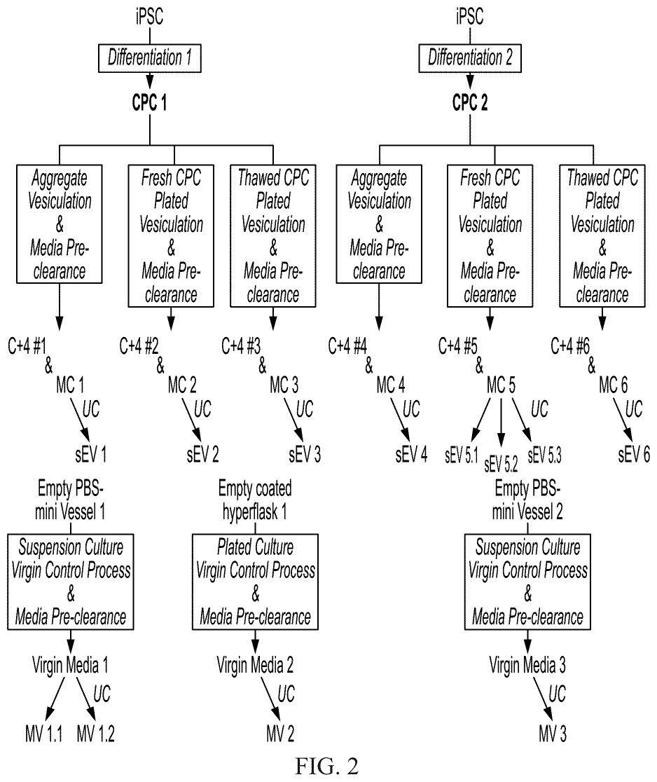

Human iPS cells (iPSCs) were expanded and differentiated into cardiovascular progenitor cells (CPCs) by suspension culture in PBS-mini vessels (PBS MINI 0.5 L Bioreactor Single Use Vessels; PBS Biotech ref: 1A-0.5-D-001), using the process depicted in FIG. 1. At the end of the CPC differentiation period, cells were counted as follows. A small sample (5-10 mL) of cell aggregates in suspension was removed from the suspension culture vessels, cell aggregates were gravity settled, supernatant removed and aggregates were resuspended in 3-5 mL of room temperature TrypLE Select (Invitrogen ref: 12563029), and incubated for 3-10 min at 37° C. Digestions were stopped using double the volume of RPMI-B27 Quench media (RPMI 1640 Medium (Gibco ref: 118875-085) supplemented with B-27 XenoFree, CTS grade 50×(Gibco ref: A14867-01, fc=1×), filter sterilized using a 0.2 μm filter (ThermoScientific ref 567-0020)). Cell suspensions were then centrifuged at 300×g for 5 minutes, and the resulting supernatant was discarded. The remaining cell pellets were delicately loosened, and the cells were resuspended in 5-10 mL of MEM alpha media base (MEM alpha, GlutaMAX™, no nucleosides, Gibco ref 32561-.37). Of these resuspended cells, one or two 500 μL samples were counted using a ViCell XR cell viability analyzer (Beckman Coulter), according to the manufacturer's directions. The viable cells per mL were noted. Two distinct differentiation runs were performed, as depicted in FIG. 2, and similar yields of CPC per input iPSC were obtained.

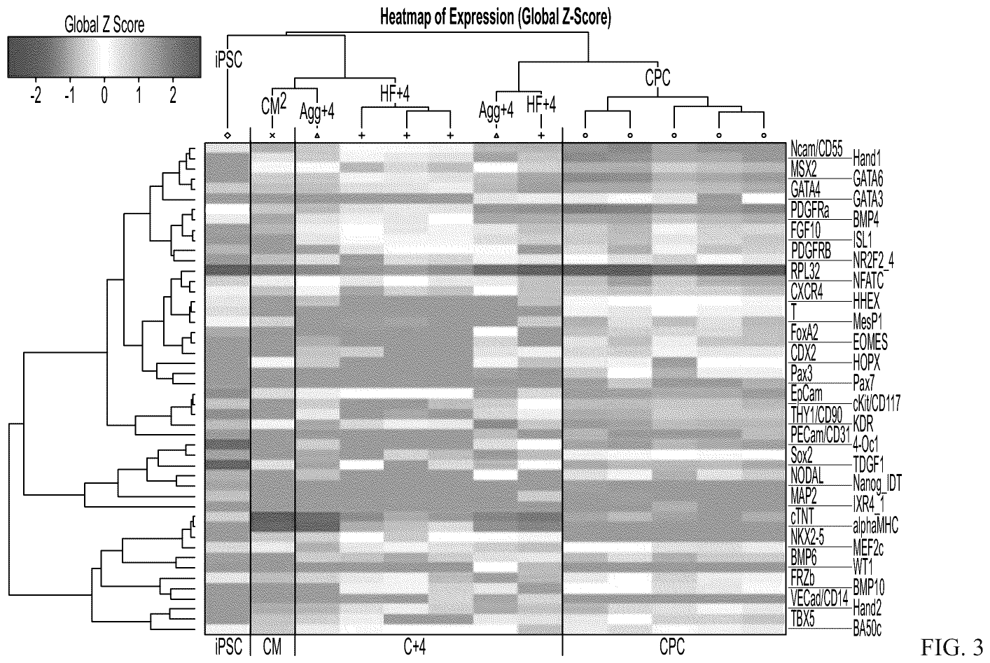

To confirm that the resulting cells were indeed CPCs, RNA expression by the resulting cells was analyzed. Specifically, between 1 and 2 million cells from the cell samples were removed and lysed in RLT plus buffer (Qiagen 1030963) for RNA extraction. RNA was extracted from the lysates using the Qiasymphony RNA kit (Qiagen, Ref: 931636), following the manufacturer's directions. mRNA levels for 48 custom selected genes were evaluated using the Fluidigm platform. Un-supervised hierarchical clustering was performed on raw data using the Fluidigm package. RNA expression by the resulting cells was compared to RNA expression by iPSC and cardiomyocyte control cells, confirming that gene expression by the resulting cells was consistent with them being CPCs (FIG. 3).

To dissociate CPC aggregates to single cells, 300-800 mL of the aggregate suspension of CPCs were collected from the differentiation suspension cultures, and allowed to settle for approximately 5 minutes in 500 mL conical tubes. Spent media was then removed, and cell aggregates were washed in DPBS−/−. The washed cell aggregates were then resuspended in room temperature TrypLE (in approximately 25 mL TrypLE for 100 mL original aggregate suspension volume) and were allowed to dissociate for 10 min at 37° C. The cell aggregate dissociations were quenched with an equal volume of RPMI-B27 Quench media, and the dissociated cells were spun at 400×g for 5 minutes. The resulting cell pellets were resuspended in RPMI-B27 Quench media, and then strained (Falcon 100 μm Cell strainer, Corning ref: 352360) into conical tubes and counted using a ViCell XR cell viability analyzer (Beckman Coulter).

A subset of these cells were re-spun at 300×g for 5 minutes, resuspended for fresh CPC plated vesiculation culture in alpha-MEM complete media (MEM alpha media base (MEM alpha, GlutaMAX™, no nucleosides, Gibco ref 32561-.37); Gentamicin (Gibco ref 15750060, final concentration (fc)=0.025 mg/mL); glucose supplement (Gibco ref A2494001, at a ratio of 1:200); Flexbumin (with 25% w/vol human serum albumin, Baxter ref: NDC0944-0493-02 code 2G0012, fc HSA=2 mg/mL); B27 (minus insulin) (50×, Gibco ref A1895601, fc=1×); Human FGF-2 Premium grade (Miltenyi Biotec ref: A12873-01, fc=1 ug/mL); filter sterilized using a 0.2 μm filter (ThermoScientific ref 567-0020); media were used the same day). The freshly harvested single cells were again counted using a ViCell XR cell viability analyzer (Beckman Coulter) and plated (see Example 2 below). The remainder of the single cell suspensions were spun at 400×g for 5 minutes, and the cells were resuspended in cryopreservation media (CryoStor CS-10, BioLife Solution ref: 210102) at 25 million cells/mL, frozen at −80° C., and then stored in liquid nitrogen for later use in thawed CPC plated vesiculation culture.

### Example 2

### Vesiculation Culture of Cardiovascular Progenitor Cells

CPCs were cultured in the vesiculation process as fresh aggregates in suspension culture, as fresh single cells plated onto hyperflasks, or as thawed single cells plated onto hyperflasks after having been cryopreserved and maintained at −80° C. or less until time of use. Specifically, CPCs produced in Example 1 were used in suspension vesiculation culture and in adherent vesiculation culture in hyperflasks as described below.

For suspension vesiculation culture, the volumes of aggregates in PBS-mini vessels at the end of the CPC differentiation process were noted (300-400 mL per vessel; “day+0” volumes). The cell aggregates underwent a 100% media exchange according to the following steps: (1) cell aggregates were transferred from PBS-mini vessels to conical tubes and allowed to settle for approximately 15 min; (2) PBS-mini vessels were rinsed three times with MEM alpha media base (MEM alpha, GlutaMAX™, no nucleosides, Gibco ref 32561-.37); (3) spent media was removed from settled cell aggregates; (4) cell aggregates were washed three times with an appropriate volume of MEM alpha media base; and (5) washed cell aggregates were re-seeded into their original (washed) PBS-mini vessels in alpha-MEM complete media (as described above) at their day+0 volumes to maintain cell density.

The seeded cell aggregates were then cultured in suspension (37° C., 5% CO₂, at atmospheric oxygen) with agitation at 40 rpm for 2 days (until “day+2”). At day+2, cell aggregates underwent a 100% media exchange following three rinses in MEM alpha media base. For this day+2 media exchange, the cell aggregates were re-seeded into their original PBS-mini vessel in alpha-MEM poor media (MEM alpha media base (MEM alpha, GlutaMAX™, no nucleosides, Gibco ref 32561-.37), supplemented with Gentamicin (Gibco ref 15750060, final concentration (fc)=0.025 mg/mL), and glucose supplement (Gibco ref A2494001, at a ratio of 1:200), filter sterilized using a 0.2 μm filter (ThermoScientific ref 567-0020)), at the same volumes as their day+0 volumes. The cell aggregates were then cultured (37° C., 5% CO₂, at atmospheric oxygen) in suspension, with agitation at 40 rpm for another 2 days, until the end of the vesiculation period (“day+4”).

For Hyperflask adherent culture, fresh single cell CPCs were seeded at 100,000 cells/cm² onto vitronectin-coated hyperflasks in alpha-MEM complete media (“day+0”). In addition, cryopreserved CPCs were thawed at 37° C. for 3 min, transferred to an empty conical tube, then resuspended (dropwise) in alpha-MEM complete media. The thawed cell suspensions were centrifuged, and the cell pellets were resuspended in alpha-MEM complete media. The thawed CPCs were seeded at 100,000 cells/cm² onto vitronectin-coated hyperflasks in alpha-MEM complete media (“day+0”). The seeded cells for both fresh and thawed CPCs were then cultured (37° C., 5% CO₂, at atmospheric oxygen) for 2 days (until “day+2”). At day+2, spent media was removed, and the flasks were rinsed three times with 50-100 mL of pre-warmed MEM alpha media base. The culture vessels were then filled with alpha-MEM poor media, according to the manufacturer's directions, and incubated for 2 more days (37° C., 5% CO₂, at atmospheric oxygen) until the end of the vesiculation period (“day+4”).

At day+2 and day+4, cells in the suspension cultures were counted as described above in Example 1. At day+4, cells in the adherent cultures were harvested by 1/rinsing the cells with DPBS, 2/incubating cells with 100 mL of pre-warmed 0.05% Trypsin-EDTA (Gibco, 15400-054, diluted in DPBS) for 2-3 minutes at room temperature, 3/quenching the harvest with 100 mL aMEM+glutamax supplemented with B27 (minus insulin) (f.c. 1×), 4/collecting the bulk cell suspension into a 500 mL conical centrifuge tube, 5/rinsing harvested flasks with basal aMEM media to recover any remaining cells and adding this rinse to the bulk cell suspension. The concentration of cells in the suspensions were determined using the ViCell Automated Cell Counter, and the cells per cm² from the harvested vessels were back-calculated.

In addition to the CPC adherent and suspension vesiculation cultures, virgin media controls were also performed for adherent and suspension cultures.

For the suspension vesiculation culture virgin media controls, new 0.5 L PBS-mini vessels were filled with 400 mL alpha-MEM complete media (at “day+0”), and incubated for 2 days (37° C., 5% CO₂, at atmospheric oxygen), with agitation at 40 rpm. After the two days (“day+2”), the spent culture media was removed, and vessels were rinsed thoroughly (three times each with 50-100 mL of pre-warmed MEM alpha media base). The PBS-mini vessels were then filled with 400 mL alpha-MEM poor media, and incubated for 2 more days (37° C., 5% CO₂, at atmospheric oxygen), until “day+4.”

For the adherent vesiculation culture virgin media controls, vitronectin-coated hyperflasks were filled with alpha-MEM complete media and incubated for 2 days (37° C., 5% CO₂, at atmospheric oxygen). After these two days (“day+2”), the spent culture media was removed, and the vessels were rinsed thoroughly (three times each with 50-100 mL of pre-warmed MEM alpha media base). The hyperflasks were then filled with alpha-MEM poor media, and incubated for 2 more days (37° C., 5% CO₂, at atmospheric oxygen), until “day+4.”

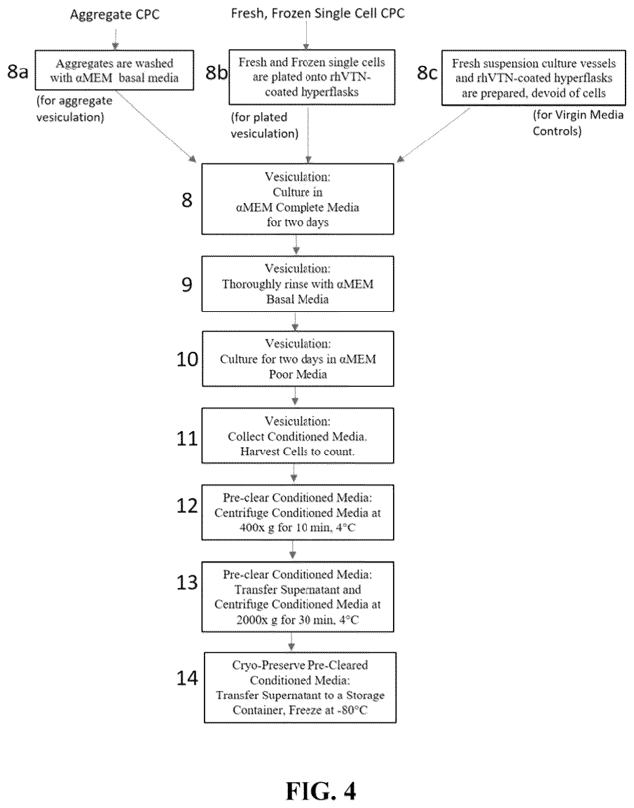

At day+4, media from the suspension and adherent cell cultures (conditioned media, MC), as well as day+4 media from the virgin control vessels (virgin media, MV), were collected, and pre-cleared by serial centrifugation (400×g for 10 minutes at 4° C., then 2000×g for 30 minutes at 4° C.). The pre-cleared media was then aliquoted into conical tubes, and frozen at −80° C. FIG. 4 depicts a process flow diagram for the generation of conditioned media and virgin media controls.

### Example 3

### Preparation of Small Extracellular Vesicle-Enriched Fraction (sEV)

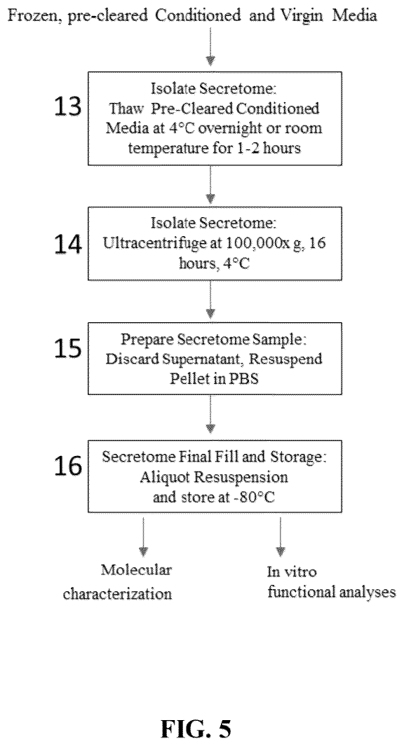

To validate the vesiculation process, samples of the conditioned and control media were subjected to ultracentrifugation, in order to generate sEV and MV preparations for molecular characterization and in vitro functional analyses. Two biological replicates of each sample type were prepared. FIG. 5 depicts a process flow diagram for the isolation of sEV or mock (virgin media) control samples.

MC and MV were thawed at room temperature for 1-4 hours, or overnight at 4° C. After thawing, MC and MV were ultracentrifuged at 100,000×g for 16 hours at 4° C. (wX+Ultra Series Centrifuge, ThermoScientific; rotor: F50 L-8x39; Acceleration: 9; Deceleration: 9), and the resulting supernatants were removed. The bottom of each tube was rinsed twice with 100 volumes of 0.1 μm filtered DPBS−/− (0.1 μm PES Filter Unit, ThermoFisher 565-0010) without disturbing the pellet, and then each pellet was resuspended in 0.1 μm filtered DPBS−/− by gentle agitation of the solvent with a sterilized glass stir bar. sEV preparations were collected, and tubes were rinsed with 0.1 μm filtered DPBS−/− for maximum product recovery (to a total resuspension plus rinse target volume as calculated based on the number of secreting cells giving rise to the conditioned media). 45 μL were targeted for every 1.4×10⁶ day+4 secreting cells as calculated by the following formula:

Target sEV Resuspension Volume=(Total Viable Cells at day+4÷Total Volume Conditioned Media at day+4)×Volume MC Centrifuged×(45 μL±1.4×10⁶ Viable Cells).

Target resuspension volumes for MV controls were matched to the relevant MC target resuspension volumes. For MC and MV generated in PBS-mini vessels, sEV preparations were filtered at 0.65 μm (Ultrafree 0.65 μm DV Durapore, Millipore ref: UFC30DV05) to remove large particulates. sEV and MV control preparations were aliquoted and frozen at −80° C.

sEV and MV control preparations were further analyzed, as described below.

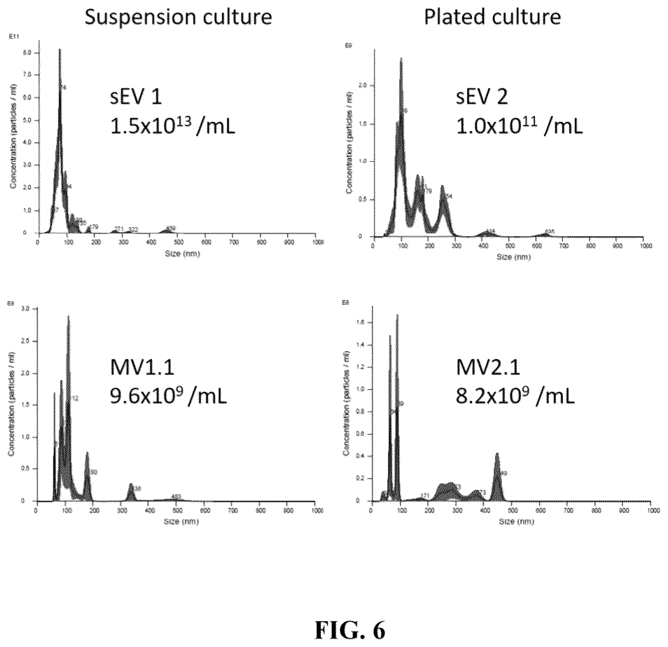

First, the particle concentration and size distribution in sEV and MV control preparations were determined by nanoparticle tracking analysis (NTA; NanoSight). The nanoparticle tracking analysis confirmed the presence of particles of the size of exosomes and microparticles in the sEV prepared from CPC conditioned media, but not in MV controls. FIG. 6 depicts representative size distribution curves from two sEVs and two control MV samples. Observable particle sizes ranged from approximately <30 nm to 300 nm or so, with a peak generally between 50-150 nm, corresponding to the size of exosomes or small microparticles.

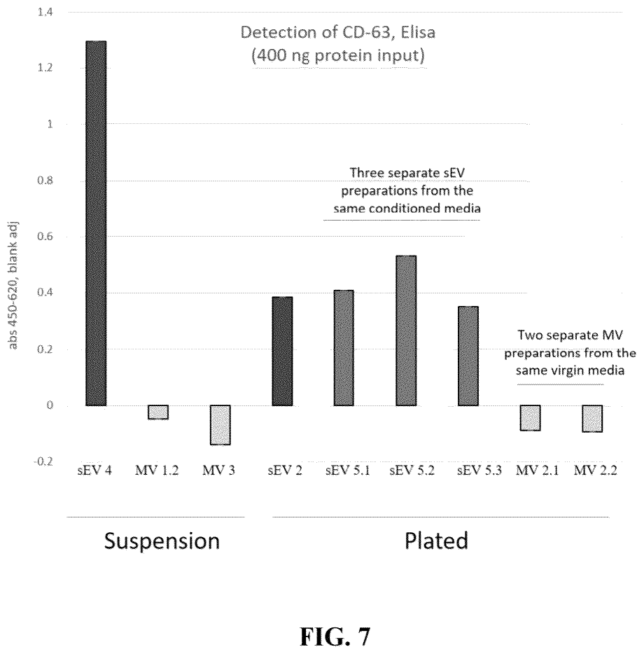

Second, the presence of the exosome-associated vesicle surface marker CD63 was also analyzed using the PS Capture Exosome ELISA Kit (Wako Chemicals, ref: 293-77601), with the primary antibody being an anti-CD63 antibody (Wako Chemicals, ref: 292-79251), and the secondary antibody being an HRP-conjugated Anti-mouse IgG antibody (Wako Chemicals, ref: 299-79261). Input volumes were set such that 400 ng protein from sEV and MV control preparations was added to each well. This anti-CD63 ELISA evaluation confirmed the presence of exosome-associated CD63 surface antigen in each of the sEV samples, but in none of the MV controls (FIG. 7). CD63 signal was higher in the aggregate sample than in the plated samples, although the CD63 signal was consistent between replicates of plated samples. The protein content of sEV and MV control preparations was determined by BCA analysis, using the Pierce Micro BCA kit (ThermoScientific ref: 23235).

### Example 4

### In Vitro Analysis of sEV Functionality

To analyze the functionality of the sEV preparations, three in vitro assays were used: a HUVEC scratch wound healing assay; a cardiomyocyte viability assay using serum-deprived H9c2 cells; and a cardiomyocyte viability assay using staurosporine-treated human cardiomyocytes.

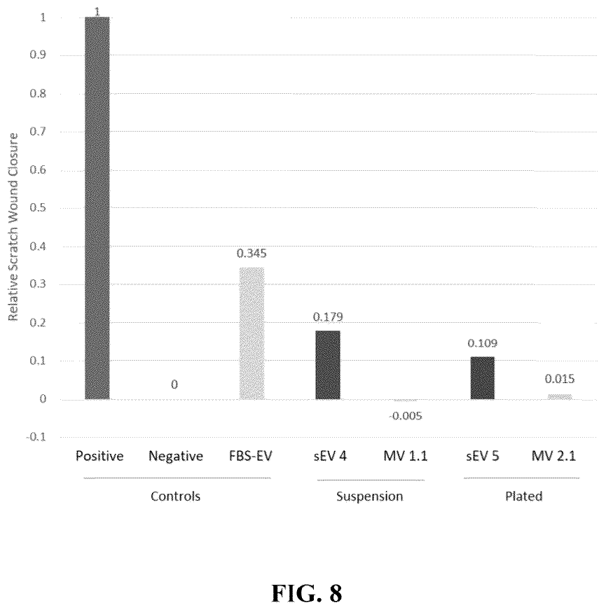

For the HUVEC scratch wound healing assay, a scratch wound healing assay (developed by Essen BioSciences, for the Incucyte) was employed, according to the manufacturer's directions. Briefly, HUVEC cells were expanded using HUVEC Complete Media: Endothelial Cell Basal Media (PromoCell, Ref: C-22210), supplemented with the Endothelial Cell Growth Medium Supplement Pack (PromoCell, Ref: C-39210). After expansion, the cells were cryopreserved in CS10 (Cryostore, ref: 210102) at 1-2×10⁶ cells per aliquot (enough for between a half to a full 96-well plate). Two days prior to assay, HUVEC aliquots were thawed, and plated onto ImageLock 96-well plates (EssenBio, Ref: 4379) at 10,000 cells/well, and grown in HUVEC Complete media for two days. Cultures were maintained at 37° C. (atmospheric oxygen, 5% CO₂) throughout maintenance and assay process. Wells were scratched using a Wound Maker (EssenBio, Ref: 4493) according the manufacturer's directions, and cells were then rinsed with Endothelial Cell Basal Media and cultured overnight (either in HUVEC Complete Media alone, as a positive control; in Endothelial Cell Basal Media alone, as a negative control; or in Endothelial Cell Basal Media supplemented with sEV or MV preparations). Using an Incucyte with the Scratch Wound Healing Module, plates were imaged every three hours for a total of 18 hours. Wound closure was determined using the manufacturer's software, and values were baseline (negative control) subtracted, and normalized to the positive control. FIG. 8 depicts that the sEV preparations, but not the control MV preparation, promoted wound healing, indicating the functionality of the sEV preparation.

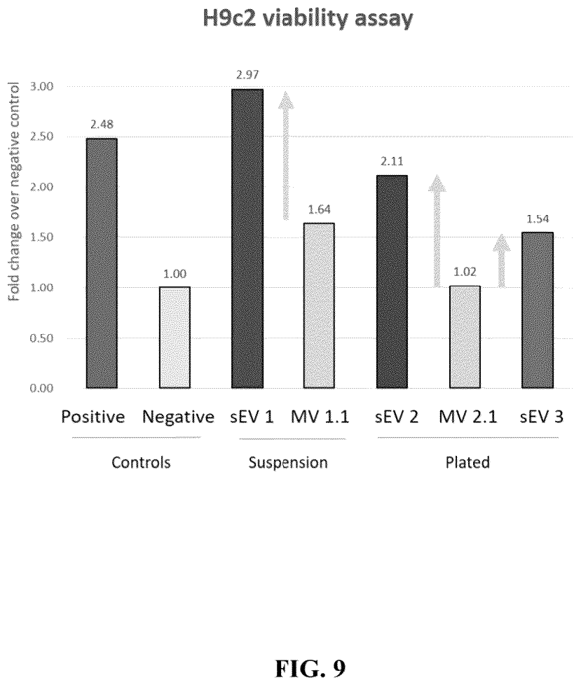

For the cardiomyocyte viability assay using serum-deprived H9c2 cells, the assay was performed essentially as described in El Harane et al. (Eur. Heart J., 2018; 39:1835-1847). In this assay, H9c2 cardiomyocytes are proliferative when culture media is rich in serum (e.g., cultured in H9c2 Complete Media), but cease to proliferate and loose viability when they are deprived of serum (e.g., cultured in H9c2 Poor Media). The capacity of sEV and MV preparations to promote H9c2 cardiomyocyte viability was determined by supplementing the H9c2 Poor Media with increasing concentrations of sEV and MV control preparations. FIG. 9 depicts that the sEV preparations, but not the control MV preparation, improved H9c2 cardiomyocyte viability in the absence of serum, indicating the functionality of the sEV preparation.

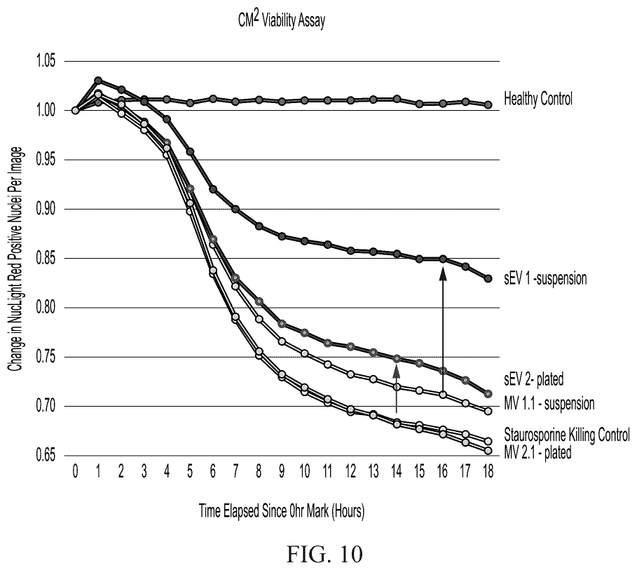

For the cardiomyocyte viability assay using staurosporine-treated human cardiomyocytes, iCell Cardiomyocytes2 (Fujifilm Cellular Dynamics, Inc., ref: CMC-100-012-001) were plated at 50,000 cells/well of a fibronectin-coated 96-well plate in iCell Cardiomyocyte Plating Medium (Fujifilm Cellular Dynamics, Inc., ref: M1001), and cultured for 4 hours. The media was then exchanged for iCell Cardiomyocyte Maintenance Medium (iCMM, Fujifilm Cellular Dynamics, Inc., ref: M1003), and cells were cultured for up to 7 days, with full media exchanges every 2-3 days. After a minimum of 4 days, cells were exposed to iCMM with NucSpot Live 650 dye (Biotium, ref: 40082) (this served as a viable cell control); or to iCMM with NucSpot Live 650 dye, and staurosporine (Abcam, ref: ab146588) at a final in-well concentration of 2 μM (this also served as an apoptotic cell control). Dye, PBS, and DMSO concentrations, and final well volumes, were equivalent in all wells. Cells were cultured in these pre-incubation media for four hours. After this incubation, the pre-incubation media was removed, and the wells were rinsed with iCMM. Cells were then fed with iCMM with NucSpot Live 650 dye and PBS, or iCMM with NucSpot Live 650 dye supplemented with increasing concentrations of sEV or MV control preparations while maintaining PBS final volumes. Wells were imaged in an Incucyte every hour for 24 hours, and nuclei counts were determined. FIG. 10 depicts that the sEV preparations, but not the control MV preparation, improved cardiomyocyte survival, indicating the functionality of the sEV preparation.

### Example 5

### First Exemplary Good Manufacturing Practices (GMP)-Compatible Process for Producing Small Extracellular Vesicle-Enriched Fraction (sEV) Formulations

A first exemplary GMP-compatible process for producing sEV-containing formulations was developed. The production process included four main stages: vesiculation; conditioned media clarification; enrichment and concentration of small EV-enriched secretome; and production of the final sEV formulation. Flow diagrams outlining the GMP-compatible process that was performed are depicted in FIGS. 11A and 11B.

Vesiculation

For the vesiculation step, cardiovascular progenitor cells (CPCs) that had been cryopreserved and stored under vapor-phase liquid nitrogen (or within a −150° C. freezer) were initially thawed for two minutes at 37° C. in a thawing medium (MEM alpha (MEM a, GlutaMAX™ Supplement, no nucleosides; Gibco/Life Technologies; ref: 32561-029); glucose (30%) supplement (Macopharma Ref: CARELIDE, to a final overall glucose concentration of 2 mg/mL; Ydralbum® (LFB), at a final concentration of 20 mg/mL; B-27™ Supplement (50×, Life Tech Ref: 17504001 at a final concentration of 1×); and Rock Inhibitor H1152 (Sigma Ref: 555550, at a final concentration of 0.392 μg/mL), within an EVA bag (Corning). 18 mL of thawing medium was used per 1 mL of CPCs.

After thawing, CPCs were seeded onto vitronectin (Life Tech Ref: VTN-N; recombinant human protein, truncated (Ref: A31804); 5 μg/mL, sterilized using a 0.22 μm filter (syringe filter 0.2 μm polyethersulfone (PES) membrane) coated culture flasks (8×10ST CellStack Culture Chambers, tissue culture (TC)-treated (Corning Ref: 3271); as well as 2×TC-treated, vitronectin-coated T75 flasks), at a seeding density of about 100,000 cells per cm², using 0.2 mL/cm² of complete medium (MEM a, GlutaMAX™ Supplement, no nucleosides; Gibco/Life Technologies; ref: 32561-029; glucose (30%) supplement (Macopharma Ref: CARELIDE, to a final overall glucose concentration of 2 mg/mL; Ydralbum® (LFB; 200 g/L); B-27™ Supplement (50×, Life Tech Ref: 17504001 or 17504044, at a final concentration of 1×); Gentamicin (Panpharma, at a final concentration of 25 μg/mL); and Human FGF-2 Premium grade (Miltenyi Biotec ref: A12873-01, at a final concentration of 1 μg/mL)). Seeding was performed without prior centrifugation of the cell suspension. The seeded CPCs were then cultured in complete medium for three days at 37° C., in the presence of 5% CO₂ and atmospheric oxygen.

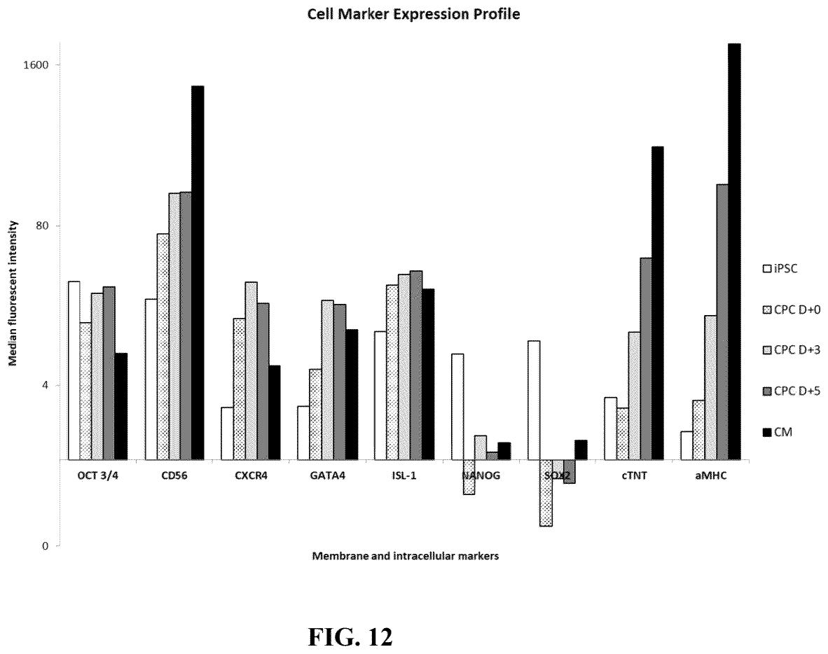

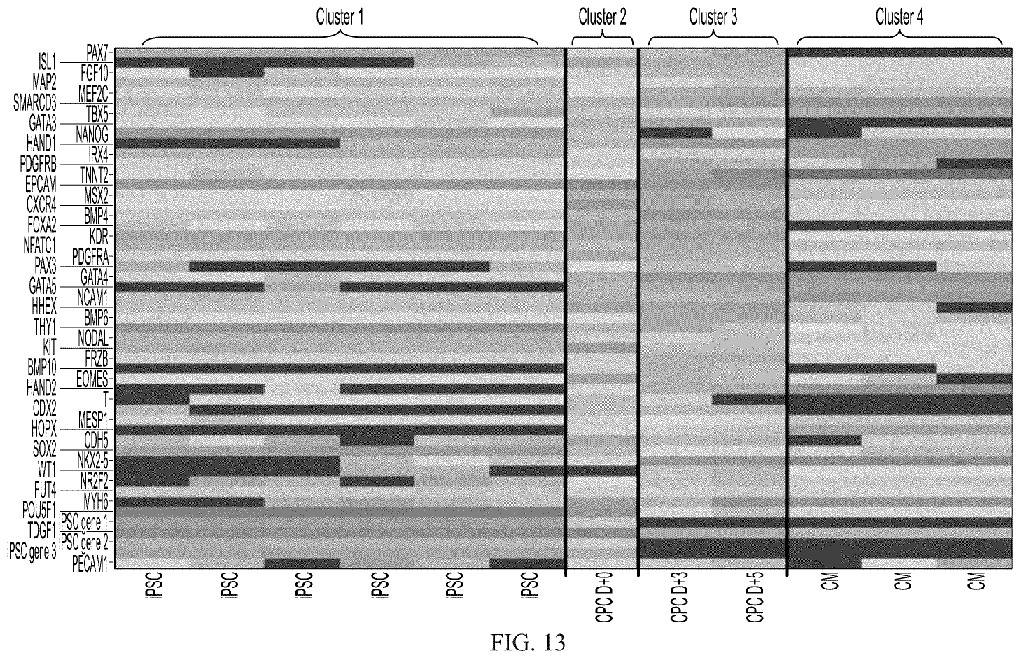

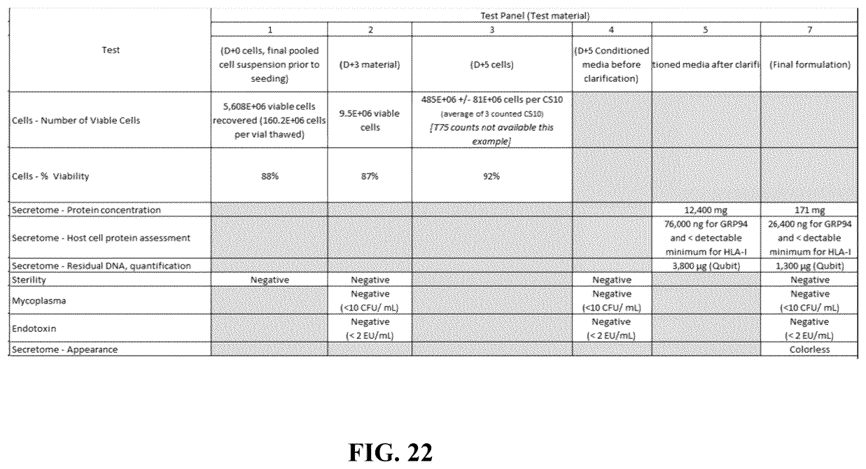

Immediately prior to seeding (“D+0”), cells were analyzed to determine the number and percentage of viable cells (see FIG. 22, column 1 (“D+0 cells”) using a NucleoCounter NC-200 (Chemometec) with DAPI/AO staining (Ph. Eur. 2.7.29); to determine their identity (see FIG. 12 and Example 7) by flow cytometry using a MACSQuant 10 Flow Cytometer; and to analyze their transcriptome (see FIG. 13 and Example 8).

After the 3-day culturing (“D+3”), the cells from one of the cultured T75 flasks were harvested. These harvested cells were analyzed to determine the number and percentage of viable cells (see FIG. 22, column 2 (“D+3 material”) using a NucleoCounter NC-200 (Chemometec) with DAPI/AO staining (Ph. Eur. 2.7.29); to determine their identity (see FIG. 12 and Example 7) by flow cytometry using a MACSQuant 10 Flow Cytometer; and to analyze their transcriptome (see FIG. 13 and Example 8). Spent media from the 10ST CellStack Culture Chambers was also tested for sterility, and for the presence of mycoplasma and endotoxin.

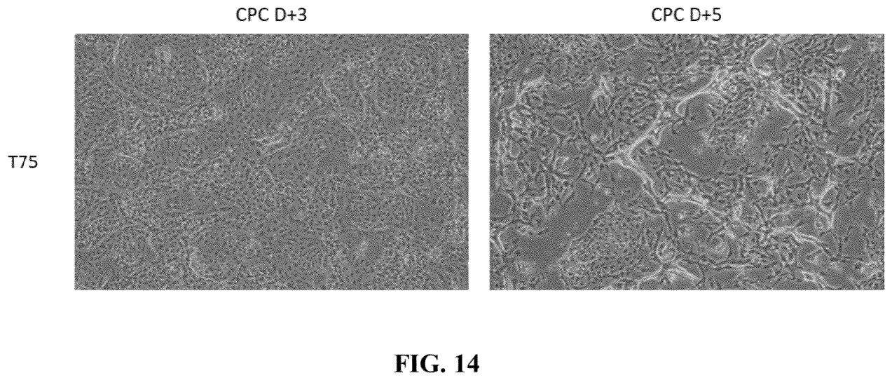

For the remaining flasks (8×10ST CellStack Culture Chambers; and 1×T75), the cells were visualized by microscopy to determine their morphology (see FIG. 14), and washed twice with a wash medium (MEM alpha (Macopharma Ref: BC0110021); glucose (30%) supplement (Macopharma Ref: CARELIDE, to a final overall glucose concentration of 2 mg/mL), before being cultured for 2 days at 37° C., in the presence of 5% CO₂, in a starvation media (poor media) (MEM alpha (1000 mL of Macopharma Ref: BC0110021); glucose (30%) supplement (Macopharma Ref: CARELIDE, to a final overall glucose concentration of 2 mg/mL). After this 2-day incubation (“D+5”), the culture media (conditioned media) was collected, and the cells from the 10ST CellStack Culture Chambers and the remaining T75 flask were harvested.

As with the cells at D+3, the cells at D+5 were again visualized by microscopy to determine their morphology (see FIG. 14); and the cells harvested at D+5 were further analyzed to determine the number and percentage of viable cells (see FIG. 22, column 3 (“D+5 cells”); to determine their identity (see FIG. 12 and Example 7) by flow cytometry using a MACSQuant 10 Flow Cytometer; and to analyze their transcriptome (see FIG. 13 and Example 8). The collected conditioned media was tested for sterility, and for the presence of mycoplasma and endotoxin, before further processing.

Conditioned Media Clarification

Clarification of the conditioned media was conducted via a series of four filtration steps. First, filtration was performed using a 200 μm drip chamber filter (Gravity Blood Set, BD careFusion Ref: VH-22-EGA). The resulting filtrate was then filtered with an infuser, using a 15 μm filter (DIDACTIC, Ref: PER1FL25). The resulting filtrate was then filtered using Sartoguard PES XLG MidiCaps (Pore sizes (prefilter+filter): 1.2 μm+0.2 μm, size 7 (0.065 m²); Sartorius Ref: 5475307F7-OO-A). Next, the resulting filtrate was further filtered using a Vacuum Filter/Storage Bottle System (0.22 μm, Pore 33.2 cm², PES Membrane; Corning Ref: 431097).

Enrichment and Concentration

Following clarification of the conditioned media, the conditioned media was subjected to enrichment and concentration of the small EV secretome.

First, the clarified conditioned media was subjected to Tangential Flow Filtration (TFF), using a TFF Allegro™ CM150 (PALL/Sartorius). For the TFF manifold, a sterile single-use Flow Path Manual Valve P&F (PALL/Sartorius, reference: 744-69N) was used, together with a 5 L Retentate Assembly (sterile, single use; PALL/Sartorius Ref: 744-69 L). For the TFF cassette, sterile single-use regenerated cellulose filters (30 kDa cut-off; 0.14 m²; Sartorius Ref: Opta filter assembly+3D51445901MFFSG) were used. For recovery of the retentate (i.e., what is retained in the TFF), a Bench Top TFF 1 L Bag was used (PALL/Sartorius, reference: 7442-0303P).

Initially, the TFF device was washed with 10 L of H₂O, and 1 L of 1×PBS (filter sterilized using a 0.2 μm filter) before operation. Next, after administration of the clarified conditioned media to the TFF device, the retentate was concentrated (to 500 mL; not exceeding 3 bars of pressure). After this initial concentration step, the retentate was subjected to diafiltration (6 diafiltration volumes; using 1×DPBS, filter sterilized using a 0.2 μM filter). After diafiltration, the retentate was further concentrated, to produce a total volume of at least 100 mL. The parameters of the TFF process were as follows: feed manifold pressure (PT01)—0.86-2.1 bars; retentate manifold pressure (PT02)—0.11-0.14 bars; retentate manifold flow rate (FT01)—0.03-0.32 L/min; transmembrane pressure (TMP01)—0.4-1.1 bars; and quattroflow pump (P01)—18-23%.

### Example 6

### Formulation/Composition

After enrichment and concentration by TFF, retentate was processed as depicted in FIG. 11B. Briefly, retentate alone, retentate including 25 mM trehalose, and retentate including 5 g/L L-histidine, were each stored in glass vials (2 mL, bromobutyl cap; Adelphi Ref: VCDIN2RDLS1) and stored at −80° C. Quality control testing was performed on these samples (the different stages at which quality control testing was undertaken are indicated with a “*,” e.g., *6, *7, etc.). Additionally, final sEV formulations were also prepared by filter sterilizing retentate (with or without 25 mM trehalose) using a 0.22 μm filter (Sterivex™-GP Pressure Filter Unit, 0.22 μm, Millipore, Ref: SVGPL10RC). After the sterilization step, the final formulations (with or without the addition of 25 mM trehalose) were bottled into glass vials (2 mL, bromobutyl cap; Adelphi Ref: VCDIN2RDLS1). Final formulations were stored at −80° C. for future use or testing.

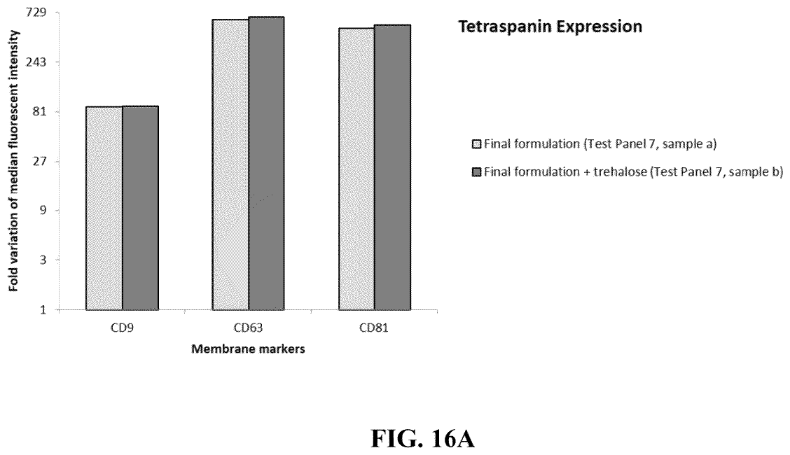

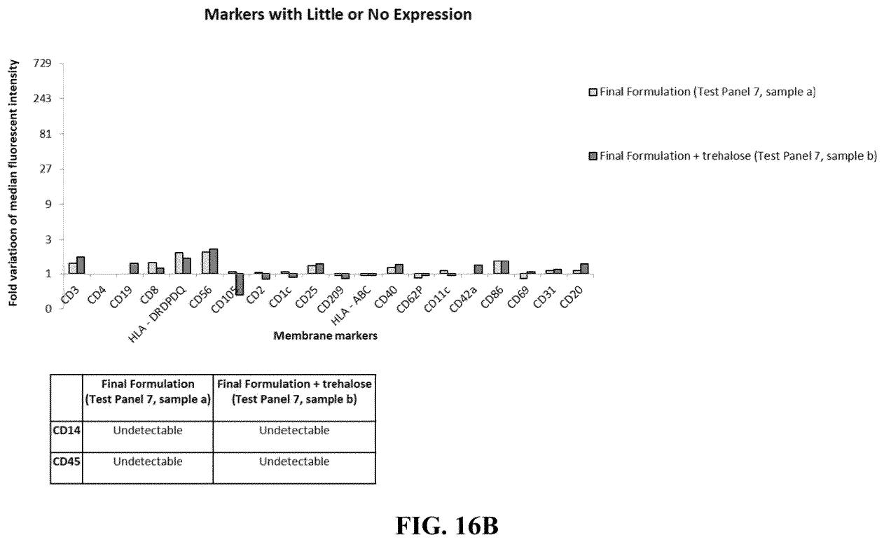

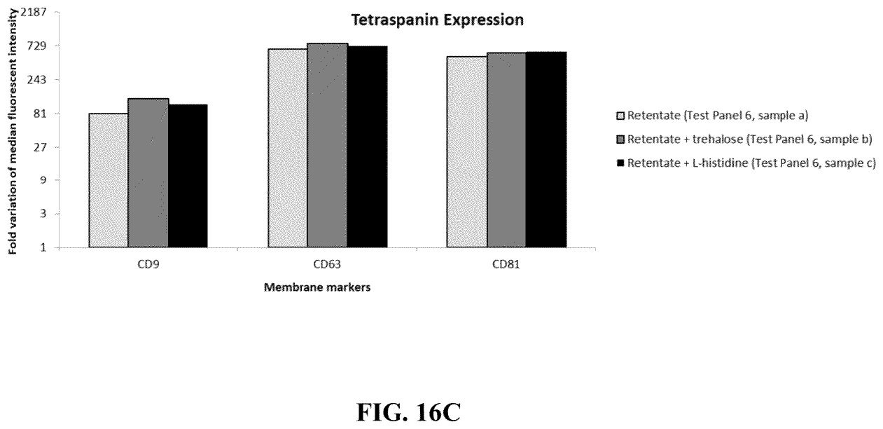

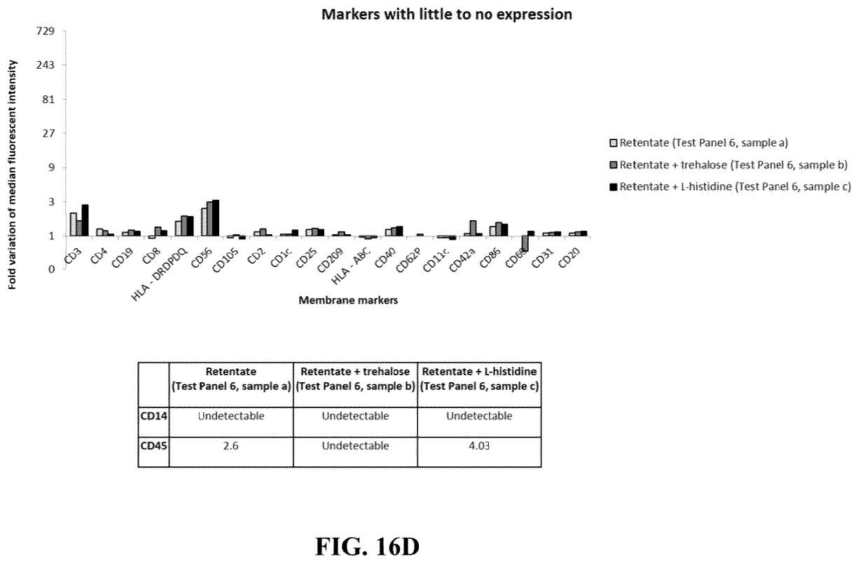

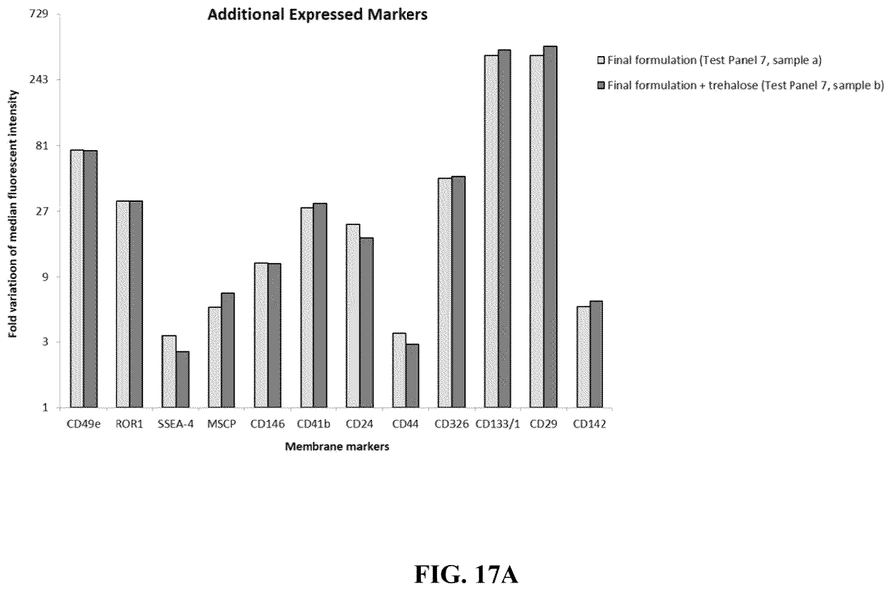

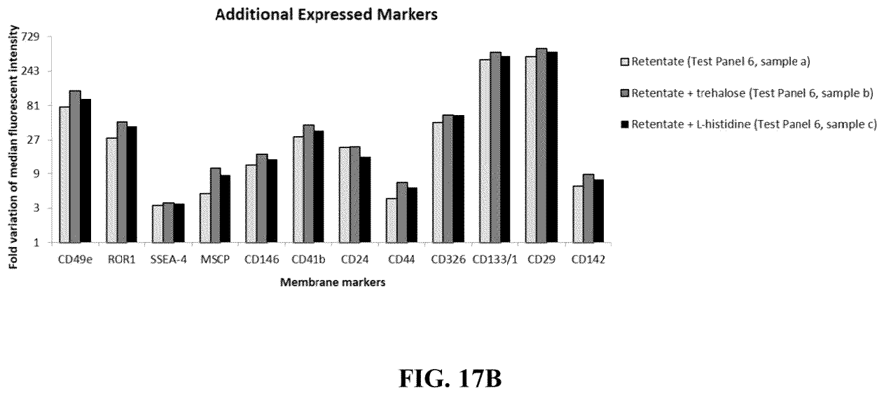

The final formulations, therefore, were in PBS (with or without trehalose), and were positive for CD9, CD63 and CD81 (canonical EV markers), as well as positive for the cardiac-related markers CD49e, ROR1, SSEA-4, MSCP, CD146, CD41b, CD24, CD44, CD236, CD133/1, CD29 and CD142, as detected by MACSPlex (as shown in FIGS. 16A, 16C, 17A and 17B).

### Example 7

### Characterization of the Identity of CPCs During Vesiculation in the GMP-Compatible Process

To assess the identity of the cells during the vesiculation process in Example 5, the D+0 CPCs, as well as the harvested cells at D+3 and D+5, were analyzed by flow cytometry. iPSCs and cardiomyocyte (CM) cells were included as controls. As shown in FIG. 12, flow cytometry analysis, performed using a MACSQuant 10 Flow Cytometer with iPSC-, CPC- and cardiac-markers, demonstrated that the CPCs became more mature over the five-day vesiculation period. Specifically, the CPCs maintained little to no NANOG or SOX2 protein expression, and exhibited a continued increase in CD56, cTNT, and aMHC, protein expression (however, they did not reach expression levels of CD56, cTNT, and aMHC similar to cardiomyocytes, indicating that they remained progenitors throughout the process). iPSC and CM control cells were analyzed separately, and the average values are presented in FIG. 12 for comparative purposes.

### Example 8

### Transcriptome Analysis of CPCs During Vesiculation in the GMP-Compatible Process

To assess the transcriptome of the cells during the vesiculation process in Example 5, RNA was extracted from the CPCs at D+0, and from the harvested cells at D+3 and D+5 of the vesiculation process. RNA was also extracted from iPSCs (pluripotent cell controls), and from iPSC-derived cardiomyocytes (differentiated cardiomyocyte controls). Total RNA was sequenced on the Illumina NovaSeq 6000 platform, and differential gene expression was determined on normalized data.

The heat map depicted in FIG. 13 was generated based on hierarchical clustering analysis using the UPGMA clustering method, with correlation distance metric in TIBCO Spotfire software v11.2.0. The genes included in the panel included genes expressed at different stages of differentiation (from iPSC through to beating cardiomyocytes), as well as related off-target cells. The gene expression analysis results depicted in FIG. 13 thus confirmed that the cells retained the characteristics of cardiovascular progenitors throughout the vesiculation process.

### Example 9

### Analysis of EV Particle Concentration and EV Particle Size Distribution in the GMP-Compatible Process

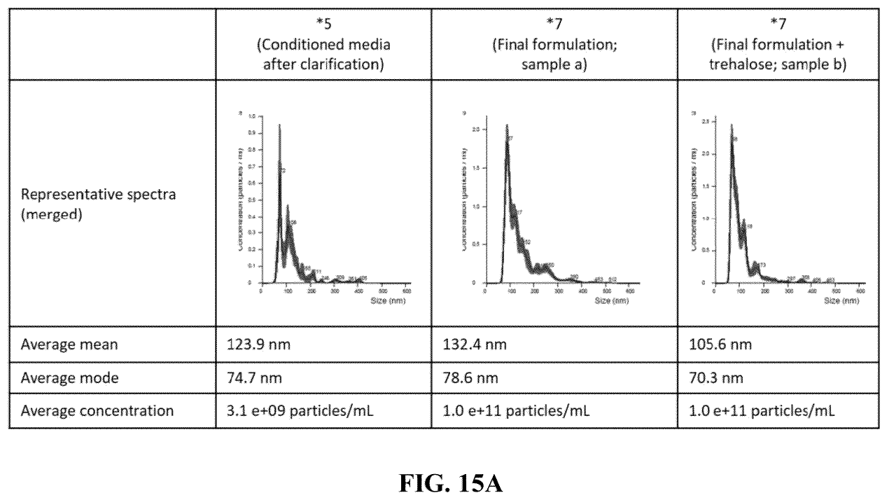

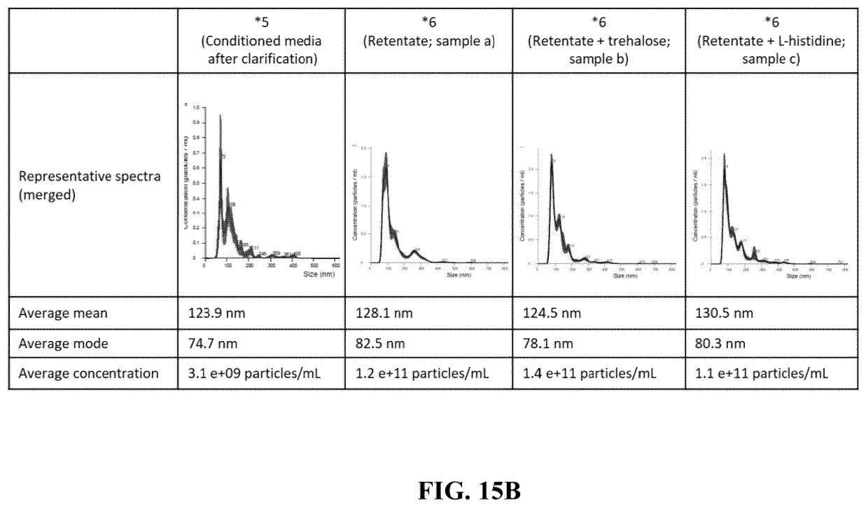

To assess the particle concentration and size distribution of EVs produced in Example 5, the clarified conditioned media (before TFF), and the final formulations (with and without trehalose), were analyzed by nanoparticle tracking analysis (NTA; NanoSight). FIG. 15A depicts representative size distribution curves for each sample. The overall size distributions, means and modes, were similar between samples. A peak was observed generally between 50-150 nm, corresponding to the size of exosomes or small microparticles. The TFF step resulted in an approximately 32-fold concentration of particles. Similar experiments were also conducted on the stored retentate samples depicted in FIG. 11B (with and without trehalose or histidine) which were not filter sterilized (“6,” samples a-c). The results of these experiments are shown in FIG. 15B.

### Example 10

### Analysis of EVs Produced by the GMP-Compatible Process for EV Markers

To assess the presence of EV markers in the clarified conditioned media (before TFF) and the final formulations (with and without trehalose) in Example 5, a MACSPlex Exosome Kit human (Miltenyi Ref: 130-108-813) was used to identify and quantify the presence of EV markers. As shown in FIG. 16A, the analysis confirmed the presence of extracellular vesicle tetraspanins (CD9, CD81 and CD63) in both the conditioned media (before TFF), and in the final formulation (with and without trehalose). Further still, as shown in FIG. 16B, the MACSPlex analysis also revealed a variety of markers that were found to be present either in low amounts (e.g., CD3, CD4, CD8, HLA-DRDPDQ, CD56, CD105, CD2, CD1c, CD25, CD40, CD11c, CD86, CD31 and CD20); or were substantially absent (CD19, CD209, HLA-ABC, CD62P, CD42a and CD69), in the conditioned media (before TFF), and/or in the final formulation (with and without trehalose). Similar experiments were also conducted on the stored retentate samples depicted in FIG. 11B (with and without trehalose or histidine) which were not filter sterilized (“*6,” samples a-c). The results of these experiments are shown in FIGS. 16C and 16D.

Additionally, as shown by FIG. 17A, additional cardiac-related markers were also observed in the conditioned media (before TFF), and in the final formulation (with and without trehalose). Similar experiments were also conducted to confirm the presence of these additional cardiac-related markers in the stored retentate samples depicted in FIG. 11B (with and without trehalose or histidine) which were not filter sterilized (“6,” samples a-c). The results of these experiments are shown in FIG. 17B.

### Example 11

### In Vitro Analysis of the Potency of EVs Produced by the GMP-Compatible Process

To analyze the functionality and potency of the final formulations produced by the GMP-compatible process in Example 5, two in vitro assays were used: a HUVEC scratch wound healing assay; and a cardiomyocyte viability assay using staurosporine-treated human cardiomyocytes.

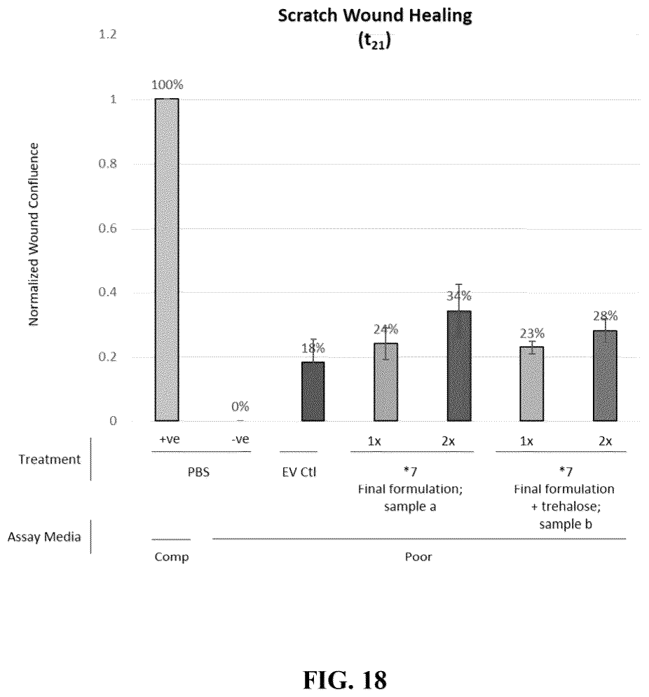

For the HUVEC scratch wound healing assay, a scratch wound healing assay (developed by Essen BioSciences, for the Incucyte) was employed, according to the manufacturer's directions. Briefly, HUVEC cells were expanded using HUVEC Complete Media: Endothelial Cell Basal Media (PromoCell, Ref: C-22210), supplemented with the Endothelial Cell Growth Medium Supplement Pack (PromoCell, Ref: C-39210). After expansion, the cells were cryopreserved in CS10 (Cryostore, ref: 210102) at 1-2×10⁶ cells per aliquot (enough for between a half to a full 96-well plate). Two days prior to assay, HUVEC aliquots were thawed, and plated onto ImageLock 96-well plates (EssenBio, Ref: 4379) at 10,000 cells/well, and grown in HUVEC Complete media for two days. Cultures were maintained at 37° C. (atmospheric oxygen, 5% CO₂) throughout the maintenance and assay process. Wells were scratched using a Wound Maker (EssenBio, Ref: 4493) according to the manufacturer's directions, and cells were then rinsed with Endothelial Cell Basal Media and cultured overnight (either in HUVEC Complete Media and PBS, as a positive control; in Endothelial Cell Basal Media and PBS, as a negative control; or in Endothelial Cell Basal Media supplemented with sEV preparations in PBS). Using an Incucyte with the Scratch Wound Healing Module, plates were imaged at 21 hours after treatment. Wound closure was determined using the manufacturer's software, and values were baseline (negative control) subtracted, and normalized to the positive control. FIG. 18 depicts that the final formulations with and without trehalose (sample b and a, respectively) promoted wound healing.

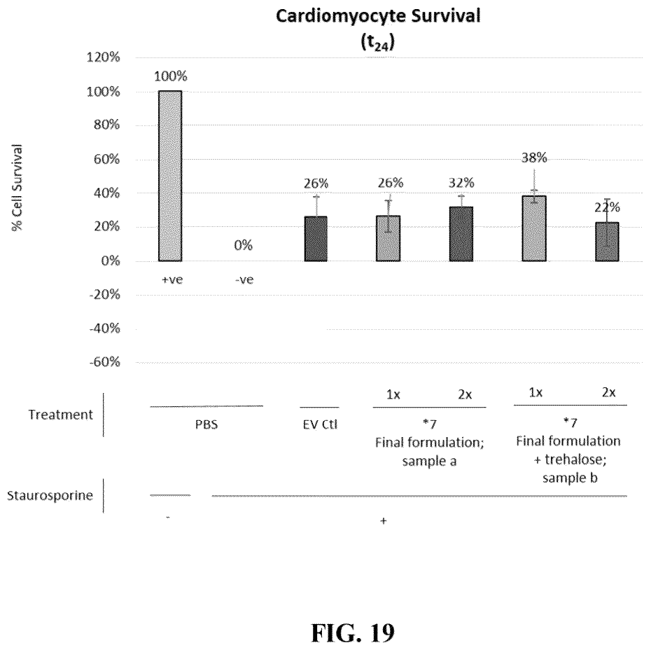

For the cardiomyocyte viability assay using staurosporine-treated human cardiomyocytes, iCell Cardiomyocytes2 (Fujifilm Cellular Dynamics, Inc., ref: CMC-100-012-001) were plated at 50,000 cells/well of a fibronectin-coated 96-well plate in iCell Cardiomyocyte Plating Medium (Fujifilm Cellular Dynamics, Inc., ref: M1001), and cultured for 4 hours. The media was then exchanged for iCell Cardiomyocyte Maintenance Medium (iCMM, Fujifilm Cellular Dynamics, Inc., ref: M1003), and cells were cultured for up to 7 days, with full media exchanges every 2-3 days. After a minimum of 4 days, cells were exposed to iCMM with NucSpot Live 650 dye (Biotium, ref: 40082) (this served as a viable cell control); or to iCMM with NucSpot Live 650 dye, and staurosporine (Abcam, ref: ab146588) at a final in-well concentration of 2 μM (this also served as an apoptotic cell control). Dye, PBS, and DMSO concentrations, and final well volumes, were equivalent in all wells. Cells were cultured in these pre-incubation media for four hours. After this incubation, the pre-incubation media was removed, and the wells were rinsed with iCMM. Cells were then fed with iCMM with NucSpot Live 650 dye and PBS, or iCMM with NucSpot Live 650 dye supplemented with increasing concentrations of sEV preparations (sample a and b) while maintaining PBS final volumes. Wells were imaged in an Incucyte at 24 hours, and nuclei counts were determined. FIG. 19 depicts that the final formulations with and without trehalose promoted cardiomyocyte survival.

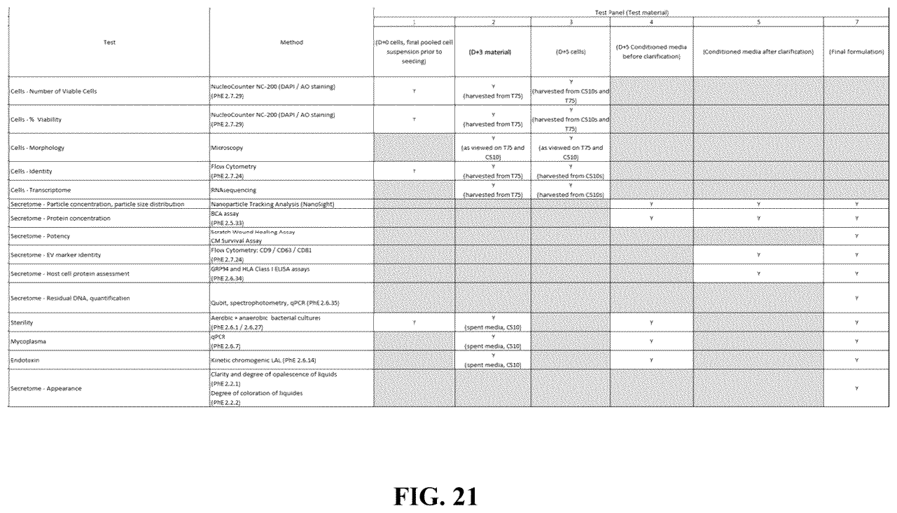

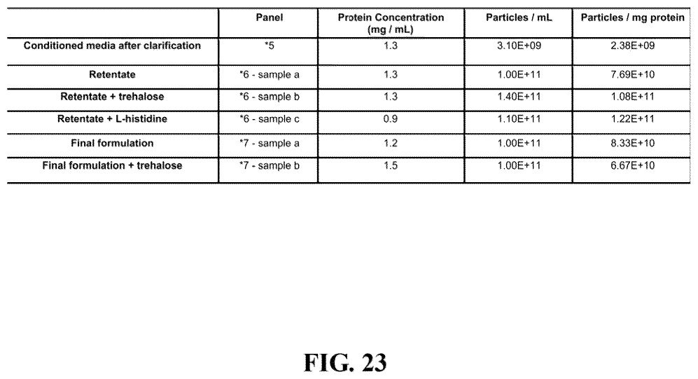

The testing panel used with respect to the processes/products of Example 5, and as embodied, e.g., in Examples 6-11, is shown in FIG. 21. Results therefore are shown in FIG. 22. Additionally, FIG. 23 depicts the degree of enrichment, as compared to conditioned media after clarification, for the retentates and final formulations produced in Example 6.

### Example 12

### Second Exemplary Good Manufacturing Practices (GMP)-Compatible Process for Producing Small Extracellular Vesicle-Enriched Fraction (sEV) Formulations

A second exemplary GMP-compatible process for producing sEV-containing formulations was developed. The production process included four main stages: vesiculation; conditioned media clarification; enrichment and concentration of small EV-enriched secretome; and production of the final sEV formulation. Flow diagrams outlining the GMP-compatible process that was performed are depicted in FIGS. 24A and 24B.

Vesiculation

For the vesiculation step, cardiovascular progenitor cells (CPCs) that had been cryopreserved and stored under vapor-phase liquid nitrogen (or within a −150° C. freezer) were initially thawed for 2.5 minutes at 37° C. in a thawing medium (MEM alpha (1000 mL of Macopharma Ref: BC0110021); glucose (30%) supplement (Macopharma Ref: CARELIDE, to a final overall glucose concentration of 2 mg/mL; Ydralbum® (LFB), at a final concentration of 20 mg/mL; B-27™ Supplement (50×, Life Tech Ref: 17504001 at a final concentration of 1×); and Rock Inhibitor H1152 (Sigma Ref: 555550, at a final concentration of 0.392 μg/mL, sterilized using a 0.2 μm cellulose acetate (CA) membrane syringe filter), within an EVA bag (Corning). 18 mL of thawing medium was used per 1 mL of CPCs.

After thawing, CPCs were seeded onto vitronectin (Life Tech Ref: VTN-N; recombinant human protein, truncated (Ref: A31804); 5 μg/mL, sterilized using a 0.2 μm cellulose acetate (CA) membrane syringe filter) coated culture flasks (12×10ST CellStack Culture Chambers, tissue culture (TC)-treated (Corning Ref: 3271); as well as 2×TC-treated, vitronectin-coated T75 flasks), at a seeding density of about 100,000 cells per cm², using 0.2 mL/cm² of complete medium (MEM alpha (1000 mL of Macopharma Ref: BC0110021); glucose (30%) supplement (Macopharma Ref: CARELIDE, to a final overall glucose concentration of 2 mg/mL; Ydralbum® (LFB; 200 g/L); B-27™ Supplement (50×, Life Tech Ref: 17504001 or 17504044, at a final concentration of 1×); Gentamicin (Panpharma, at a final concentration of 25 μg/mL); and Human FGF-2 Premium grade (Miltenyi Biotec ref: A12873-01, at a final concentration of 1 μg/mL, sterilized using a 0.2 μm cellulose acetate (CA) membrane syringe filter)). Seeding was performed without prior centrifugation of the cell suspension. The seeded CPCs were then cultured in complete medium for three days at 37° C., in the presence of 5% CO₂ and atmospheric oxygen.

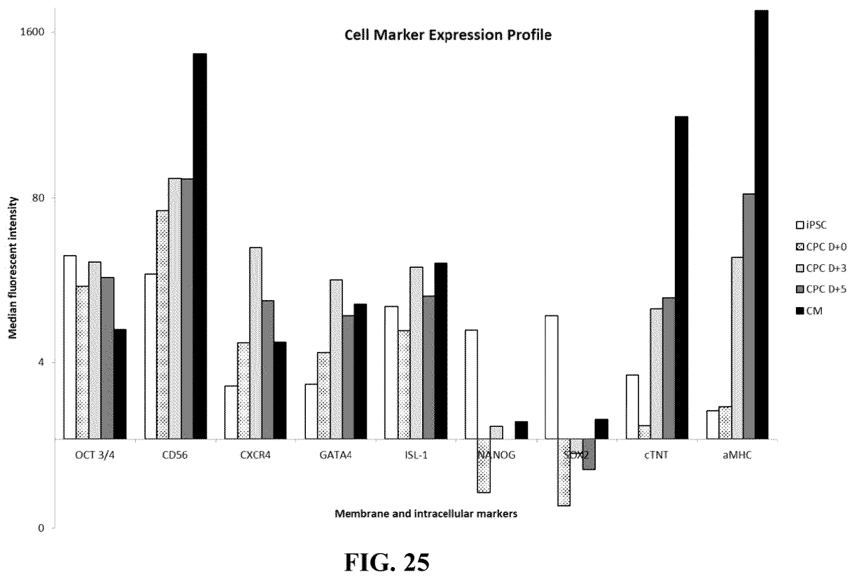

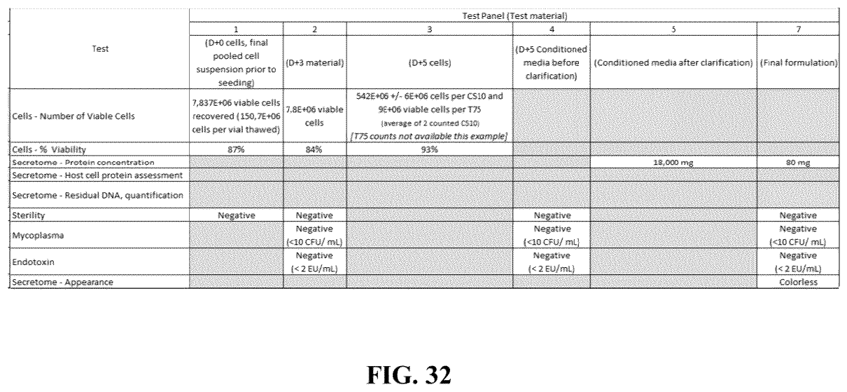

Immediately prior to seeding (“D+0”), cells were analyzed to determine the number and percentage of viable cells (see FIG. 32, column 1 (“D+0 cells”) using a NucleoCounter NC-200 (Chemometec) with DAPI/AO staining (Ph. Eur. 2.7.29); to determine their identity (see FIG. 25 and Example 14) by flow cytometry using a MACSQuant 10 Flow Cytometer.

After the 3-day culturing (“D+3”), the cells from one of the cultured T75 flasks were harvested. These harvested cells were analyzed to determine the number and percentage of viable cells (see FIG. 32, column 2 (“D+3 material”) using a NucleoCounter NC-200 (Chemometec) with DAPI/AO staining (Ph. Eur. 2.7.29); and to determine their identity (see FIG. 25 and Example 14) by flow cytometry using a MACSQuant 10 Flow Cytometer. Spent media from the 10ST CellStack Culture Chambers was also tested for sterility, and for the presence of mycoplasma and endotoxin.

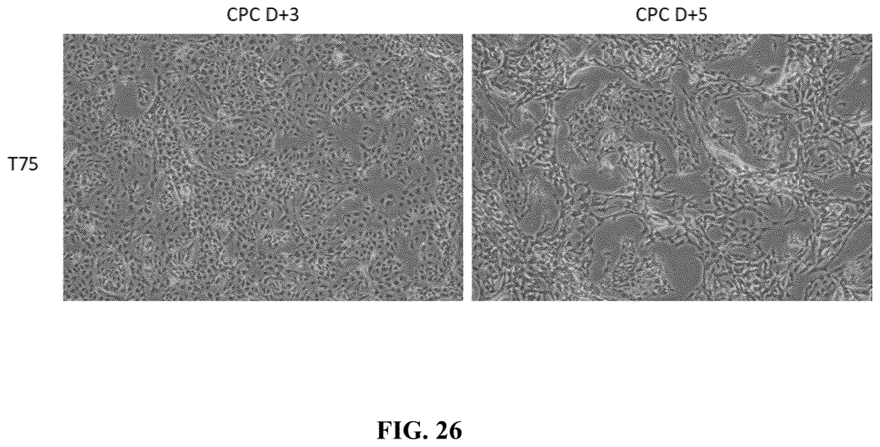

For the remaining flasks (12×10ST CellStack Culture Chambers; and 1×T75), the cells were visualized by microscopy to determine their morphology (see FIG. 26), and washed twice with a wash medium (MEM alpha (1000 mL of Macopharma Ref: BC0110021); glucose (30%) supplement (Macopharma Ref: CARELIDE, to a final overall glucose concentration of 2 mg/mL), before being cultured for 2 days at 37° C., in the presence of 5% CO₂ and atmospheric oxygen, in a starvation media (poor media) (MEM alpha (1000 mL of Macopharma Ref: BC0110021); glucose (30%) supplement (Macopharma Ref: CARELIDE, to a final overall glucose concentration of 2 mg/mL). After this 2-day incubation (“D+5”), the culture media (conditioned media) was collected, and the cells from the 10ST CellStack Culture Chambers and the remaining T75 flask were harvested.

As with the cells at D+3, the cells at D+5 were again visualized by microscopy to determine their morphology (see FIG. 26); and the cells harvested at D+5 were further analyzed to determine the number and percentage of viable cells (see FIG. 32, column 3 (“D+5 cells”); and to determine their identity (see FIG. 25 and Example 14) by flow cytometry using a MACSQuant 10 Flow Cytometer. The collected conditioned media was tested for sterility, and for the presence of mycoplasma and endotoxin, before further processing.

Conditioned Media Clarification

Clarification of the conditioned media was conducted via a series of three filtration steps. First, filtration was performed using a Sartopure PP3 MidiCaps 5 μm PES filter (Sartorius, Ref: 5055342P9-OO-A (Sartorius)). The resulting filtrate was then filtered using a Sartoguard PES MidiCaps filter (Pore sizes (prefilter+filter): 1.2 μm+0.2 μm; Sartorius Ref: 5475307F9-OO-A). The resulting filtrate was then filtered using a Sartopure 2 MidiCaps filter (Pore sizes (prefilter+filter): 0.45 μm+0.2 μm; Sartorius Ref: 5445307H8-OO-A).

Enrichment and Concentration

Following clarification of the conditioned media, the conditioned media was subjected to enrichment and concentration of the small EV secretome.

First, the clarified conditioned media was subjected to Tangential Flow Filtration (TFF), using a TFF Allegro™ CM150 (PALL/Sartorius). For the TFF manifold, a sterile single-use Flow Path Manual Valve P&F (PALL/Sartorius, reference: 744-69N) was used, together with a 10 L Retentate Assembly (sterile, single use; PALL/Sartorius Ref: 744-69M). For the TFF cassette, sterile single-use regenerated cellulose filters (30 kDa cut-off; 0.14 m²; Sartorius Ref: Opta filter assembly+3D51445901MFFSG) were used. For recovery of the retentate (i.e., what is retained in the TFF), a Bench Top TFF 1 L Bag was used (PALL/Sartorius, reference: 7442-0303P).

Initially, the TFF device was washed with 10 L of H₂O, and 2 L of 1×PBS before operation. Next, after administration of the clarified conditioned media to the TFF device, the retentate was concentrated (to 500 mL; not exceeding 3 bars of pressure). After this initial concentration step, the retentate was subjected to diafiltration (6 diafiltration volumes; using 1×DPBS). After diafiltration, the retentate was further concentrated, to produce a total volume of at least 100 mL. The parameters of the TFF process were as follows: feed manifold pressure (PT01)—0.94-2.1 bars; retentate manifold pressure (PT02)—0.12-0.13 bars; retentate manifold flow rate (FT01)—0.012-0.58 L/min; transmembrane pressure (TMP01)—0.53-1.11 bars; and quattroflow pump (P01)—14-20%.

### Example 13

### Formulation/Composition

After enrichment and concentration by TFF, the final sEV formulation was then prepared by filter sterilizing the resulting retentate using a 0.22 μm filter (Sterivex™-GP Pressure Filter Unit, 0.22 μm, Millipore, Ref: SVGPL10RC). In some experiments, 25 mM trehalose was added before this sterilization step to avoid aggregation. After the sterilization step, the final formulation (with or without the addition of 25 mM trehalose) was bottled into glass vials (2 mL, bromobutyl cap; Adelphi Ref: VCDIN2RDLS1). Final product formulation was then stored at −80° C. for future use or testing. Additionally, final formulations were also tested in which the retentate was first frozen and stored at −80° C. before sterilizing filtration using either a 0.22 μm filter (Sterivex™-GP Pressure Filter Unit, 0.22 μm, Millipore, Ref: SVGPL10RC), or a Sartopure 2 filter (Pore sizes (prefilter+filter): 0.45 μm+0.2 μm; Sartorius Ref: 5441307H4-OO-B) to produce final formulations thereof, as shown in FIG. 24B.

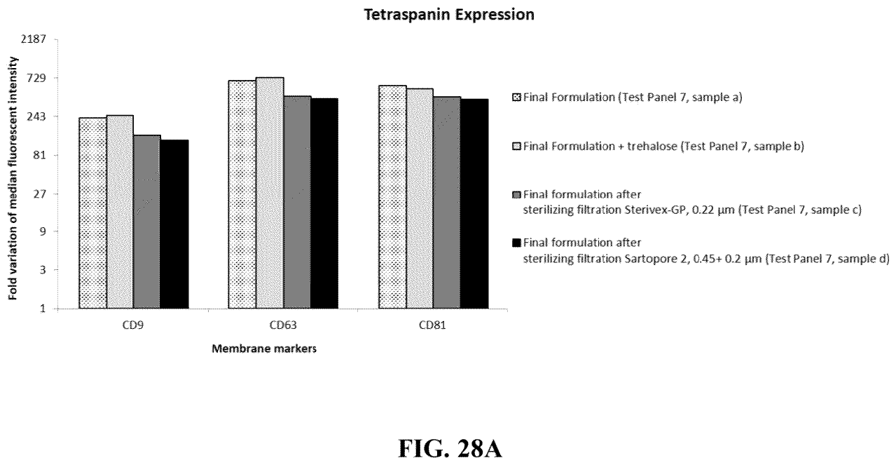

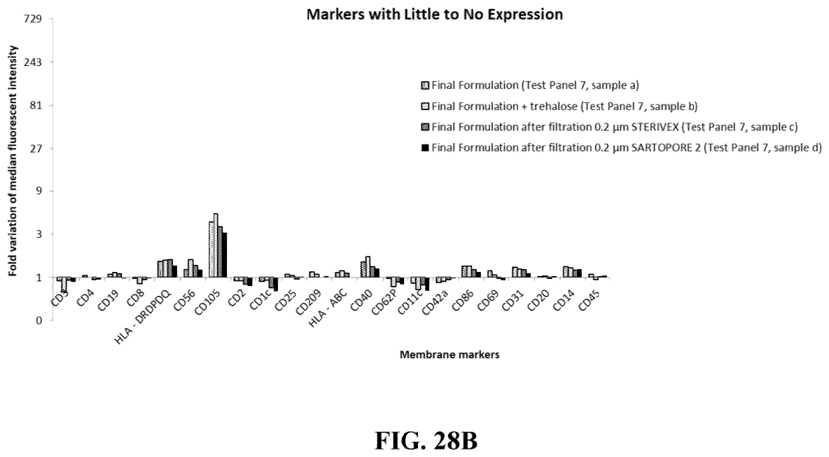

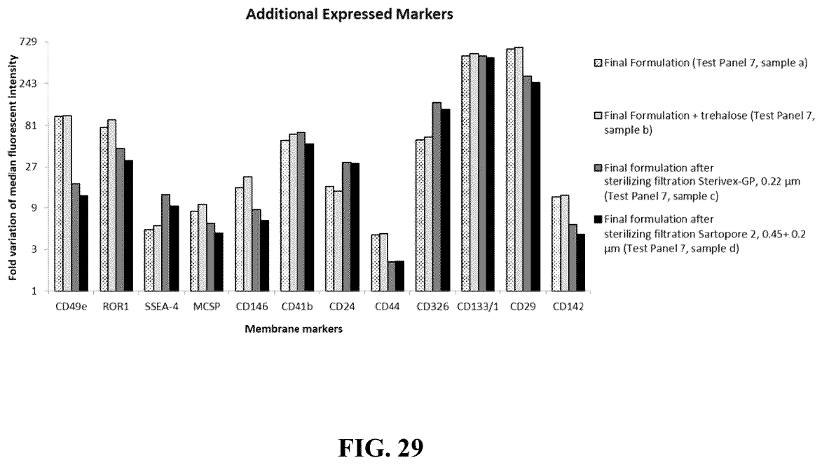

The final formulations, therefore, were in PBS (with or without trehalose), and were positive for CD9, CD63 and CD81 (canonical EV markers), as well as positive for the cardiac-related markers CD49e, ROR1, SSEA-4, MSCP, CD146, CD41b, CD24, CD44, CD236, CD133/1, CD29 and CD142, as detected by MACSPlex (as shown in FIGS. 28A and 29).

### Example 14

### Characterization of the Identity of CPCs During Vesiculation in the GMP-Compatible Process

To assess the identity of the cells during the vesiculation process in Example 12, the D+0 CPCs, as well as the harvested cells at D+3 and D+5, were analyzed by flow cytometry. iPSCs and cardiomyocyte (CM) cells were included as controls. As shown in FIG. 25, flow cytometry analysis, performed using a MACSQuant 10 Flow Cytometer with iPSC-, CPC- and cardiac-markers, demonstrated that the CPCs became more mature over the five-day vesiculation period. Specifically, the CPCs maintained little to no Nanog or SOX2 protein expression, and exhibited a continued increase in CD56, cTNT, and aMHC, protein expression (however, they did not reach expression levels of CD56, cTNT, and aMHC similar to cardiomyocytes, indicating that they remained progenitors throughout the process). iPSC and CM control cells were analyzed separately, and the average values are presented in FIG. 25 for comparative purposes.

### Example 15

### Analysis of EV Particle Concentration and EV Particle Size Distribution in the GMP-Compatible Process

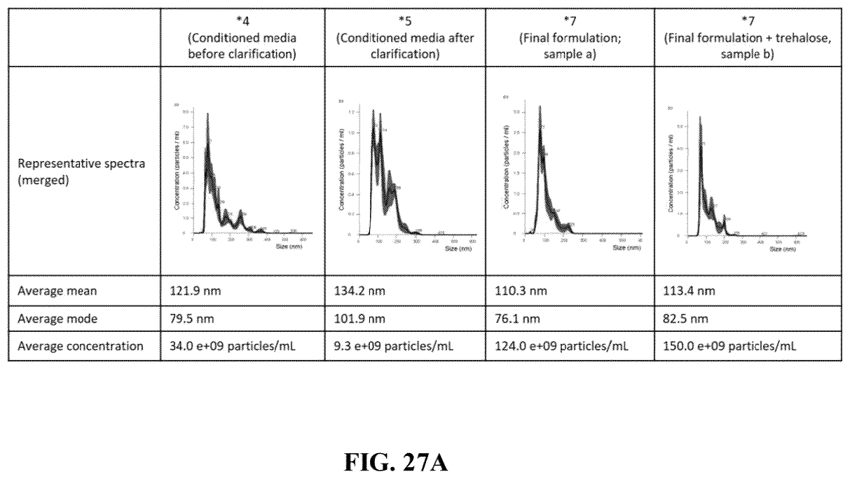

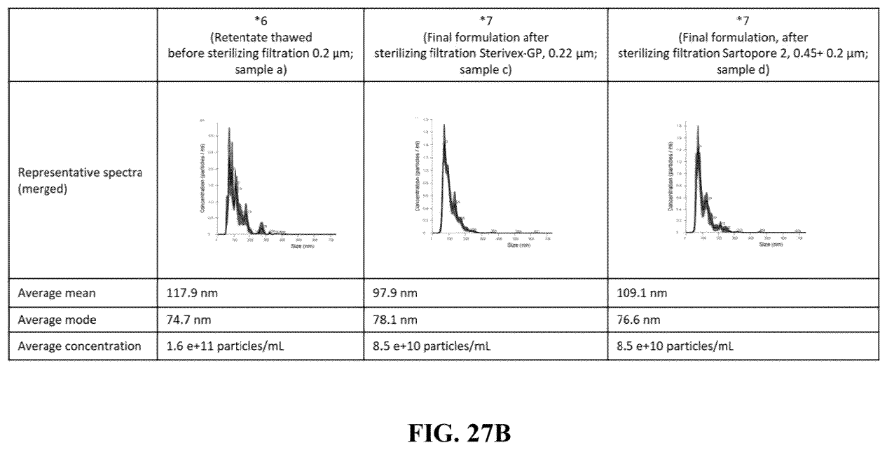

To assess the particle concentration and size distribution of EVs produced in Example 12, conditioned media prior to clarification (*4) and after clarification (*5), and the final formulations (with and without trehalose, samples b and a, respectively), were analyzed by nanoparticle tracking analysis (NTA; NanoSight). FIG. 27A depicts representative size distribution curves for each sample. The overall size distributions, means and modes, were similar between samples. A peak was observed generally between 50-150 nm, corresponding to the size of exosomes or small microparticles. The TFF step resulted in an approximately 32-fold concentration of particles. Similar experiments were also conducted on the previously-frozen retentate and final formulation samples (filtered with STerivex-GP or Sartopore 2) depicted in FIG. 24B (“*6,” sample a; and *7, samples c and d). The results of these experiments are shown in FIG. 27B. The TFF step resulted in an approximately 20-fold concentration of particles, even though particles were lost during final sterilizing filtration (especially for the final formulations produced from thawed retentate).

### Example 16

### Analysis of EVs Produced by the GMP-Compatible Process for EV Markers

To assess the presence of EV markers in the clarified conditioned media (before TFF) and the final formulations (with and without trehalose) in Example 12, a MACSPlex Exosome Kit human (Miltenyi Ref: 130-108-813) was used to identify and quantify the presence of EV markers. As shown in FIG. 28A, the analysis confirmed the presence of extracellular vesicle tetraspanins (CD9, CD81 and CD63) in both the conditioned media (before TFF), and in the final formulation (with and without trehalose). Further still, as shown in FIG. 28B, the MACSPlex analysis also revealed a variety of markers that were found to be present either in low amounts (e.g., CD3, CD4, CD8, HLA-DRDPDQ, CD56, CD105, CD2, CD1c, CD25, CD40, CD11c, CD86, CD31 and CD20); or were substantially absent (CD19, CD209, HLA-ABC, CD62P, CD42a and CD69), in the conditioned media (before TFF), and/or in the final formulation (with and without trehalose).

Additionally, as shown by FIG. 29, additional cardiac-related markers were also observed in the conditioned media (before TFF), and in the final formulation (with and without trehalose).

### Example 17

### In Vitro Analysis of the Potency of EVs Produced by the GMP-Compatible Process

To analyze the functionality and potency of the final formulations produced by the GMP-compatible process in Example 12, two in vitro assays were used: a HUVEC scratch wound healing assay; and a cardiomyocyte viability assay using staurosporine-treated human cardiomyocytes.

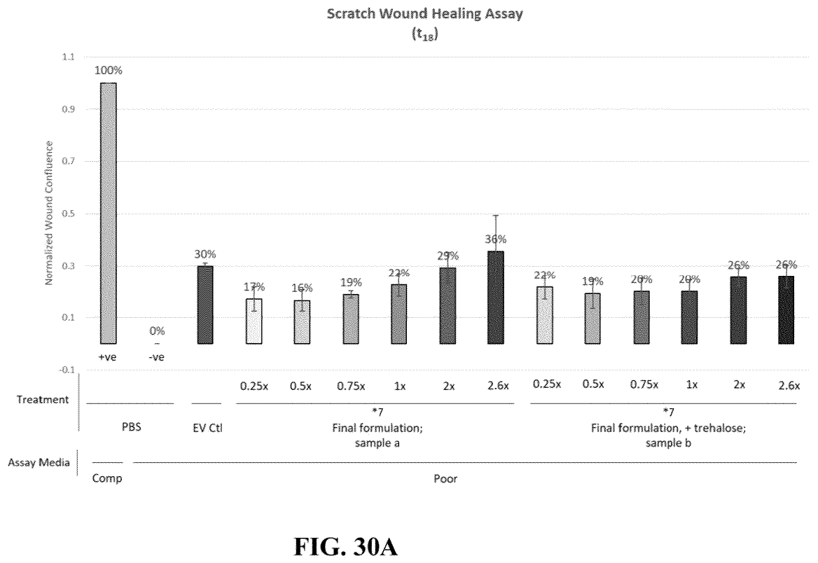

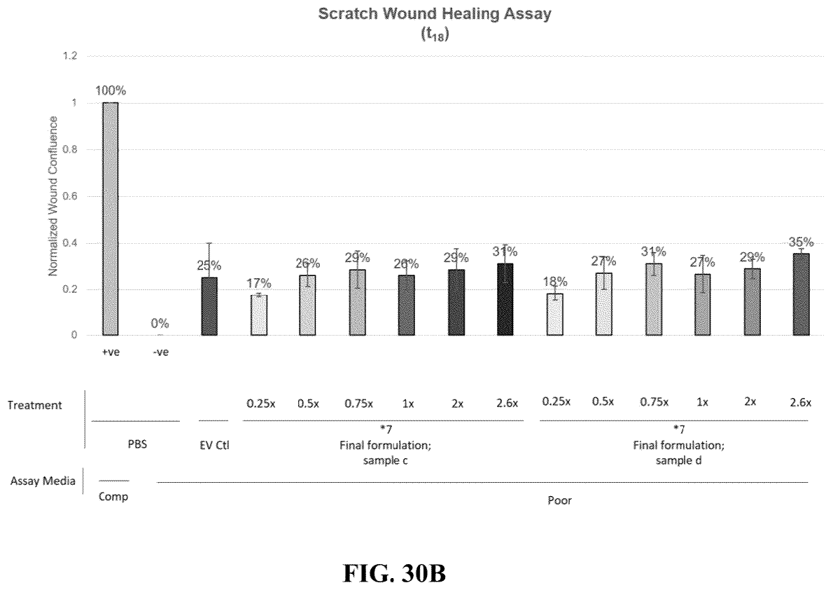

For the HUVEC scratch wound healing assay, a scratch wound healing assay (developed by Essen BioSciences, for the Incucyte) was employed, according to the manufacturer's directions. Briefly, HUVEC cells were expanded using HUVEC Complete Media: Endothelial Cell Basal Media (PromoCell, Ref: C-22210), supplemented with the Endothelial Cell Growth Medium Supplement Pack (PromoCell, Ref: C-39210). After expansion, the cells were cryopreserved in CS10 (Cryostore, ref: 210102) at 1-2×10⁶ cells per aliquot (enough for between a half to a full 96-well plate). Two days prior to assay, HUVEC aliquots were thawed, and plated onto ImageLock 96-well plates (EssenBio, Ref: 4379) at 10,000 cells/well, and grown in HUVEC Complete media for two days. Cultures were maintained at 37° C. (atmospheric oxygen, 5% CO₂) throughout the maintenance and assay process. Wells were scratched using a Wound Maker (EssenBio, Ref: 4493) according to the manufacturer's directions, and cells were then rinsed with Endothelial Cell Basal Media and cultured overnight (either in HUVEC Complete Media with PBS, as a positive control; in Endothelial Cell Basal Media with PBS, as a negative control; or in Endothelial Cell Basal Media supplemented with sEV preparations in PBS). Using an Incucyte with the Scratch Wound Healing Module, plates were imaged at 18 hours after treatment. Wound closure was determined using the manufacturer's software, and values were baseline (negative control) subtracted, and normalized to the positive control. FIG. 30A depicts that the final formulations with and without trehalose (*7, samples b and a, respectively) promoted wound healing. FIG. 30B depicts that the previously-frozen final formulations without trehalose (*7, samples c and d) promoted wound healing.

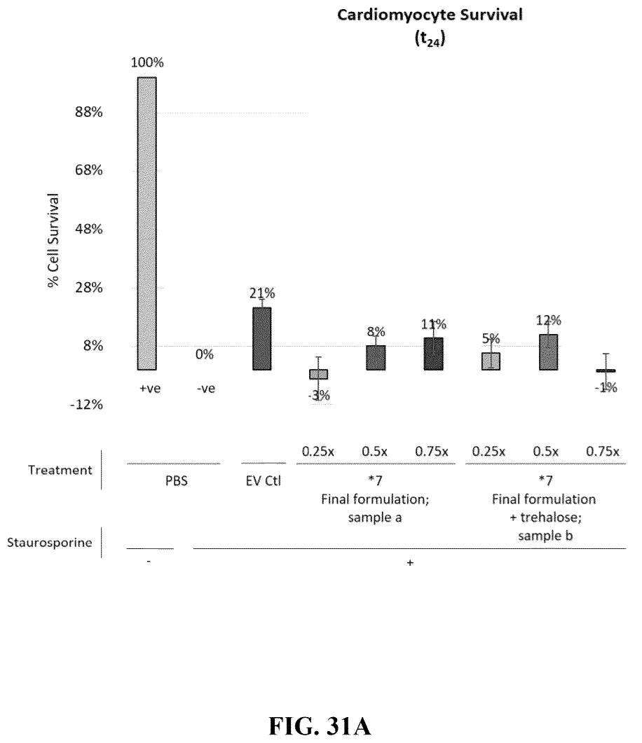

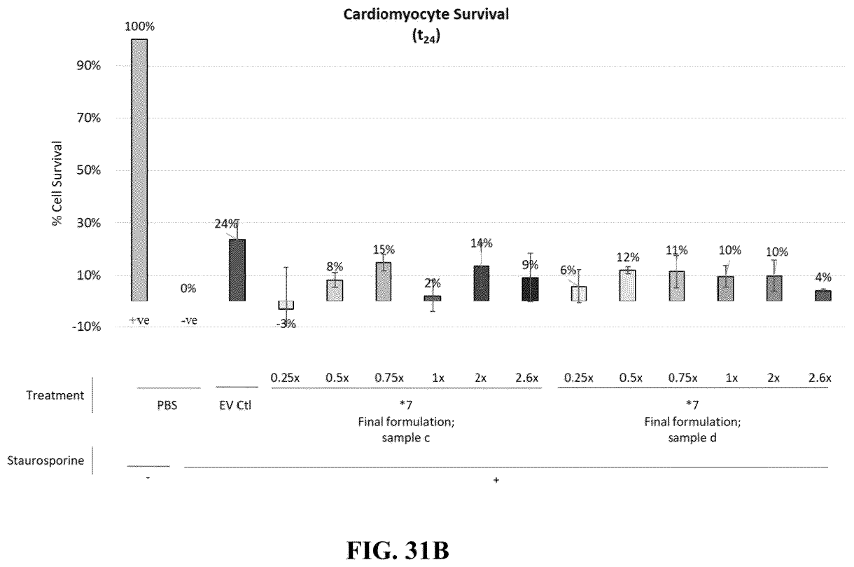

For the cardiomyocyte viability assay using staurosporine-treated human cardiomyocytes, iCell Cardiomyocytes2 (Fujifilm Cellular Dynamics, Inc., ref: CMC-100-012-001) were plated at 50,000 cells/well of a fibronectin-coated 96-well plate in iCell Cardiomyocyte Plating Medium (Fujifilm Cellular Dynamics, Inc., ref: M1001), and cultured for 4 hours. The media was then exchanged for iCell Cardiomyocyte Maintenance Medium (iCMM, Fujifilm Cellular Dynamics, Inc., ref: M1003), and cells were cultured for up to 7 days, with full media exchanges every 2-3 days. After a minimum of 4 days, cells were exposed to iCMM with NucSpot Live 650 dye (Biotium, ref: 40082) (this served as a viable cell control); or to iCMM with NucSpot Live 650 dye, and staurosporine (Abcam, ref: ab146588) at a final in-well concentration of 2 μM (this also served as an apoptotic cell control). Dye, PBS, and DMSO concentrations, and final well volumes, were equivalent in all wells. Cells were cultured in these pre-incubation media for four hours. After this incubation, the pre-incubation media was removed, and the wells were rinsed with iCMM. Cells were then fed with iCMM with NucSpot Live 650 dye and PBS, or iCMM with NucSpot Live 650 dye supplemented with increasing concentrations of sEV preparations while maintaining PBS final volumes. Wells were imaged in an Incucyte at 24 hours, and nuclei counts were determined. FIG. 31A depicts that the final formulations with and without trehalose (*7, samples b and a, respectively) promoted cardiomyocyte survival. FIG. 31B depicts that the previously-frozen final formulations without trehalose (*7, samples c and d) promoted cardiomyocyte survival.

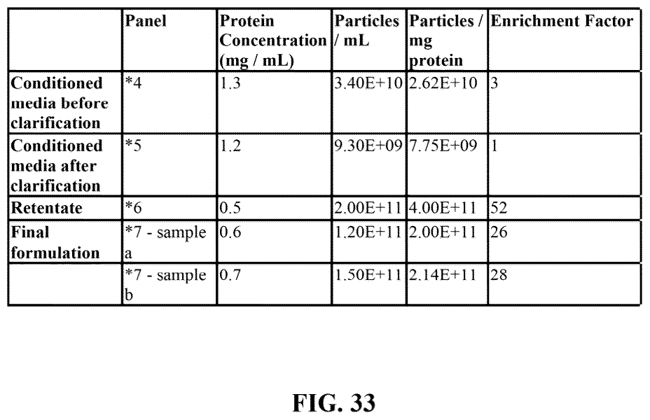

The testing panel used with respect to the processes/products of Example 12, and as embodied, e.g., in Examples 13-17, is shown in FIG. 21. Results therefore are shown in FIG. 32. Additionally, FIG. 33 depicts the degree of enrichment (as calculated by the increase of particles per unit protein), as compared to conditioned media after clarification, for the retentates and final formulations produced in Example 12.

### Example 18

### Analysis of the Effect of Cardiovascular Progenitor Cell (CPC) EVs on Cardiac Function in a Mouse Heart Failure Model

To analyze the in vivo functionality and potency of sEV preparations produced in accordance with methods described in the present disclosure, a mouse model was used to determine the effect of sEV preparations on cardiac function (in mice in which heart failure had been induced).

Heart failure was induced in C57BL/6 mice essentially as described in Kervadec et al. (J. Heart Lung Transplant, 2016, 35(6): 795-807; incorporated by reference herein in its entirety). Briefly, surgical occlusion of the left coronary artery was performed in 42 mice in total, to induce chronic heart failure (CHF). At three weeks post-occlusion, 22 of the mice were treated with either PBS vehicle control (60 μL, n=11) or sEV (60 μL, n=11), delivered by percutaneous injections under echocardiographic guidance into the peri-infarct myocardium (as described in Kervadec et al.). The administered sEV was produced in accordance with the “sEV 5.3” scheme depicted in FIG. 2 (whereby the sEV was prepared by ultracentrifugation from clarified “MC5”), and the resulting EV were resuspended in half the typical PBS volume (to generate a 2-fold concentrated sEV preparation, containing the secretome from 6.22E+04 cells per μL of sEV preparation).

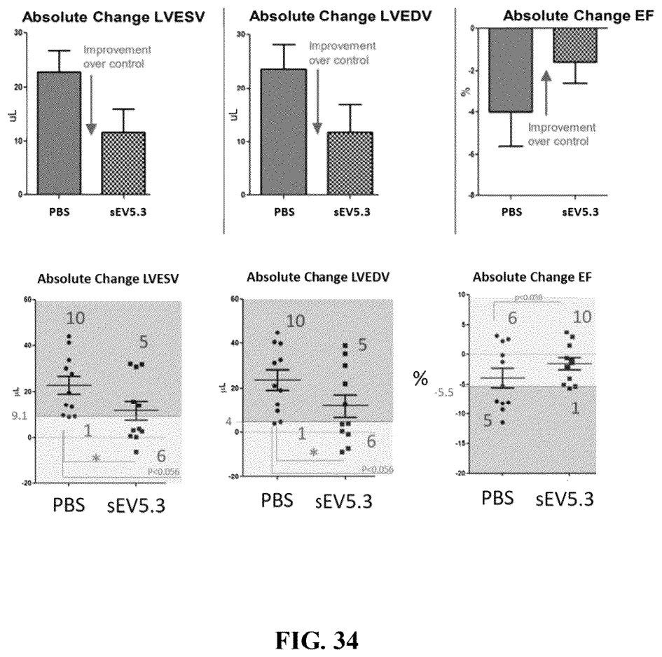

At four weeks post-occlusion, cardiac function was assessed by echocardiography. The results thereof are shown in FIG. 34. Amongst the CHF mice, significantly fewer sEV-treated mice (as compared to the PBS-treated mice) had severely progressive heart failure (defined here as a greater than 14% increase in Left Ventricular End Systolic Volume, LVESV; p<0.05). Further, although not statistically significant, the Average Ejection Fraction of the PBS group deteriorated 2.5-fold more than the sEV-treated group (−4% vs −1.6%, respectively; ns). The results confirmed the ability of the sEV preparation to improve cardiac function in vivo.

1. A method for generating a secretome, said method comprising:
(a) culturing one or more progenitor cells in a first serum-free culture medium, wherein said first serum-free culture medium comprises basal medium, human serum albumin, and one or more growth factors;
(b) removing said first serum-free culture medium from said one or more progenitor cells;
(c) culturing said one or more progenitor cells in a second serum-free culture medium, wherein said second serum-free culture medium comprises basal medium, but does not comprise human serum albumin or growth factors; and
(d) recovering the second serum-free culture medium after the culturing of step (c), to thereby obtain conditioned medium comprising the secretome of the one or more progenitor cells.

2. The method of claim 1, wherein said method further comprises concentrating, and/or enriching for, a small extracellular vesicle-enriched fraction (sEV) from the medium recovered in step (d).

3. A secretome-containing composition obtained by the method of claim 1.

4. An sEV-containing composition obtained by the method of claim 2.

5. A method for producing a therapeutic composition suitable for administration to a patient, said method comprising producing a secretome-containing composition according to the method of claim 1.

6. A method for producing a therapeutic composition suitable for administration to a patient, said method comprising producing an sEV-containing composition according to the method of claim 2.

7. A therapeutic composition, wherein said therapeutic composition comprises the secretome-containing composition of claim 3, and a pharmaceutically acceptable excipient or carrier.

8. A therapeutic composition, wherein said therapeutic composition comprises the sEV-containing composition of claim 4, and a pharmaceutically acceptable excipient or carrier.

9. A secretome-containing composition obtained by the method of claim 1, wherein said one or more progenitor cells comprise progenitor cells selected from the group consisting of cardiomyocyte progenitor cells, cardiac progenitor cells, vascular progenitor cells and cardiovascular progenitor cells.

10. An sEV-containing composition obtained by the method of claim 2, wherein said one or more progenitor cells comprise progenitor cells selected from the group consisting of cardiomyocyte progenitor cells, cardiac progenitor cells, vascular progenitor cells, and cardiovascular progenitor cells.

11. A therapeutic composition, wherein said therapeutic composition comprises the composition of claim 9, and a pharmaceutically acceptable excipient or carrier.

12. A therapeutic composition, wherein said therapeutic composition comprises the composition of claim 10, and a pharmaceutically acceptable excipient or carrier.

13. A method for treating acute myocardial infarction or heart failure, comprising administering to a subject in need thereof the therapeutic composition of claim 11.

14. A method for treating acute myocardial infarction or heart failure, comprising administering to a subject in need thereof the therapeutic composition of claim 12.

15. A method for improving angiogenesis, comprising administering to a subject in need thereof the therapeutic composition of claim 11.

16. A method for improving angiogenesis, comprising administering to a subject in need thereof the therapeutic composition of claim 12.

17. A method for improving cardiac performance, comprising administering to a subject in need thereof the therapeutic composition of claim 11.

18. A method for improving cardiac performance, comprising administering to a subject in need thereof the therapeutic composition of claim 12.
